SEQUENCE LISTING (41 sequences)

SEQ ID NO: 1 - DNA, 9367 bp, Human
  mRNA (1)..(87): Exon 1
caggtgcagcggccatggctacagcaagacccccctggatgtgggtgctctgtgctctgatcacagccttgcttctgggggtcacaggtaaccagaactctggggtgggagggttgtgggattgggaggactgtctctgcggcactagagcgcctgtcccctggggaactgtgtgagcctgggcatgactccgggaccgggtgaatgtgagtctctgtctgtacttgtggttgtgcgatcgtatgtggccctgtgactgccacggtgtgtgtcggggagggggatgccttttcccatatcaggtgactgtgcggcaggtggcactgaccctttgaggctgtgtgtgtggttttgtgattgtgtgtgcatttaagattgtgtgtggctccacagctgtgtgggtgaatgcatgtagcactgggggtgttcactgtgtgtttggctgtgtgtggtgacttggcattgtatatgactgcaggtatctgcagttcctgtccctgaggtcccgggattgcgtgcaacaaaagtggtcatcaccatggaaagctgtgactgtgtgctgcttgcaggcgattatgtgattgtggctgagtgtgacgttatggatgcccgtatttgtgaccgtgtgactacctgaagctctgtgtaggggtgactgtatgtgactgtgtgtgtctgtgtgaggccgtgtaaatgctactgtatgtgtgatggtgcagctgtgtgtctggagtttctgtctctgcctggagggatagagggtgcaggggtagctatctctgggagatgggtgccaggtgactgacttgcagtgtgtgcctgtgtgcagaagagtatgtggcagtctgaacatctgtgcacacacggcatctgtgcgtggcactgagacactgtggatgagggtgtgcgatcccgctaggctgcccgggagcgtgtgtacctggagacagagctgtatgttagctgcacctgtggaggcaacatgggcgtgtctgcagaactgcgtgcgtgcttggctgttactgctgttgtgcgcgtggttcttggggtgagttcgtgaatgatggtggtgccagggccatcagcaagggtaagaaccaggccgggcgcggtggctcacgcctgtaatcccagccctttgggaggccgaggcaggcggatcacctgaggtcgggagatcgaggccagcctgaccaacatggagaaccccgtctctactaaaaatacaaaaaattagctggtgtggtggcgcgtgcctgtaatcccagctactcgggagactggggcagaaaaatcgcttgaacccgggaggtggaggttgcggtgagccgagatcgcgccattgcactccagcctgggcaacaagagcgaaactccgtctcgaaagaaaaaaagaaaaaaaaaagggtaagaaccagtgaatgggcacgggaggactgatgatggagtggggcatgcatgtagtctgtaggtctgtgtgtgagaggaggagattgacaggattgagaaggcatgttttcatctgagaattcagaaacctaggcctgctcttcccctccatgtggccccctaagctgagcccttctttcctggtcctgctttcggaaccctagctccgcccatgagctctgaccccacctcctttcctcaaccacgcccctaggccagactctagtggaccccgcctaaggccacacccctttgggccaggctccaccccctattctgtgggtaccttctagaacccccttcaaagtcagagcttttttttttttttttttggagacagtcttgctctctctcccaggctggagtgcagtggcgtgatctcggctcactgcaacctctgcctcccaggttcaagtgattctcgtgcctccacctcctgagtagctgggattacaggtgcgcgccaccacgcctggctaatttttgtgtctttagtagagacagggtttcaccttgttggccaggctggtctcaaactcccaacctcaggtgatccgcccacctcggcctcccagagtgctggggttacaggcgtgagccaccgcccccagcccaaagtcagagctctttataggagactctaacatgtaaccctgaccctggccctaactaagtcaattccaaaccccttcctgcctccagccctgaccccactcactgaggcctgaccccacttcttgagaccagttccatccctaaagccctggtctccctcccatccccaggctccagcccccacagctttggcactacccctgagcttgtccaggaatcctgtacccaattttaccctcacatgtagttctagccaattccaggaatctgtgaggtccagttagagtccagtaaccctacctgagcctgggctctgtccttgagcttgagcctgggcttgagaggtgccactcttattctccaggccctgcccctgccccctcagcatgtcagacacccaccctctagctggtctggcctcttgagtctgaaacccacccccagcccaagccccgcctctgagccccgcccaacccattttccgttcccagagcatgttctcgccaacaatgatgtttcctgtgaccacccctctaacaccgtgccctctgggagcaaccaggacctgggagctggggccggggaagacgcccggtcggatgacagcagcagccgcatcatcaatggatccgactgcgatatgcacacccagccgtggcaggccgcgctgttgctaaggcccaaccagctctactgcggggcggtgttggtgcatccacagtggctgctcacggccgcccactgcaggaagaagtgagtgggagttccaagaggagggttggtggggacggggaagtgggggtgggggtggggaagtgggggtgggggtgtcatggaggtgagggctggtggggacggggaagtggggttgggggtgtcatggaaggtgagggttggtggggatgggttggggatgtgggagcaggaggaggtcgagttggggataggactaaggatggagttttgcgggggagcaaggtgggaggatgaggttggagaggggagagtgttgtggtagggaatgggaaggagccaaggatgggttggatttggggttaggagcatatatttgttgaatggtttgggatggaggtggaattgggattggctttagaattgggggtgggtgaaaatcgggctggggtggaaatgaagatagcatggagatagggttgagattgggagcagatatagaatgaaggatggggattggagttttgggtggggttggagatggttggatttgggcttgagaatgcatatggtgatggcttctgggtagggaaagaattagggttgggaatgggatgggtttggaattgtgactgggatggggacaggcatgggattggagaccaagagggagttgaggatggtttggggaccgggggtggggatgggggtggggctggggctgggtgtggggttgggattggcgttggacgtggagatagagatcagggttggtggtgacctgccccatcttcctcagagttttcagagtccgtctcggccactactccctgtcaccagtttatgaatctgggcagcagatgttccagggggtcaaatccatcccccaccctggctactcccaccctggccactctaacgacctcatgctcatcaaactgaacagaagaattcgtcccactaaagatgtcagacccatcaacgtctcctctcattgtccctctgctgggacaaagtgcttggtgtctggctgggggacaaccaagagcccccaaggtgagtgtccaggttcttcttgataccgacccatctctgccgccttccatctttctccacttctcattgtgttcctgtttgacagtgcacttccctaaggtcctccagtgcttgaatatcagcgtgctaagtcagaaaaggtgcgaggatgcttacccgagacagatagatgacaccatgttctgcgccggtgacaaagcaggtagagactcctgccaggtgaggacacctctctttattcagcagatacacactgagtgccaactcggtaacatggagcgttgccaaattctgagaatccagcaattgccaagacagtcaggacccctgttctcacagagctcataccctagagtagtggtgtttagtagaaataatgctgagctgcttatgtcatttccagttttttagtagccacattaaaacaggtaaaaaaggctgggcgcagtggctcacacctgtaatcccagcactttgggaggctgaggcaggcagatcacctttggtcaggagtttgagactagcctggccaacatggcgaaactctgtctctaaaaaaaaatacaaaaattagcctggcatggtggcgggcgcctgtaatctcagctgctcaggaggccgagacacaagaatcacttaaacccaggaggtggaggttgcagtgagctgagatcgtgccactcactccaacctgggagacagagtgacacttttgtctcaaaaagaaaaaaaaaaacaagtaaaaaagaaacaggtgaagttaactttaataacccaatgtatcccaaatacaatcatttcaaagtgtaattaatataaaacaattatgaatgagatactttacattcttttcttgttttcatattaagtctttgaaagtgagtatatatgttatgctgacagcacatctcaatttggactagctacatttcaggtgctcagtagccacatgtggctagcagttactgtattggatggcacggatctagagggaaagatcagggctgttttgtatggttgggcaggttgtgcactgcataaagataccatatctaataggggcactccgtgttacagatgtcagttttggcagttttcaggcgtgtggtagttaagtgtcttgtttcaacaaaatctgtaatatgacagttttctagcaagtgctggtaaaatatcttgaggaaggaaaagagaaatctggtaggtatttttacaagagaatatttaatacaggggattaattgcaaagctgctggaagggctggaggaacaaagttaaaaaataaaaaactctgtggtcaagaatctgcataaatagggcaatttcagagagtggtaaaggttaaccccaaaataaaacatggttttaggatagtaaacaataagggccaatattcaaaaaggtggtcaggggagcctccttggagaggtggcatttgagcagagaatggatgacacaaagaagctaaactcgtgaagtttaaggggaaagaaaaggcacgtgcaaaggccctgaggcagtaaggaatttggctgattcaaagaagaagaggaaaccaatgcaactggagaacaaaagtgggggcaacagtagaaagtgacgctggaggtgtaggcaggggcgaatgctctgcaagtatttcttggtcaccaacacagagcttccctatgttctaatggaagctgtatctgttgaggaagacagaatttaaaatcaaactgttacatcaaccagcacccttctctgtattcaggctcccaagggatctagaaggacgtaagttaacaagctctcattagcagggtgtgtgtttcaacagtagttaggaagctggggattcaggagtactccagtcccatggctatgaaaagctccccccaaattgtacaaacctgacaaatgcaacacctccccagctctccccatttcttctctgtgccctgggtgtgggggggtgggttgcgagggggaaaacttttaacagaagaaagcacatctcggccgggcgtggtggctcacacctgtaatcccaacactttgggaggccgaggcgggtggatcactaggtcaggagatggagaccatcctggctgacacggtgaaaccctgtctctactaaaaacacaaaaaattagccgggcgtggtggcaggcgcctgtagtcccagctactcgggaggctgaggcaggagaatggcctgaacccgggaggcggaacttgcagtgagccgaggttgcaccactgcactccagcctgggcaacacagtgagactccgtctcaaaaaaaaaaaaagaaaagaaaagaaatcacatctcattcaagtggtggcatttaaaactatttagcctttctgtaggcaaggttagtatcttgtttttccagacctcaaggtgtttttttgtttgttttttcataccggtgtgtggtctgggtgtggccactaaaagctacaagcaagaaataataacaactacaacaatactaataccaatagtataaaaataatagcatctggctaattgctggacactgttttaagtggtttgcatgcctcagctcattaactcatttacctgttattattggccctattttacaaacaaggagccaaggctcagagcagttaactaacagcctctcaaaagaaactctgcagagatattaaatttaaaaaataatgagagaaattaaaccacaagaaagttgaaatttagaggtacaggcagctaagcttgtttgctttgaaacagtgtctgctactgggaaaaaggcaagtcttggctttcctaataattgataccaggactctgtaattcatattttgcatgcatgtaagtaagaaatgaagccgggtgcaatggcacatgccagtaatcccagcactctgggagactgaagtgggaagatcacttgagctcaggagttcaagaccagcctgggcaactaaaaattaaaaaaataaaaatactaattgtttttattttagtagattttattcataccacttacatcattattgtagtatgtacatatttatttcttttcttttcttttcttttcttttttgagacggagtctcgctctgtcacccaggctggagtgcaatggcaccatatcagctcactgcagcatgcgcctcctgggttcaagcatttcttccacctcagcctcccaagtagctgggataacaggcacccaccaccatgcctggctatttttttttttccgtagagatggggttccaccatgttggccaggctggtcttgaactcctgacctccagtgatctgcctgcctcggcctcccaaattgctggtattacaggtgtgagccaccgtgcccaggtgggagatagacatttctctctacctcaaacagaggtccactcaagctacttttcattttcttcataaatattagccgagtggctattttgcaccaggaatggttccaggtgctgtggatatggcatcaggcaaaacagaccaaaaacttcctgccgcgtggacctcatgttccccaagtggaagacaggcaataaagagatagataaatatgtagtaaattaaaaaaaaaaaaaattagccgggtgtggtggcttgcacctgtagttccagctacttgggaggctgaggtgggagaattgcttgagcccaaacgtttgaggctgcggtaagccatgactgcactgctgcactccagacagcagcctgggtgacaaagcaagacgtttttgtcagaaagaaaaaaaaaagagacgaagggaggaaggagagagaaaggaaggaaggaaggagaaagaaaggaaggaaggagaaagaaaggaaggaaggaaggagaaagaaaggaagaaagagaaagaaagaaaaagaaagaaagaaagaagaaagaaaagagagaggaaggaaggaaagaaggaaaagagggaaaaaaatgactgttgaagagcagtgagtattattataggagggtaattatagggaggtatggggaattgaagacaggaaacacaaattagtccaagcgaatggatttctattgggagtgattctgcccctagaagacactggcaataccaggagacatttttggttgtcacaactatatggaggggcattactggcaactaatggatagatgccaagtgtgctgttcaacatgctatgatgcacacggcaggcctccacaacaaaccattatccagcttcagatgcccacagtgcccagatcgaggaaccctcatccaggggctgagaaccgtatttttgcagaagggaggtataaggatgggttggtggagaatggggaaggaaggtgtgtgtccagtaagagaaataaggcctgcacaggctggaggggagagtgagagagaaagggaggcggagagatacacgatgagggagacaggctggaacagaaagtagagacgaagattcgagatgtggagaggaagggtcacagacccccccgaaatgatgtgtggacaacaggaatctggaagaggaagatggagtggagagtgacaaatggggtctaaaggttgaacttggaggccaggcatggtggctcacgcctgtaatcccaacactttggaggctgaggtgggcgaatcacttgaggccaggagttcgagaccagcctggccaacatggtgaaaccccgtctctacaaaaaaaatacaaaaaattagccgggtgtggtgatggacacctgtagtcacagctacttgggaggctgaggcaggagaattgcttgaacccgggagatggaggctgcagtgagctgaggtcaggccactgcgctccaacctgggcaacagagtaagactccatctcaaaaaaaaaaaagctggatttggagtgaaatattaataacattctccctctctctccttttgcctgtgtctccatctctgtctttttctgcatttcttcatctctgtactttccatctctgtgtgtctgttcccatctgcttctccatctatgggcatctctgggtctctcatgtctccttctgcccactttgccacatctctgcctctctcatgcccccctttctctcctgcagggtgattctggggggcctgtggtctgcaatggctccctgcagggactcgtgtcctggggagattacccttgtgcccggcccaacagaccgggtgtctacacgaacctctgcaagttcaccaagtggatccaggaaaccatccaggccaactcctgagtcatcccaggactcagcacaccggcatccccacctgctgcagggacagccctgacactcctttcagaccctcattccttcccagagatgttgagaatgttcatctctccagcccctgaccccatgtctcctggactcagggtctgcttcccccacattgggctgaccgtgtctctctagttgaaccctgggaacaatttccaaaactgtccagggcgggggttgcgtctcaatctccctggggcactttcatcctcaagctcagggcccatcccttctctgcagctctgacccaaatttagtcccagaaataaactgagaagtgg

SEQ ID NO: 2 - protein, 293 aa, Human
MATARPPWMWVLCALITALLLGVTEHVLANNDVSCDHPSNTVPSGSNQDLGAGAGEDARSDDSSSRIINGSDCDMHTQPWQAALLLRPNQLYCGAVLVHPQWLLTAAHCRKKVFRVRLGHYSLSPVYESGQQMFQGVKSIPHPGYSHPGHSNDLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 3 - DNA, 9755 bp, Human
  mRNA (1)..(314): Exon 1
tcaggcccggtgcccagagcccaggaggaggcagtggccaggaaggcacaggcctgagaagtctgcggctgagctgggagcaaatcccccaccccctacctgggggacagggcaagtgagacctggtgagggtggctcagcaggaaggaaggagaggtgtctgtgcgtcctgcacccacatctttctctgtcccctccttgccctgtctggaggctgctagactcctatcttctgaattctatagtgcctgggtctcagcgcagtgccgatggtggcccgtccttgtggttcctctctacttggggaaatcaggtaggggagggaggggaagtgggttaagggctccccggatcgcctgggcctcccaaccctctgacattccccatccaggtgcagcggccatggctacagcaagacccccctggatgtgggtgctctgtgctctgatcacagccttgcttctgggggtcacaggtaaccagaactctggggtgggagggttgtgggattgggaggactgtctctgcggcactagagcgcctgtcccctggggaactgtgtgagcctgggcatgactccgggaccgggtgaatgtgagtctctgtctgtacttgtggttgtgcgatcgtatgtggccctgtgactgccacggtgtgtgtcggggagggggatgccttttcccatatcaggtgactgtgcggcaggtggcactgaccctttgaggctgtgtgtgtggttttgtgattgtgtgtgcatttaagattgtgtgtggctccacagctgtgtgggtgaatgcatgtagcactgggggtgttcactgtgtgtttggctgtgtgtggtgacttggcattgtatatgactgcaggtatctgcagttcctgtccctgaggtcccgggattgcgtgcaacaaaagtggtcatcaccatggaaagctgtgactgtgtgctgcttgcaggcgattatgtgattgtggctgagtgtgacgttatggatgcccgtatttgtgaccgtgtgactacctgaagctctgtgtaggggtgactgtatgtgactgtgtgtgtctgtgtgaggccgtgtaaatgctactgtatgtgtgatggtgcagctgtgtgtctggagtttctgtctctgcctggagggatagagggtgcaggggtagctatctctgggagatgggtgccaggtgactgacttgcagtgtgtgcctgtgtgcagaagagtatgtggcagtctgaacatctgtgcacacacggcatctgtgcgtggcactgagacactgtggatgagggtgtgcgatcccgctaggctgcccgggagcgtgtgtacctggagacagagctgtatgttagctgcacctgtggaggcaacatgggcgtgtctgcagaactgcgtgcgtgcttggctgttactgctgttgtgcgcgtggttcttggggtgagttcgtgaatgatggtggtgccagggccatcagcaagggtaagaaccaggccgggcgcggtggctcacgcctgtaatcccagccctttgggaggccgaggcaggcggatcacctgaggtcgggagatcgaggccagcctgaccaacatggagaaccccgtctctactaaaaatacaaaaaattagctggtgtggtggcgcgtgcctgtaatcccagctactcgggagactggggcagaaaaatcgcttgaacccgggaggtggaggttgcggtgagccgagatcgcgccattgcactccagcctgggcaacaagagcgaaactccgtctcgaaagaaaaaaagaaaaaaaaaagggtaagaaccagtgaatgggcacgggaggactgatgatggagtggggcatgcatgtagtctgtaggtctgtgtgtgagaggaggagattgacaggattgagaaggcatgttttcatctgagaattcagaaacctaggcctgctcttcccctccatgtggccccctaagctgagcccttctttcctggtcctgctttcggaaccctagctccgcccatgagctctgaccccacctcctttcctcaaccacgcccctaggccagactctagtggaccccgcctaaggccacacccctttgggccaggctccaccccctattctgtgggtaccttctagaacccccttcaaagtcagagcttttttttttttttttttggagacagtcttgctctctctcccaggctggagtgcagtggcgtgatctcggctcactgcaacctctgcctcccaggttcaagtgattctcgtgcctccacctcctgagtagctgggattacaggtgcgcgccaccacgcctggctaatttttgtgtctttagtagagacagggtttcaccttgttggccaggctggtctcaaactcccaacctcaggtgatccgcccacctcggcctcccagagtgctggggttacaggcgtgagccaccgcccccagcccaaagtcagagctctttataggagactctaacatgtaaccctgaccctggccctaactaagtcaattccaaaccccttcctgcctccagccctgaccccactcactgaggcctgaccccacttcttgagaccagttccatccctaaagccctggtctccctcccatccccaggctccagcccccacagctttggcactacccctgagcttgtccaggaatcctgtacccaattttaccctcacatgtagttctagccaattccaggaatctgtgaggtccagttagagtccagtaaccctacctgagcctgggctctgtccttgagcttgagcctgggcttgagaggtgccactcttattctccaggccctgcccctgccccctcagcatgtcagacacccaccctctagctggtctggcctcttgagtctgaaacccacccccagcccaagccccgcctctgagccccgcccaacccattttccgttcccagagcatgttctcgccaacaatgatgtttcctgtgaccacccctctaacaccgtgccctctgggagcaaccaggacctgggagctggggccggggaagacgcccggtcggatgacagcagcagccgcatcatcaatggatccgactgcgatatgcacacccagccgtggcaggccgcgctgttgctaaggcccaaccagctctactgcggggcggtgttggtgcatccacagtggctgctcacggccgcccactgcaggaagaagtgagtgggagttccaagaggagggttggtggggacggggaagtgggggtgggggtggggaagtgggggtgggggtgtcatggaggtgagggctggtggggacggggaagtggggttgggggtgtcatggaaggtgagggttggtggggatgggttggggatgtgggagcaggaggaggtcgagttggggataggactaaggatggagttttgcgggggagcaaggtgggaggatgaggttggagaggggagagtgttgtggtagggaatgggaaggagccaaggatgggttggatttggggttaggagcatatatttgttgaatggtttgggatggaggtggaattgggattggctttagaattgggggtgggtgaaaatcgggctggggtggaaatgaagatagcatggagatagggttgagattgggagcagatatagaatgaaggatggggattggagttttgggtggggttggagatggttggatttgggcttgagaatgcatatggtgatggcttctgggtagggaaagaattagggttgggaatgggatgggtttggaattgtgactgggatggggacaggcatgggattggagaccaagagggagttgaggatggtttggggaccgggggtggggatgggggtggggctggggctgggtgtggggttgggattggcgttggacgtggagatagagatcagggttggtggtgacctgccccatcttcctcagagttttcagagtccgtctcggccactactccctgtcaccagtttatgaatctgggcagcagatgttccagggggtcaaatccatcccccaccctggctactcccaccctggccactctaacgacctcatgctcatcaaactgaacagaagaattcgtcccactaaagatgtcagacccatcaacgtctcctctcattgtccctctgctgggacaaagtgcttggtgtctggctgggggacaaccaagagcccccaaggtgagtgtccaggttcttcttgataccgacccatctctgccgccttccatctttctccacttctcattgtgttcctgtttgacagtgcacttccctaaggtcctccagtgcttgaatatcagcgtgctaagtcagaaaaggtgcgaggatgcttacccgagacagatagatgacaccatgttctgcgccggtgacaaagcaggtagagactcctgccaggtgaggacacctctctttattcagcagatacacactgagtgccaactcggtaacatggagcgttgccaaattctgagaatccagcaattgccaagacagtcaggacccctgttctcacagagctcataccctagagtagtggtgtttagtagaaataatgctgagctgcttatgtcatttccagttttttagtagccacattaaaacaggtaaaaaaggctgggcgcagtggctcacacctgtaatcccagcactttgggaggctgaggcaggcagatcacctttggtcaggagtttgagactagcctggccaacatggcgaaactctgtctctaaaaaaaaatacaaaaattagcctggcatggtggcgggcgcctgtaatctcagctgctcaggaggccgagacacaagaatcacttaaacccaggaggtggaggttgcagtgagctgagatcgtgccactcactccaacctgggagacagagtgacacttttgtctcaaaaagaaaaaaaaaaacaagtaaaaaagaaacaggtgaagttaactttaataacccaatgtatcccaaatacaatcatttcaaagtgtaattaatataaaacaattatgaatgagatactttacattcttttcttgttttcatattaagtctttgaaagtgagtatatatgttatgctgacagcacatctcaatttggactagctacatttcaggtgctcagtagccacatgtggctagcagttactgtattggatggcacggatctagagggaaagatcagggctgttttgtatggttgggcaggttgtgcactgcataaagataccatatctaataggggcactccgtgttacagatgtcagttttggcagttttcaggcgtgtggtagttaagtgtcttgtttcaacaaaatctgtaatatgacagttttctagcaagtgctggtaaaatatcttgaggaaggaaaagagaaatctggtaggtatttttacaagagaatatttaatacaggggattaattgcaaagctgctggaagggctggaggaacaaagttaaaaaataaaaaactctgtggtcaagaatctgcataaatagggcaatttcagagagtggtaaaggttaaccccaaaataaaacatggttttaggatagtaaacaataagggccaatattcaaaaaggtggtcaggggagcctccttggagaggtggcatttgagcagagaatggatgacacaaagaagctaaactcgtgaagtttaaggggaaagaaaaggcacgtgcaaaggccctgaggcagtaaggaatttggctgattcaaagaagaagaggaaaccaatgcaactggagaacaaaagtgggggcaacagtagaaagtgacgctggaggtgtaggcaggggcgaatgctctgcaagtatttcttggtcaccaacacagagcttccctatgttctaatggaagctgtatctgttgaggaagacagaatttaaaatcaaactgttacatcaaccagcacccttctctgtattcaggctcccaagggatctagaaggacgtaagttaacaagctctcattagcagggtgtgtgtttcaacagtagttaggaagctggggattcaggagtactccagtcccatggctatgaaaagctccccccaaattgtacaaacctgacaaatgcaacacctccccagctctccccatttcttctctgtgccctgggtgtgggggggtgggttgcgagggggaaaacttttaacagaagaaagcacatctcggccgggcgtggtggctcacacctgtaatcccaacactttgggaggccgaggcgggtggatcactaggtcaggagatggagaccatcctggctgacacggtgaaaccctgtctctactaaaaacacaaaaaattagccgggcgtggtggcaggcgcctgtagtcccagctactcgggaggctgaggcaggagaatggcctgaacccgggaggcggaacttgcagtgagccgaggttgcaccactgcactccagcctgggcaacacagtgagactccgtctcaaaaaaaaaaaaagaaaagaaaagaaatcacatctcattcaagtggtggcatttaaaactatttagcctttctgtaggcaaggttagtatcttgtttttccagacctcaaggtgtttttttgtttgttttttcataccggtgtgtggtctgggtgtggccactaaaagctacaagcaagaaataataacaactacaacaatactaataccaatagtataaaaataatagcatctggctaattgctggacactgttttaagtggtttgcatgcctcagctcattaactcatttacctgttattattggccctattttacaaacaaggagccaaggctcagagcagttaactaacagcctctcaaaagaaactctgcagagatattaaatttaaaaaataatgagagaaattaaaccacaagaaagttgaaatttagaggtacaggcagctaagcttgtttgctttgaaacagtgtctgctactgggaaaaaggcaagtcttggctttcctaataattgataccaggactctgtaattcatattttgcatgcatgtaagtaagaaatgaagccgggtgcaatggcacatgccagtaatcccagcactctgggagactgaagtgggaagatcacttgagctcaggagttcaagaccagcctgggcaactaaaaattaaaaaaataaaaatactaattgtttttattttagtagattttattcataccacttacatcattattgtagtatgtacatatttatttcttttcttttcttttcttttcttttttgagacggagtctcgctctgtcacccaggctggagtgcaatggcaccatatcagctcactgcagcatgcgcctcctgggttcaagcatttcttccacctcagcctcccaagtagctgggataacaggcacccaccaccatgcctggctatttttttttttccgtagagatggggttccaccatgttggccaggctggtcttgaactcctgacctccagtgatctgcctgcctcggcctcccaaattgctggtattacaggtgtgagccaccgtgcccaggtgggagatagacatttctctctacctcaaacagaggtccactcaagctacttttcattttcttcataaatattagccgagtggctattttgcaccaggaatggttccaggtgctgtggatatggcatcaggcaaaacagaccaaaaacttcctgccgcgtggacctcatgttccccaagtggaagacaggcaataaagagatagataaatatgtagtaaattaaaaaaaaaaaaaattagccgggtgtggtggcttgcacctgtagttccagctacttgggaggctgaggtgggagaattgcttgagcccaaacgtttgaggctgcggtaagccatgactgcactgctgcactccagacagcagcctgggtgacaaagcaagacgtttttgtcagaaagaaaaaaaaaagagacgaagggaggaaggagagagaaaggaaggaaggaaggagaaagaaaggaaggaaggagaaagaaaggaaggaaggaaggagaaagaaaggaagaaagagaaagaaagaaaaagaaagaaagaaagaagaaagaaaagagagaggaaggaaggaaagaaggaaaagagggaaaaaaatgactgttgaagagcagtgagtattattataggagggtaattatagggaggtatggggaattgaagacaggaaacacaaattagtccaagcgaatggatttctattgggagtgattctgcccctagaagacactggcaataccaggagacatttttggttgtcacaactatatggaggggcattactggcaactaatggatagatgccaagtgtgctgttcaacatgctatgatgcacacggcaggcctccacaacaaaccattatccagcttcagatgcccacagtgcccagatcgaggaaccctcatccaggggctgagaaccgtatttttgcagaagggaggtataaggatgggttggtggagaatggggaaggaaggtgtgtgtccagtaagagaaataaggcctgcacaggctggaggggagagtgagagagaaagggaggcggagagatacacgatgagggagacaggctggaacagaaagtagagacgaagattcgagatgtggagaggaagggtcacagacccccccgaaatgatgtgtggacaacaggaatctggaagaggaagatggagtggagagtgacaaatggggtctaaaggttgaacttggaggccaggcatggtggctcacgcctgtaatcccaacactttggaggctgaggtgggcgaatcacttgaggccaggagttcgagaccagcctggccaacatggtgaaaccccgtctctacaaaaaaaatacaaaaaattagccgggtgtggtgatggacacctgtagtcacagctacttgggaggctgaggcaggagaattgcttgaacccgggagatggaggctgcagtgagctgaggtcaggccactgcgctccaacctgggcaacagagtaagactccatctcaaaaaaaaaaaagctggatttggagtgaaatattaataacattctccctctctctccttttgcctgtgtctccatctctgtctttttctgcatttcttcatctctgtactttccatctctgtgtgtctgttcccatctgcttctccatctatgggcatctctgggtctctcatgtctccttctgcccactttgccacatctctgcctctctcatgcccccctttctctcctgcagggtgattctggggggcctgtggtctgcaatggctccctgcagggactcgtgtcctggggagattacccttgtgcccggcccaacagaccgggtgtctacacgaacctctgcaagttcaccaagtggatccaggaaaccatccaggccaactcctgagtcatcccaggactcagcacaccggcatccccacctgctgcagggacagccctgacactcctttcagaccctcattccttcccagagatgttgagaatgttcatctctccagcccctgaccccatgtctcctggactcagggtctgcttcccccacattgggctgaccgtgtctctctagttgaaccctgggaacaatttccaaaactgtccagggcgggggttgcgtctcaatctccctggggcactttcatcctcaagctcagggcccatcccttctctgcagctctgacccaaatttagtcccagaaataaactgagaagtgg

SEQ ID NO: 4 - DNA, 314 bp, Human
tcaggcccggtgcccagagcccaggaggaggcagtggccaggaaggcacaggcctgagaagtctgcggctgagctgggagcaaatcccccaccccctacctgggggacagggcaagtgagacctggtgagggtggctcagcaggaaggaaggagaggtgtctgtgcgtcctgcacccacatctttctctgtcccctccttgccctgtctggaggctgctagactcctatcttctgaattctatagtgcctgggtctcagcgcagtgccgatggtggcccgtccttgtggttcctctctacttggggaaatcagg

SEQ ID NO: 5 - DNA, 242 bp, Human
gctgggagcaaatcccccaccccctacctgggggacagggcaagtgagacctggtgagggtggctcagcaggaaggaaggagaggtgtctgtgcgtcctgcacccacatctttctctgtcccctccttgccctgtctggaggctgctagactcctatcttctgaattctatagtgcctgggtctcagcgcagtgccgatggtggcccgtccttgtggttcctctctacttggggaaatcagg

SEQ ID NO: 6 - DNA, 1222 bp, Human
  5′UTR (1)..(14)
  CDS (15)..(896)
  3′UTR (897)..(1222)
caggtgcagcggccatggctacagcaagacccccctggatgtgggtgctctgtgctctgatcacagccttgcttctgggggtcacagagcatgttctcgccaacaatgatgtttcctgtgaccacccctctaacaccgtgccctctgggagcaaccaggacctgggagctggggccggggaagacgcccggtcggatgacagcagcagccgcatcatcaatggatccgactgcgatatgcacacccagccgtggcaggccgcgctgttgctaaggcccaaccagctctactgcggggcggtgttggtgcatccacagtggctgctcacggccgcccactgcaggaagaaagttttcagagtccgtctcggccactactccctgtcaccagtttatgaatctgggcagcagatgttccagggggtcaaatccatcccccaccctggctactcccaccctggccactctaacgacctcatgctcatcaaactgaacagaagaattcgtcccactaaagatgtcagacccatcaacgtctcctctcattgtccctctgctgggacaaagtgcttggtgtctggctgggggacaaccaagagcccccaagtgcacttccctaaggtcctccagtgcttgaatatcagcgtgctaagtcagaaaaggtgcgaggatgcttacccgagacagatagatgacaccatgttctgcgccggtgacaaagcaggtagagactcctgccagggtgattctggggggcctgtggtctgcaatggctccctgcagggactcgtgtcctggggagattacccttgtgcccggcccaacagaccgggtgtctacacgaacctctgcaagttcaccaagtggatccaggaaaccatccaggccaactcctgagtcatcccaggactcagcacaccggcatccccacctgctgcagggacagccctgacactcctttcagaccctcattccttcccagagatgttgagaatgttcatctctccagcccctgaccccatgtctcctggactcagggtctgcttcccccacattgggctgaccgtgtctctctagttgaaccctgggaacaatttccaaaactgtccagggcgggggttgcgtctcaatctccctggggcactttcatcctcaagctcagggcccatcccttctctgcagctctgacccaaatttagtcccagaaataaactgagaagtgg

SEQ ID NO: 7 - protein, 293 aa, Human; identical to SEQ ID NO: 2

SEQ ID NO: 8 - DNA, 1532 bp, Human
  5′UTR (1)..(324)
  (CDS) (325)..(1206)
  3′UTR (1207)..(1532)
tcaggcccggtgcccagagcccaggaggaggcagtggccaggaaggcacaggcctgagaagtctgcggctgagctgggagcaaatcccccaccccctacctgggggacagggcaagtgagacctggtgagggtggctcagcaggaaggaaggagaggtgtctgtgcgtcctgcacccacatctttctctgtcccctccttgccctgtctggaggctgctagactcctatcttctgaattctatagtgcctgggtctcagcgcagtgccgatggtggcccgtccttgtggttcctctctacttggggaaatcaggtgcagcggccatggctacagcaagacccccctggatgtgggtgctctgtgctctgatcacagccttgcttctgggggtcacagagcatgttctcgccaacaatgatgtttcctgtgaccacccctctaacaccgtgccctctgggagcaaccaggacctgggagctggggccggggaagacgcccggtcggatgacagcagcagccgcatcatcaatggatccgactgcgatatgcacacccagccgtggcaggccgcgctgttgctaaggcccaaccagctctactgcggggcggtgttggtgcatccacagtggctgctcacggccgcccactgcaggaagaaagttttcagagtccgtctcggccactactccctgtcaccagtttatgaatctgggcagcagatgttccagggggtcaaatccatcccccaccctggctactcccaccctggccactctaacgacctcatgctcatcaaactgaacagaagaattcgtcccactaaagatgtcagacccatcaacgtctcctctcattgtccctctgctgggacaaagtgcttggtgtctggctgggggacaaccaagagcccccaagtgcacttccctaaggtcctccagtgcttgaatatcagcgtgctaagtcagaaaaggtgcgaggatgcttacccgagacagatagatgacaccatgttctgcgccggtgacaaagcaggtagagactcctgccagggtgattctggggggcctgtggtctgcaatggctccctgcagggactcgtgtcctggggagattacccttgtgcccggcccaacagaccgggtgtctacacgaacctctgcaagttcaccaagtggatccaggaaaccatccaggccaactcctgagtcatcccaggactcagcacaccggcatccccacctgctgcagggacagccctgacactcctttcagaccctcattccttcccagagatgttgagaatgttcatctctccagcccctgaccccatgtctcctggactcagggtctgcttcccccacattgggctgaccgtgtctctctagttgaaccctgggaacaatttccaaaactgtccagggcgggggttgcgtctcaatctccctggggcactttcatcctcaagctcagggcccatcccttctctgcagctctgacccaaatttagtcccagaaataaactgagaagtgg

SEQ ID NO: 9 - DNA, 1329 bp, Human
  5′UTR (1)..(121)
  (CDS) (122)..(1003)
  3′UTR (1004)..(1329)
tcaggcccggtgcccagagcccaggaggaggcagtggccaggaaggcacaggcctgagaagtctgcggctgagctgggagcaaatcccccaccccctacctgggggacaggtgcagcggccatggctacagcaagacccccctggatgtgggtgctctgtgctctgatcacagccttgcttctgggggtcacagagcatgttctcgccaacaatgatgtttcctgtgaccacccctctaacaccgtgccctctgggagcaaccaggacctgggagctggggccggggaagacgcccggtcggatgacagcagcagccgcatcatcaatggatccgactgcgatatgcacacccagccgtggcaggccgcgctgttgctaaggcccaaccagctctactgcggggcggtgttggtgcatccacagtggctgctcacggccgcccactgcaggaagaaagttttcagagtccgtctcggccactactccctgtcaccagtttatgaatctgggcagcagatgttccagggggtcaaatccatcccccaccctggctactcccaccctggccactctaacgacctcatgctcatcaaactgaacagaagaattcgtcccactaaagatgtcagacccatcaacgtctcctctcattgtccctctgctgggacaaagtgcttggtgtctggctgggggacaaccaagagcccccaagtgcacttccctaaggtcctccagtgcttgaatatcagcgtgctaagtcagaaaaggtgcgaggatgcttacccgagacagatagatgacaccatgttctgcgccggtgacaaagcaggtagagactcctgccagggtgattctggggggcctgtggtctgcaatggctccctgcagggactcgtgtcctggggagattacccttgtgcccggcccaacagaccgggtgtctacacgaacctctgcaagttcaccaagtggatccaggaaaccatccaggccaactcctgagtcatcccaggactcagcacaccggcatccccacctgctgcagggacagccctgacactcctttcagaccctcattccttcccagagatgttgagaatgttcatctctccagcccctgaccccatgtctcctggactcagggtctgcttcccccacattgggctgaccgtgtctctctagttgaaccctgggaacaatttccaaaactgtccagggcgggggttgcgtctcaatctccctggggcactttcatcctcaagctcagggcccatcccttctctgcagctctgacccaaatttagtcccagaaataaactgagaagtgg

SEQ ID NO: 10 - DNA, 9729 bp, Human
  mRNA (1388)..(1431): Exon 1
taccacattttcttaatccagtctatcactgatggacatttaggttgattccctgtgtttgctgttgtcaatagttctacaatgaacgtacgtgtccatgtgtctttaaacagaatgatttatattcctttgggtacacacactggggcttatgagagggtggagagtgggaggaaggagaggatcagaaaaaaataactaatgggtactaggcttaatacctgggtgattaaataatctgtataacaaacccccatggcgcacgttcacctacgcaacaaacctgcacatcctgcacatgtacccccgaactgaaaagttaaaaaaagaaaaataaatatttgcttataaattaataaatgaagccctcaaaaatgttctattagataatgttaagtacagacatttttgttataaatacataatatacaaagaaatctatgtataacatgattaaaatgaccataagaacatagatcctaaacatggcaaatattagtggggtggggttagggaaagcgttgtttttaacttacacctctctgttagagttgggaatgggttcaggcgtaattacaggcacgactgggatcagcttggacaagttcccccaggcgggccagaattaggatgtagggtctaggccacccctgagagggggtgagggcaagaaaatggccccagaagccgggcgcagtggctcacgcctgtaatcccagcactttgcggggccgaggcgggcacatcatgaggtcaggagatcgagaccattctggccaacatagtgaaacccggtctctactaaaaatacaaaaattatctgggagtggtggtgcgtgcctgtaatcccaggtactcgggaggctgaggcaggagaatcacttgaacctgggaggcggagctggcagtgagccgagatcgcgccaccgcactccagcctggcgatagagagagactccatccaaaaaaaagaaaggaagggagggagggaggagggaagaaagaaagaaaaccgccccagagaaggacccgagccagagcctattctctgagctcagcgactgcttgaatcccgctcctgcccctcagacccagcgcaccgggtccctcccccgagagcagccaggagggactgtgggaccagaatgtgcgggggcgcaggagctgggcaccgcccgtccttcggagggagggtggagagagagtgcagtggtgccaattgctctcgctgcgtcagggttccagataaccagaaccgcaaatgcaggcgggggtgtcccagagtcggctccgcctgcaccccagggcgctggggccgggcatggggcggggggtgatataagaggacggcccagcagagggctgaagattttggagcccagctgtgtgccagcccaagtcggaacttggatcacatcagatcctctcgaggtgagaagaggcttcatcaagggtgcacctgtaggggagggggtgatgctggctccaagcctgactctgctctcgagaggtaggggctgcagcctagactcccggtcctgagcagtgagggcctggaagtctgcaatttggggccttttagggaaaaacgaactacagagtcagaagtttgggttccacagggaagggcaagatcggagcctagattcctgggtctctagggatctgaagaacaggaattttgggtctgagggaggaggggctggggttctggactcctgggtctgagggaggagggcctgggggcctggactcctgggtctgagggaggaggggctgggggtctcgactcctgggtctgagggaggaggggctgggggcctggactcctgggtctgagggaggaggggctgggacctggactcctaggtctgagggaggaggagctggggcctggactcctgggtctgagggaggaggggctggggcctggactcctgggtctgagggaggatgggctgaggcctagactcctgggtctgagggaggaggggctggggcctggactcctgggtctgagggaggaggggctggagcctggactcctgggcctgagggaggagggactgagacctggactcctaggtctgagggaggagggactgggacctggactcctgggtctgagggaggaggagctgggggcctggactcctgggtctgagggaggcggggctgggggcctggactcctgggtctgagggaggaggggttggggcctggactcctgagcctgagggaggagggacttggacctggactcctaggtctgagggaggaggagctgggggcctggactcctaggtctgagggaggaggggctgggggcctggactcctgggtctgagggaggaaggtgctagggtctggactcttgggtatgagggaggaggaggttaggggtctggacttctgagtgtaaggaaggagaggccagagaaaggaatttctgggtctgagggaggaggggctggggttctggacccctaggtctgagggaggaggggctggggcctggactcctgggtctgtggggggaggggctggggcctggacccctgggtctgagtggggaggggctgggcctgaatgctttctccttctcagctccagcaggagaggcccttcctcgcctggcagcccctgagcggctcagcagggcaccatggcaagatcccttctcctgcccctgcagatcctactgctatccttagccttggaaactgcaggagaagaaggtgaaagctggactgggaagtctgacctcacctcagggcccccactgaccctctccaaggagtccctgagtcagaacccttccctcctcaaacagcttccatcctgggaggaccagactgtcggctgaagcccccgctcttcctgcttctgctgactcagggggtctctgtcccctccaggccctgcctcctgtgctcagggtctctctgtggttccccagatgagatgcgcctcctgggtttctgagtgggctccttctgtctgtctctatccctatctcttgctttctctgtatttctccacacattttcatctgtctctgtccatctctgactctgggaatccctgaggtgcagcctcagccttcccctaatgctagctacccacatgctcctccatgtctccatccagcccagggtgacaagattattgatggcgccccatgtgcaagaggctcccacccatggcaggtggccctgctcagtggcaatcagctccactgcggaggcgtcctggtcaatgagcgctgggtgctcactgccgcccactgcaagatgaagtaggtgccacccaagtctctgctggaggtgcgccagcatctccagctcgctatgggggtggaagggcagtctttctgtgcctacggctctattctcctctctctgggtctctgtccccctctctctgggcctctgtaccccctctccctggggctctgtccccctctctccctggctctctgtctccctctctctgggtctctgtccccctctctctggatctctgttcccctctctctgtgtctctgtcccccattctctctaggtctctgttccccctcctctctctctgggtctctgtccctctctctctggtctctgtccccctctctctctggatctctgtccccctctccctgggcctctgtaccccctctccctggggctctgtcccccctctctgggtctctgtctgcctttctctctggatctctgttcccctctgtgtctctgtccccctctctctctgggtctctgttccccctcctctctttctgggtctctgtccctctctctctgggtctctgtccccctctctctctggtctctgttccccctcctctctctctggtctctgtccctctctctctgggtctctgtcaccctctctctctgggtctctgtcaccctctctctctggtctctgttccccctcctctctctgtgggtctctgtccctctctctctgggtctctgttcccctctctctctggtctctgttccccctcctctctctccggatctctgtccccctctccctggggctctgtccccctctctccctggctctctgtcttcctctctctggggctctgtccccctctctctctggtctctgttcccctctctctgggtctctgtccctctctctctgggtctctgtccctctctctctggatctctgtccccctctccctgggcctctgtaccccctctccctggggctctgtccccctctctctgggtctctgtctgcctttctctctggatctctgttcccctctgtgtctctgtccccctctctctctgggtctctgttccccctcctctctttctgggtctctgtccctctctctctgggtctctgtccccctctctctctggtctctgttccccctcctctctctctggtctctgtccctctctctctgggtctctgtcaccctctctctctgggtctctgtcaccctctctctctggtctctgttccccctcctctctctgtgggtctctgtccctctctctctgggtctctgttcccctctctctctggtctctgttccccctcctctctctccggatctctgtccccctctccctggggctctgtccccctctctccctggctctctgtcttcctctctctggggctctgtccccctctctctctggtctctgttcccctctctctgggtctctgtccctctctctctgggtctctgtccctctctctctggatctctgtccccctctctctctgggtctctgttcccctctctctgggtctctgtcccctctcctctctctgtgtctctctccccctcctctctctgtgtctctgtcccccctcctatctctgtgtctctctcccccctcctctctctgggtctctgtccccccctctctgggtctctgtctccctctctctggggctctgtccccctctctctctggatctctgttcccctctctctgggtctctgtctcccctcctctctctgtgtctctgtcccccctcctctctctgggtctctgtccccaccccgtcccccaggtctttgcacaccctctctgtcacagtgtctcttctgaatctgtgaatgtcactcctcgcagtgagtacaccgtgcacctgggcagtgatacgctgggcgacaggagagctcagaggatcaaggcctcgaagtcattccgccaccccggctactccacacagacccatgttaatgacctcatgctcgtgaagctcaatagccaggccaggctgtcatccatggtgaagaaagtcaggctgccctcccgctgcgaaccccctggaaccacctgtactgtctccggctggggcactaccacgagcccagatggtaggtggcctcagtgacccaggagtgcaggccccagccctcctccctcagacccaggagtccaggcccccagcccctcctccctcagacccaggagtccaggcctcagcccctcctccctcagacccaggagtccaggcccccagcccctcctccctcagacccgcgagtccagaccccagcccctcctccctcagacccagcagtcctgggccccagaccctcctccctcggaaccaggagcctgaacaacagcccttctggtcctcgcccccatcctctctgactgacagctctccctgctcctccctgcagtgacctttccctctgacctcatgtgcgtggatgtcaagctcatctccccccaggactgcacgaaggtttacaaggacttactggaaaattccatgctgtgcgctggcatccccgactccaagaaaaacgcctgcaatgtgagaccctcccccccaattcctccccagtcctgggtaccctgtctgcatgccccagggacagagcttgacccaagtgactgggtaccaagcccggccttgccctccccccaggcctggcctcctcagctttttccacctcattctctgcctaggtcaggggtgggagtttacttaggggccgatgtggccctggggatgggacagagagtttaataggggtgagaaagtgggggtgggaccagggaaggagactgaggtgctggcctcaggcccaaaccctaagggggcaccaaaaacctcagtgattgagataaatcataatgcaatatttaaaaataaaaataaaaactcatgcagaagtccatgatggacaaaatgtcacattttaaataaagagcaggtggatcttactgaattttcccttgccgtaagtactagcgtggctcagcacagcgctgtactggcactgtcttcatttaaaatgtggataccatgcccatcatgcagttttatgtattacatttgatttcgttaagtactgcattgaagtattgtgtattgcagttactgagattttgtgcctgaagctgatgactcactcacctgaccctggccctggtcccggggaaaacactctttctctccacctcctctctgttccctctttctggccttttgtcatcccctctgtttctgaacagtcttcccacatctctctttgtgacataatttcatttcattcttttcctctttgttttttctctgtgttgagctagcttgctctccctcccttgttctctctccatgccctcctctctgctctctgtcttctccctctttctcttgcttctctctctctcctcccctccctctctcctctccctgcccccctgctctctcttttttcctctctctctgtctcctctctggccctctcctctttctctctctcccccacttctctgtctctcttcatctctctccctcatctctccttgccccctcctttttactgtctctctctttctctttcttctatctctctcctctccccgccgctcccccatctctgtctttctttctctctctttattctcctcctctcttccagtctctctctcctctccccacccccaccccatctctctccccacaccttccccccctttctctttgtctctctcttctacctctttcttctccacccccatctctctctctcttctcttcccacaccctccccatctccctcatctctttgtctgtctctcttctccctccttcttttccacccccatctctctgtctctctctctccccataccctttccctcttcctcatctctctttgtctctctctcctttccctctttcttctccacctccaactctctctgtctctccacacccatcctccttgctcacatctgcaccttcagctgtcaggggatgtgggatggtgagtgttagggatagaggagatgggagagagatgactgtcctagagaatagggtgttccccttctcccctggtgagggccagtttcatgaatgtgcaagctctgcacggacacagagccccacactcagaagggtctcaaacttagtctaatgcattcctgctgttgtcttgaaattctcaataatttttgaacaaagggccctgcattttcgttttgcaccaagtcctgtaaattatgtaactggtcttcaccctggtctccgagaccatcgtgtccccctttcctgcgccacagggcacgcatccaccccttggagatgatgttccttctcccactagcttggagcagggtccttaacattggaaaataaagagtgctctgatcctggaagccccaccccttctctgcaattggtctcattggccaagggtcaaaccagtgtcttcaaaggacctagtgtgtccctagcactagctctcccattagtccccagagacaatgagtctcttctcattggctatggtggaagtccataatctgcaagacaaagaccgataactgaggaatgtatgagaatgagttgggctttgatctgaagccaaagttaatctccggctctattccctctagggtgactcagggggaccgttggtgtgcagaggtaccctgcaaggtctggtgtcctggggaactttcccttgcggccaacccaatgacccaggagtctacactcaagtgtgcaagttcaccaagtggataaatgacaccatgaaaaagcatcgctaacgccacactgagttaattaactgtgtgcttccaacagaaaatgcacaggagtgaggacgccgatgacctatgaagtcaaatttgactttacctttcctcaaagatatatttaaacctcatgccctgttgataaaccaatcaaattggtaaagacctaaaaccaaaacaaataaagaaacacaaaaccctcagtgctggagaagagtcagtgagaccagcactctcaaacactggaactggacgttcgtacagtctttacggaagacacttggtcaacgtacaccgagacccttattcaccacctttgacccagtaactctaatcttaggaagaacctactgaaacaaaaaaaatccaaaatgtagaacaagacttgaatttaccatgatattatttatcacagaaatgaagtgaaaccatcaaacatgttccaaaagtaccagatggcttaaataatagtctggcttggcacaacgatgttttttttctttgagacagagtctctgttgcttgggctgcaatgcagtgatgcaatcttggctcactgcaacctccgcctcctgggttcaagtgattctcgtgcttcagcctcccaagtacctgggactacaggtgtgcaccaccacaccaggctaattttttgtgtatttttactagagacagggtttcaccatgttggccagcgtggtcttgaacgcctgacctcagatgatccacccaccttggcctcccaaagtgctgggattacaggcatgagccaccacggccagcccacaatgatattacaaacctattaaaaatgatacttagacagaattgtcagtattattcaagaacatttaggctataggatgttaaatgacaaaaggaaggacaaaaatatatatgtatgtgaccctacccataaaaaatgaaatattcacagaatcagatctgaaaacacatgtcccagactgcatactggggtcgtcatgaggtgtctccttccttctgtgtacttttccttgaatgtgcacttttataacatgaaaaataaaggtggggaaaaaagtctgaagatctaagattggagagaggtgacctttcaggaagggagactagaaagaaatatgtgcctggttttgagccctggtcctgccggccctgttccagggcatatttccatttcccagatctcagtttttcctgtctgtaaaatgggagagagaggaaaggatggagagaggaagaaggaagggaggagggaggagagaacaggccaacttcatcagcgtgggaaggggtgtgaaagtgtttctgagcatctcacgagtgacaagtgaggagggaggctggcggttttcagagggattgggatgacagtagacaggacacaggggtcccacaggggtctgccagaagtaagcaaacagtgccggaggaagatggtggcacctgctccccaagaagggagggaaaggaacctcgggaagcgggtaggatgagggaggagtcctctgtgactcagagcctggccacagccccagccatctaacatcaaagatcctctgtgtggtcacacctcagacgctgctgaccgaggagccactccagcccaggacacgccctcctacctgttcttcctgtttttctcccagaattc

SEQ ID NO: 11 - protein, 253 aa, Human
MARSLLLPLQILLLSLALETAGEEAQGDKIIDGAPCARGSHPWQVALLSGNQLHCGGVLVNERWVLTAAHCKMNEYTVHLGSDTLGDRRAQRIKASKSFRHPGYSTQTHVNDLMLVKLNSQARLSSMVKKVRLPSRCEPPGTTCTVSGWGTTTSPDVTFPSDLMCVDVKLISPQDCTKVYKDLLENSMLCAGIPDSKKNACNGDSGGPLVCRGTLQGLVSWGTFPCGQPNDPGVYTQVCKFTKWINDTMKKHR

SEQ ID NO: 12 - DNA, 8831 bp, Human
  mRNA (1244)..(1431): Exon 1
taccacattttcttaatccagtctatcactgatggacatttaggttgattccctgtgtttgctgttgtcaatagttctacaatgaacgtacgtgtccatgtgtctttaaacagaatgatttatattcctttgggtacacacactggggcttatgagagggtggagagtgggaggaaggagaggatcagaaaaaaataactaatgggtactaggcttaatacctgggtgattaaataatctgtataacaaacccccatggcgcacgttcacctacgcaacaaacctgcacatcctgcacatgtacccccgaactgaaaagttaaaaaaagaaaaataaatatttgcttataaattaataaatgaagccctcaaaaatgttctattagataatgttaagtacagacatttttgttataaatacataatatacaaagaaatctatgtataacatgattaaaatgaccataagaacatagatcctaaacatggcaaatattagtggggtggggttagggaaagcgttgtttttaacttacacctctctgttagagttgggaatgggttcaggcgtaattacaggcacgactgggatcagcttggacaagttcccccaggcgggccagaattaggatgtagggtctaggccacccctgagagggggtgagggcaagaaaatggccccagaagccgggcgcagtggctcacgcctgtaatcccagcactttgcggggccgaggcgggcacatcatgaggtcaggagatcgagaccattctggccaacatagtgaaacccggtctctactaaaaatacaaaaattatctgggagtggtggtgcgtgcctgtaatcccaggtactcgggaggctgaggcaggagaatcacttgaacctgggaggcggagctggcagtgagccgagatcgcgccaccgcactccagcctggcgatagagagagactccatccaaaaaaaagaaaggaagggagggagggaggagggaagaaagaaagaaaaccgccccagagaaggacccgagccagagcctattctctgagctcagcgactgcttgaatcccgctcctgcccctcagacccagcgcaccgggtccctcccccgagagcagccaggagggactgtgggaccagaatgtgcgggggcgcaggagctgggcaccgcccgtccttcggagggagggtggagagagagtgcagtggtgccaattgctctcgctgcgtcagggttccagataaccagaaccgcaaatgcaggcgggggtgtcccagagtcggctccgcctgcaccccagggcgctggggccgggcatggggcggggggtgatataagaggacggcccagcagagggctgaagattttggagcccagctgtgtgccagcccaagtcggaacttggatcacatcagatcctctcgaggtgagaagaggcttcatcaagggtgcacctgtaggggagggggtgatgctggctccaagcctgactctgctctcgagaggtaggggctgcagcctagactcccggtcctgagcagtgagggcctggaagtctgcaatttggggccttttagggaaaaacgaactacagagtcagaagtttgggttccacagggaagggcaagatcggagcctagattcctgggtctctagggatctgaagaacaggaattttgggtctgagggaggaggggctggggttctggactcctgggtctgagggaggagggcctgggggcctggactcctgggtctgagggaggaggggctgggggtctcgactcctgggtctgagggaggaggggctgggggcctggactcctgggtctgagggaggaggggctgggacctggactcctaggtctgagggaggaggagctggggcctggactcctgggtctgagggaggaggggctggggcctggactcctgggtctgagggaggatgggctgaggcctagactcctgggtctgagggaggaggggctggggcctggactcctgggtctgagggaggaggggctggagcctggactcctgggcctgagggaggagggactgagacctggactcctaggtctgagggaggagggactgggacctggactcctgggtctgagggaggaggagctgggggcctggactcctgggtctgagggaggcggggctgggggcctggactcctgggtctgagggaggaggggttggggcctggactcctgagcctgagggaggagggacttggacctggactcctaggtctgagggaggaggagctgggggcctggactcctaggtctgagggaggaggggctgggggcctggactcctgggtctgagggaggaaggtgctagggtctggactcttgggtatgagggaggaggaggttaggggtctggacttctgagtgtaaggaaggagaggccagagaaaggaatttctgggtctgagggaggaggggctggggttctggacccctaggtctgagggaggaggggctggggcctggactcctgggtctgtggggggaggggctggggcctggacccctgggtctgagtggggaggggctgggcctgaatgctttctccttctcagctccagcaggagaggcccttcctcgcctggcagcccctgagcggctcagcagggcaccatggcaagatcccttctcctgcccctgcagatcctactgctatccttagccttggaaactgcaggagaagaaggtgaaagctggactgggaagtctgacctcacctcagggcccccactgaccctctccaaggagtccctgagtcagaacccttccctcctcaaacagcttccatcctgggaggaccagactgtcggctgaagcccccgctcttcctgcttctgctgactcagggggtctctgtcccctccaggccctgcctcctgtgctcagggtctctctgtggttccccagatgagatgcgcctcctgggtttctgagtgggctccttctgtctgtctctatccctatctcttgctttctctgtatttctccacacattttcatctgtctctgtccatctctgactctgggaatccctgaggtgcagcctcagccttcccctaatgctagctacccacatgctcctccatgtctccatccagcccagggtgacaagattattgatggcgccccatgtgcaagaggctcccacccatggcaggtggccctgctcagtggcaatcagctccactgcggaggcgtcctggtcaatgagcgctgggtgctcactgccgcccactgcaagatgaagtaggtgccacccaagtctctgctggaggtgcgccagcatctccagctcgctatgggggtggaagggcagtctttctgtgcctacggctctattctcctctctctgggtctctgtccccctctctctgggcctctgtaccccctctccctggggctctgtccccctctctccctggctctctgtctccctctctctgggtctctgtccccctctctctggatctctgttcccctctctctgtgtctctgtcccccattctctctaggtctctgttccccctcctctctctctgggtctctgtccctctctctctggtctctgtccccctctctctctggatctctgtccccctctccctgggcctctgtaccccctctccctggggctctgtcccccctctctgggtctctgtctgcctttctctctggatctctgttcccctctgtgtctctgtccccctctctctctgggtctctgttccccctcctctctttctgggtctctgtccctctctctctgggtctctgtccccctctctctctggtctctgttccccctcctctctctctggtctctgtccctctctctctgggtctctgtcaccctctctctctgggtctctgtcaccctctctctctggtctctgttccccctcctctctctgtgggtctctgtccctctctctctgggtctctgttcccctctctctctggtctctgttccccctcctctctctccggatctctgtccccctctccctggggctctgtccccctctctccctggctctctgtcttcctctctctggggctctgtccccctctctctctggtctctgttcccctctctctgggtctctgtccctctctctctgggtctctgtccctctctctctggatctctgtccccctctccctgggcctctgtaccccctctccctggggctctgtccccctctctctgggtctctgtctgcctttctctctggatctctgttcccctctgtgtctctgtccccctctctctctgggtctctgttccccctcctctctttctgggtctctgtccctctctctctgggtctctgtccccctctctctctggtctctgttccccctcctctctctctggtctctgtccctctctctctgggtctctgtcaccctctctctctgggtctctgtcaccctctctctctggtctctgttccccctcctctctctgtgggtctctgtccctctctctctgggtctctgttcccctctctctctggtctctgttccccctcctctctctccggatctctgtccccctctccctggggctctgtccccctctctccctggctctctgtcttcctctctctggggctctgtccccctctctctctggtctctgttcccctctctctgggtctctgtccctctctctctgggtctctgtccctctctctctggatctctgtccccctctctctctgggtctctgttcccctctctctgggtctctgtcccctctcctctctctgtgtctctctccccctcctctctctgtgtctctgtcccccctcctatctctgtgtctctctcccccctcctctctctgggtctctgtccccccctctctgggtctctgtctccctctctctggggctctgtccccctctctctctggatctctgttcccctctctctgggtctctgtctcccctcctctctctgtgtctctgtcccccctcctctctctgggtctctgtccccaccccgtcccccaggtctttgcacaccctctctgtcacagtgtctcttctgaatctgtgaatgtcactcctcgcagtgagtacaccgtgcacctgggcagtgatacgctgggcgacaggagagctcagaggatcaaggcctcgaagtcattccgccaccccggctactccacacagacccatgttaatgacctcatgctcgtgaagctcaatagccaggccaggctgtcatccatggtgaagaaagtcaggctgccctcccgctgcgaaccccctggaaccacctgtactgtctccggctggggcactaccacgagcccagatggtaggtggcctcagtgacccaggagtgcaggccccagccctcctccctcagacccaggagtccaggcccccagcccctcctccctcagacccaggagtccaggcctcagcccctcctccctcagacccaggagtccaggcccccagcccctcctccctcagacccgcgagtccagaccccagcccctcctccctcagacccagcagtcctgggccccagaccctcctccctcggaaccaggagcctgaacaacagcccttctggtcctcgcccccatcctctctgactgacagctctccctgctcctccctgcagtgacctttccctctgacctcatgtgcgtggatgtcaagctcatctccccccaggactgcacgaaggtttacaaggacttactggaaaattccatgctgtgcgctggcatccccgactccaagaaaaacgcctgcaatgtgagaccctcccccccaattcctccccagtcctgggtaccctgtctgcatgccccagggacagagcttgacccaagtgactgggtaccaagcccggccttgccctccccccaggcctggcctcctcagctttttccacctcattctctgcctaggtcaggggtgggagtttacttaggggccgatgtggccctggggatgggacagagagtttaataggggtgagaaagtgggggtgggaccagggaaggagactgaggtgctggcctcaggcccaaaccctaagggggcaccaaaaacctcagtgattgagataaatcataatgcaatatttaaaaataaaaataaaaactcatgcagaagtccatgatggacaaaatgtcacattttaaataaagagcaggtggatcttactgaattttcccttgccgtaagtactagcgtggctcagcacagcgctgtactggcactgtcttcatttaaaatgtggataccatgcccatcatgcagttttatgtattacatttgatttcgttaagtactgcattgaagtattgtgtattgcagttactgagattttgtgcctgaagctgatgactcactcacctgaccctggccctggtcccggggaaaacactctttctctccacctcctctctgttccctctttctggccttttgtcatcccctctgtttctgaacagtcttcccacatctctctttgtgacataatttcatttcattcttttcctctttgttttttctctgtgttgagctagcttgctctccctcccttgttctctctccatgccctcctctctgctctctgtcttctccctctttctcttgcttctctctctctcctcccctccctctctcctctccctgcccccctgctctctcttttttcctctctctctgtctcctctctggccctctcctctttctctctctcccccacttctctgtctctcttcatctctctccctcatctctccttgccccctcctttttactgtctctctctttctctttcttctatctctctcctctccccgccgctcccccatctctgtctttctttctctctctttattctcctcctctcttccagtctctctctcctctccccacccccaccccatctctctccccacaccttccccccctttctctttgtctctctcttctacctctttcttctccacccccatctctctctctcttctcttcccacaccctccccatctccctcatctctttgtctgtctctcttctccctccttcttttccacccccatctctctgtctctctctctccccataccctttccctcttcctcatctctctttgtctctctctcctttccctctttcttctccacctccaactctctctgtctctccacacccatcctccttgctcacatctgcaccttcagctgtcaggggatgtgggatggtgagtgttagggatagaggagatgggagagagatgactgtcctagagaatagggtgttccccttctcccctggtgagggccagtttcatgaatgtgcaagctctgcacggacacagagccccacactcagaagggtctcaaacttagtctaatgcattcctgctgttgtcttgaaattctcaataatttttgaacaaagggccctgcattttcgttttgcaccaagtcctgtaaattatgtaactggtcttcaccctggtctccgagaccatcgtgtccccctttcctgcgccacagggcacgcatccaccccttggagatgatgttccttctcccactagcttggagcagggtccttaacattggaaaataaagagtgctctgatcctggaagccccaccccttctctgcaattggtctcattggccaagggtcaaaccagtgtcttcaaaggacctagtgtgtccctagcactagctctcccattagtccccagagacaatgagtctcttctcattggctatggtggaagtccataatctgcaagacaaagaccgataactgaggaatgtatgagaatgagttgggctttgatctgaagccaaagttaatctccggctctattccctctagggtgactcagggggaccgttggtgtgcagaggtaccctgcaaggtctggtgtcctggggaactttcccttgcggccaacccaatgacccaggagtctacactcaagtgtgcaagttcaccaagtggataaatgacaccatgaaaaagcatcgctaacgccacactgagttaattaactgtgtgcttccaacagaaaatgcacaggagtgaggacgccgatgacctatgaagtcaaatttgactttacctttcctcaaagatatatttaaacctcatgccctgttgataaaccaatcaaattggtaaagacctaaaaccaaaacaaataaagaaacacaaaaccctcagtgctggagaagagtcagtgagaccagcactctcaaacactggaactggacgttcgtacagtctttacggaagacacttggtcaacgtacaccgagacccttattcaccacctttgacccagtaactctaatcttaggaagaacctactgaaacaaaaaaaatccaaaatgtagaacaagacttgaatttaccatgatattatttatcacagaaatgaagtgaaaccatcaaacatgttccaaaagtaccagatggcttaaataatagtctggcttggcacaacgatgttttttttctttgagacagagtctctgttgcttgggctgcaatgcagtgatgcaatcttggctcactgcaacctccgcctcctgggttcaagtgattctcgtgcttcagcctcccaagtacctgggactacaggtgtgcaccaccacaccaggctaattttttgtgtatttttactagagacagggtttcaccatgttggccagcgtggtcttgaacgcctgacctcagatgatccacccaccttggcctcccaaagtgctggga

SEQ ID NO: 13 - DNA, 1055 bp, Human
  5′UTR (1)..(102)
  CDS (103)..(861)
  3′UTR (862)..(1055)
tgccagcccaagtcggaacttggatcacatcagatcctctcgagctccagcaggagaggcccttcctcgcctggcagcccctgagcggctcagcagggcaccatggcaagatcccttctcctgcccctgcagatcctactgctatccttagccttggaaactgcaggagaagaagcccagggtgacaagattattgatggcgccccatgtgcaagaggctcccacccatggcaggtggccctgctcagtggcaatcagctccactgcggaggcgtcctggtcaatgagcgctgggtgctcactgccgcccactgcaagatgaatgagtacaccgtgcacctgggcagtgatacgctgggcgacaggagagctcagaggatcaaggcctcgaagtcattccgccaccccggctactccacacagacccatgttaatgacctcatgctcgtgaagctcaatagccaggccaggctgtcatccatggtgaagaaagtcaggctgccctcccgctgcgaaccccctggaaccacctgtactgtctccggctggggcactaccacgagcccagatgtgacctttccctctgacctcatgtgcgtggatgtcaagctcatctccccccaggactgcacgaaggtttacaaggacttactggaaaattccatgctgtgcgctggcatccccgactccaagaaaaacgcctgcaatggtgactcagggggaccgttggtgtgcagaggtaccctgcaaggtctggtgtcctggggaactttcccttgcggccaacccaatgacccaggagtctacactcaagtgtgcaagttcaccaagtggataaatgacaccatgaaaaagcatcgctaacgccacactgagttaattaactgtgtgcttccaacagaaaatgcacaggagtgaggacgccgatgacctatgaagtcaaatttgactttacctttcctcaaagatatatttaaacctcatgccctgttgataaaccaatcaaattggtaaagacctaaaaccaaaacaaataaagaaacacaaaaccctca

SEQ ID NO: 14 - protein, 253 aa, Human; identical to SEQ ID NO: 11

SEQ ID NO: 15 - DNA, 1923 bp, Human
  5′UTR (1)..(102)
  (CDS) (103)..(861)
  3′UTR (862)..(1923)
tgccagcccaagtcggaacttggatcacatcagatcctctcgagctccagcaggagaggcccttcctcgcctggcagcccctgagcggctcagcagggcaccatggcaagatcccttctcctgcccctgcagatcctactgctatccttagccttggaaactgcaggagaagaagcccagggtgacaagattattgatggcgccccatgtgcaagaggctcccacccatggcaggtggccctgctcagtggcaatcagctccactgcggaggcgtcctggtcaatgagcgctgggtgctcactgccgcccactgcaagatgaatgagtacaccgtgcacctgggcagtgatacgctgggcgacaggagagctcagaggatcaaggcctcgaagtcattccgccaccccggctactccacacagacccatgttaatgacctcatgctcgtgaagctcaatagccaggccaggctgtcatccatggtgaagaaagtcaggctgccctcccgctgcgaaccccctggaaccacctgtactgtctccggctggggcactaccacgagcccagatgtgacctttccctctgacctcatgtgcgtggatgtcaagctcatctccccccaggactgcacgaaggtttacaaggacttactggaaaattccatgctgtgcgctggcatccccgactccaagaaaaacgcctgcaatggtgactcagggggaccgttggtgtgcagaggtaccctgcaaggtctggtgtcctggggaactttcccttgcggccaacccaatgacccaggagtctacactcaagtgtgcaagttcaccaagtggataaatgacaccatgaaaaagcatcgctaacgccacactgagttaattaactgtgtgcttccaacagaaaatgcacaggagtgaggacgccgatgacctatgaagtcaaatttgactttacctttcctcaaagatatatttaaacctcatgccctgttgataaaccaatcaaattggtaaagacctaaaaccaaaacaaataaagaaacacaaaaccctcagtgctggagaagagtcagtgagaccagcactctcaaacactggaactggacgttcgtacagtctttacggaagacacttggtcaacgtacaccgagacccttattcaccacctttgacccagtaactctaatcttaggaagaacctactgaaacaaaaaaaatccaaaatgtagaacaagacttgaatttaccatgatattatttatcacagaaatgaagtgaaaccatcaaacatgttccaaaagtaccagatggcttaaataatagtctggcttggcacaacgatgttttttttctttgagacagagtctctgttgcttgggctgcaatgcagtgatgcaatcttggctcactgcaacctccgcctcctgggttcaagtgattctcgtgcttcagcctcccaagtacctgggactacaggtgtgcaccaccacaccaggctaattttttgtgtatttttactagagacagggtttcaccatgttggccagcgtggtcttgaacgcctgacctcagatgatccacccaccttggcctcccaaagtgctgggattacaggcatgagccaccacggccagcccacaatgatattacaaacctattaaaaatgatacttagacagaattgtcagtattattcaagaacatttaggctataggatgttaaatgacaaaaggaaggacaaaaatatatatgtatgtgaccctacccataaaaaatgaaatattcacagaatcagatctgaaaacacatgtcccagactgcatactggggtcgtcatgaggtgtctccttccttctgtgtacttttccttgaatgtgcacttttataacatgaaaaataaaggtggggaaaaaagtctgaaga

SEQ ID NO: 16 - DNA, 1756 bp, Human
  mRNA (1)..(188): Exon 1
agataaccagaaccgcaaatgcaggcgggggtgtcccagagtcggctccgcctgcaccccagggcgctggggccgggcatggggcggggggtgatataagaggacggcccagcagagggctgaagattttggagcccagctgtgtgccagcccaagtcggaacttggatcacatcagatcctctcgagctccagcaggagaggcccttcctcgcctggcagcccctgagcggctcagcagggcaccatggcaagatcccttctcctgcccctgcagatcttactgctatccttagccttggaaactgcaggagaagaagcccagggtgacaagattattgatggcgccccatgtgcaagaggctcccacccatggcaggtggccctgctcagtggcaatcagctccactgcggaggcgtcctggtcaatgagcgctgggtgctcactgccgcccactgcaagatgaatgagtacaccgtgcacctgggcagtgatacgctgggcgacaggagagctcagaggatcaaggcctcgaagtcattccgccaccccggctactccacacagacccatgttaatgacctcatgctcgtgaagctcaatagccaggccaggctgtcatccatggtgaagaaagtcaggctgccctcccgctgcgaaccccctggaaccacctgtactgtctccggctggggcactaccacgagcccagatgtgacctttccctctgacctcatgtgcgtggatgtcaagctcatctccccccaggactgcacgaaggtttacaaggacttactggaaaattccatgctgtgcgctggcatccccgactccaagaaaaacgcctgcaatggtgactcagggggaccgttggtgtgcagaggtaccctgcaaggtctggtgtcctggggaactttcccttgcggccaacccaatgacccaggagtctacactcaagtgtgcaagttcaccaagtggataaatgacaccatgaaaaagcatcgctaacgccacactgagttaattaactgtgtgcttccaacagaaaatgcacaggagtgaggacgccgatgacctatgaagtcaaatttgactttacctttcctcaaagatatatttaaaccaacctcatgccctgttgataaaccaatcaaattggtaaagacctaaaaccaaaacaaataaagaaacacaaaaccctcagtgctggagaagagtcagtgagaccagcactctcaaacactggaactggacgttcgtacagtctttacggaagacacttggtcaacgtacaccgagacccttattcaccacctttgacccagtaactctaatcttaggaagaacctactgaaacaaaaaaaatccaaaatgtagaacaagacttgaatttaccatgatattatttatcacagaaatgaagtgaaaccatcaaacatgttccaaaagtaccagatggcttaaataatagtctggcttggcacaacgatgttttttttctttgagacagagtctctgttgcttgggctgcaatgcagtgatgcaatcttggctcactgcaacctccgcctcctgggttcaagtgattctcgtgcttcagcctcccaagtacctgggactacaggtgtgcaccaccacaccaggctaattttttgtgtatttttactagagacagggtttcaccatgttggccagcatggtcttgaacgcctgacctcagatgatccacccaccttggcctcccaaagtgctggga

SEQ ID NO: 17 - protein, 253 aa, Human; identical to SEQ ID NO: 11

SEQ ID NO: 18 - DNA, 1054 bp, Human
  mRNA (1)..(188): Exon 1
agataaccagaaccgcaaatgcaggcgggggtgtcccagagtcggctccgcctgcaccccagggcgctggggccgggcatggggcggggggtgatataagaggacggcccagcagagggctgaagattttggagcccagctgtgtgccagcccaagtcggaacttggatcacatcagatcctctcgagcccagggtgacaagattattgatggcgccccatgtgcaagaggctcccacccatggcaggtggccctgctcagtggcaatcagctccactgcggaggcgtcctggtcaatgagcgctgggtgctcactgccgcccactgcaagatgaatgagtacaccgtgcacctgggcagtgatacgctgggcgacaggagagctcagaggatcaaggcctcgaagtcattccgccaccccggctactccacacagacccatgttaatgacctcatgctcgtgaagctcaatagccaggccaggctgtcatccatggtgaagaaagtcaggctgccctcccgctgcgaaccccctggaaccacctgtactgtctccggctggggcactaccacgagcccagatgtgacctttccctctgacctcatgtgcgtggatgtcaagctcatctccccccaggactgcacgaaggtttacaaggacttactggaaaattccatgctgtgcgctggcatccccgactccaagaaaaacgcctgcaatggtgactcagggggaccgttggtgtgcagaggtaccctgcaaggtctggtgtcctggggaactttcccttgcggccaacccaatgacccaggagtctacactcaagtgtgcaagttcaccaagtggataaatgacaccatgaaaaagcatcgctaacgccacactgagttaattaactgtgtgcttccaacagaaaatgcacaggagtgaggacgccgatgacctatgaagtcaaatttgactttacctttcctcaaagatatatttaaaccaacctcatgccctgttgataaaccaatcaaattggtaaagacctaaaaccaaaacaaataa

SEQ ID NO: 19 - protein, 181 aa, Human
MNEYTVHLGSDTLGDRRAQRIKASKSFRHPGYSTQTHVNDLMLVKLNSQARLSSMVKKVRLPSRCEPPGTTCTVSGWGTTTSPDVTFPSDLMCVDVKLISPQDCTKVYKDLLENSMLCAGIPDSKKNACNGDSGGPLVCRGTLQGLVSWGTFPCGQPNDPGVYTQVCKFTKWINDTMKKHR

SEQ ID NO: 20 - DNA, 131 bp, Human
ctccagcaggagaggcccttcctcgcctggcagcccctgagcggctcagcagggcaccatggcaagatcccttctcctgcccctgcagatcttactgctatccttagccttggaaactgcaggagaagaag

SEQ ID NO: 21 - protein, 48 aa, Human
AQGDKIIDGAPCARGSHPWQVALLSGNQLHCGGVLVNERWVLTAAHCK

SEQ ID NO: 22 - DNA, 30 bp, Artificial Sequence
  18S oligonucleotide probe
cggcatgtattagctctagaattaccacag

SEQ ID NO: 23 - DNA, 21 bp, Artificial Sequence
  beta 2-microglobulin PCR primer 1
tgaattgctatgtgtctgggt

SEQ ID NO: 24 - DNA, 21 bp, Artificial Sequence
  beta 2-microglobulin PCR primer 2
cctccatgatgctgcttacat

SEQ ID NO: 25 - DNA, 23 bp, Artificial Sequence
  Internal KLK5 primer
agtgcacttgggggctcttggtt

SEQ ID NO: 26 - DNA, 21 bp, Artificial Sequence
  Internal KLK7 primer
gccgaggtgcacggtgtactc

SEQ ID NO: 27 - protein, 17 aa, Artificial Sequence
  hK5 mid region peptide
RIRPTKDVRPINVSSHC

SEQ ID NO: 28 - protein, 14 aa, Artificial Sequence
  hK5 C-terminal peptide
CEDAYPRQIDDTMF

SEQ ID NO: 29 - DNA, 20 bp, Artificial Sequence
  K5Ex3S
gagctggggccggggaagac

SEQ ID NO: 30 - DNA, 20 bp, Artificial Sequence
  K5Ex6AS
tgggccgggcacaagggtaa

SEQ ID NO: 31 - DNA, 20 bp, Artificial Sequence
  K5StartS
tcaggcccggtgcccagagc

SEQ ID NO: 32 - DNA, 20 bp, Artificial Sequence
  K5Ex3AS
tcatccgaccgggcgtcttc

SEQ ID NO: 33 - DNA, 21 bp, Artificial Sequence
  K5Ex4AS
gaatctgggcagcagatgttc

SEQ ID NO: 34 - DNA, 21 bp, Artificial Sequence
  K7Ex3S
gcccagggtgacaagattatt

SEQ ID NO: 35 - DNA, 20 bp, Artificial Sequence
  K7Ex6AS
gtacctctgcacaccaacgg

SEQ ID NO: 36 - DNA, 26 bp, Artificial Sequence
  K7StartS
agataaccagaaccgcaaatgcaggc

SEQ ID NO: 37 - DNA, 21 bp, Artificial Sequence
  K7Ex4AS
catgggtctgtgtggagtagc

SEQ ID NO: 38 - DNA, 21 bp, Artificial Sequence
  K75′UTR
ggatcacatcagatcctctcg

SEQ ID NO: 39 - DNA, 21 bp, Artificial Sequence
  K73′UTshAS
tcataggtcatcggcgtcctc

SEQ ID NO: 40 - DNA, 21 bp, Artificial Sequence
  K7Ex2S
cccctgcagatcttactgcta

SEQ ID NO: 41 - DNA, 20 bp, Artificial Sequence
  K73′UTRAS
ggcgttcaagaccatgctgg

(19) United States
(12) Patent Application Publication
(10) Pub. No.: US 2006/0035219 A1
(43) Pub. Date: Feb. 16, 2006

(54) Aberrant kallikrein expression

(76) Inventor: Judith Ann Clements, Qucensland (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/412,748

(22) Filed: Apr. 9, 2003

(30) Foreign Application Priority Data
Apr. 9, 2002 (AU) PS1616/02

(51) Int. Cl. C12Q 1/68; G01N 33/574; C07H 21/04; C12P 21/06; C12N 9/64

(52) U.S. Cl. 435/6; 435/7.23; 435/69.1; 435/226; 435/320.1; 435/325; 536/23.2

(57) ABSTRACT

The present invention relates to differentially expressed kallikrein molecules and more particularly to novel expression products of the KLK5 and KLK7 genes, which are differentially expressed between normal and cancer cells and which serve as targets for diagnosis and therapeutic intervention of cancers, especially serous cancers such as ovarian cancer.

### FIELD OF THE INVENTION

THIS INVENTION relates generally to differentially expressed molecules. More particularly, the present invention relates to novel expression products of the KLK5 and KLK7 genes, which are differentially expressed between normal and cancer cells. Even more particularly, the present invention relates to isolated polynucleotides comprising a nucleotide sequence that corresponds or is complementary to at least a portion of an aberrant KLK5 polynucleotide that correlates with the presence or risk of a cancer or related condition. The invention also encompasses isolated polynucleotides comprising a nucleotide sequence that corresponds or is complementary to at least a portion of a KLK7 polynucleotide that is aberrantly expressed in a cancer or related condition as well as to isolated polypeptides comprising an amino acid sequence encoded by the at least a portion of a KLK7 polynucleotide that is so aberrantly expressed. The invention also extends to variants and derivatives of these molecules, to vectors comprising the aforesaid isolated polynucleotides and to host cells containing such vectors. The invention further extends to antigen-binding molecules that are immuno-interactive with a K7 polypeptide that is aberrantly expressed in a cancer or related condition and to the use of these antigen-binding molecules as well as the aforementioned isolated polynucleotides and polypeptides in assays and kits for detecting the presence or diagnosing the risk of a cancer or related condition. The present invention also relates to a method for detecting the presence or diagnosing the risk of a cancer or related condition, either before or after the onset of clinical symptoms, by detecting expression of the aberrant KLK5 polynucleotide or aberrant expression of the KLK7 polynucleotide. The invention further encompasses the use of agents that modulate the level and/or functional activity of an aberrant KLK5 expression product or an aberrantly expressible KLK7 expression product for treating and/or preventing the cancer or related condition.

Bibliographic details of the publications numerically referred to in this specification are collected at the end of the description.

### BACKGROUND OF THE INVENTION

Ovarian carcinoma is the second most common and the leading cause of death from gynecologic malignancy (1). The overall 5-year survival rate of ovarian cancer patients is less than 50%, because most of these patients are diagnosed at an advanced stage of the disease, in which the primary tumor has progressed to a highly invasive and metastatic state (2). Thus, development of a reliable early diagnostic system and appreciate treatment is very important in the management and treatment of ovarian carcinomas.

The kallikreins (KLKs) are a subgroup of serine proteases that have been recently expanded to 15 members and localized to chromosome 19q13.4 (3, 4). Many of these genes are highly expressed in ovarian cancer. The present inventors have previously shown high expression of KLK4 (prostase, KLK-L1, EMSP-1) and its mRNA variants, as well as the hK4 protein in ovarian epithelial carcinoma (5); others have shown that higher KLK4 mRNA levels in ovarian cancer tissues are related to poor prognosis (6). Aberrant expression of KLK6/hK6 (alternatively named protease M, zyme, neurosin) (7-9), KLK8/hK8 (neuropsin, ovasin, tumor-associated differentially expressed gene-14, TADG-14) (10, 11), KLK9 (12) and KLK10 (normal epithelial-specific 1 gene, NES1) (13) were also found in ovarian carcinomas. KLK5 (alternatively named stratum corneum tryptic enzyme, SCTE) (14) and KLK7 (stratum corneum chymotryptic enzyme, SCCE) (15, 16) were originally identified from a keratinocyte library, and their enzymes were purified from stratum corneum of human skin. KLK7 catalyses the degradation of intercellular cohesive structures in the outermost layer of the skin and contributes to the cell shedding process at the skin surface (16). In addition, these two enzymes showed co-expression in skin tissue and these authors suggested that hK5 may be the physiological activator of hK7 (17). Recently three different groups independently reported that KLK5 (18, 19) and KLK7/hK7 (20) were highly expressed in ovarian cancers, respectively. However, expression of the hK5 protein in ovarian tumors has not yet been documented, and the relationship between the expression patterns of KLK5 and KLK7 in this tumor is not known.

### SUMMARY OF THE INVENTION

The present invention is predicated in part on the identification of novel KLK5 and KLK7 variant mRNA transcripts from ovarian cancer cell lines and/or normal ovarian epithelial cells. The present inventors have also found that these transcripts, and in certain instances, their polypeptide products, are differentially expressed between cancer cells (e.g., prostate or ovarian cancer cells) and normal cells. Based on pathophysiologies associated with other members of the KLK gene family, it is proposed that such aberrant expression of KLK5 and KLK7 may potentially relate to other hormone-associated carcinomas, especially serous carcinomas. The foregoing discoveries have been reduced to practice in methods of diagnosing various conditions associated with aberrant expression of KLK5 and KLK7, in new isolated molecules for use in such diagnosis, and in compositions for treating and/or preventing the aforesaid conditions.

Accordingly, in one aspect of the present invention, there is provided a method for detecting the presence or diagnosing the risk of a cancer or related condition in a patient, comprising detecting the presence of an aberrant KLK5 expression product or the aberrant expression of a KLK7 expression product in a biological sample obtained from the patient, wherein the aberrant expression product or the aberrant expression correlates with the presence or risk of the cancer or related condition. In one embodiment, the cancer is regulatable by a hormone including, but not restricted to, testosterone, estrogen and progesterone. In another embodiment, the cancer is a serous cancer, which is preferably a serous carcinoma such as ovarian cancer.

The aberrant KLK5 expression product is preferably an aberrant KLK5 polynucleotide, which suitably comprises a substitution, deletion and/or addition of one or more nucleotides relative to normal KLK5. In one embodiment of this type, the aberrant KLK5 polynucleotide comprises a deletion corresponding to all or part of exon 1 of normal KLK5 as set forth in SEQ ID NO: 4. Preferably, the deletion comprises all or part of the sequence set forth in SEQ ID NO: 5. In an especially preferred embodiment of this type, the aberrant KLK5 polynucleotide comprises the sequence set forth in SEQ ID NO: 9.

Aberrant expression of a KLK7 expression product includes and encompasses a level and/or functional activity of that expression product, which differs from a normal reference level and/or functional activity. In a preferred embodiment, the aberrantly expressible KLK7 expression product is expressed at a higher level and/or functional activity than the normal reference level and/or functional activity. The aberrantly expressible KLK7 expression product preferably comprises all or part of exon 2 as set forth in SEQ ID NO: 20. In a preferred embodiment of this type, the aberrantly expressible KLK7 polynucleotide comprises the sequence set forth in SEQ ID NO: 16. In another preferred embodiment of this type, the aberrantly expressible KLK7 expression product is suitably an aberrantly expressible K7 polypeptide comprising all or part of the amino acid sequence set forth in SEQ ID NO: 21. More preferably, the aberrantly expressible K7 polypeptide comprises the sequence set forth in SEQ ID NO: 17.

In another aspect, the invention encompasses the use of at least a portion of an aberrant KLK5 expression product, or of at least a portion of a KLK7 expression product, as broadly described above, or of one or more antigen-binding molecules that are immuno-interactive specifically with an aberrant K7 polypeptide as broadly described above, in the manufacture of a kit for detecting an aberrant KLK5 expression product or an aberrant expressed KLK7 expression product, which correlates with the presence or risk of a cancer or related condition.

In yet another aspect, the invention contemplates the use of an agent in the manufacture of a medicament for modulating the level and/or functional activity of an aberrant KLK5 expression product or of an aberrantly expressible KLK7 expression product as broadly described above in a patient, wherein the agent is optionally formulated with a pharmaceutically acceptable carrier and is identifiable by a screening assay comprising:
contacting a preparation comprising at least a portion of an aberrant KLK5 expression product or at least a portion of an aberrantly expressible KLK7 expression product, or a variant or derivative of these, with the agent; and
detecting a change in the level and/or functional activity of the at least a portion of the expression product(s) or the variant or derivative.

In one embodiment of this type, the agent is an antisense oligonucleotide or ribozyme that binds to, or otherwise interacts specifically with, an aberrant KLK5 transcript or an aberrantly expressible KLK7 expression product. In another embodiment of this type, the agent is selected from one or more antigen-binding molecules that are immuno-interactive with an aberrantly expressible K7 polypeptide.

In still yet another aspect, the invention contemplates the use of an agent in the manufacture of a medicament for treating and/or preventing a cancer or related condition, wherein the agent is optionally formulated with a pharmaceutically acceptable carrier and is identifiable by a screening assay comprising:

contacting a preparation comprising at least a portion of an aberrant KLK5 expression product or at least a portion of an aberrantly expressible KLK7 expression product, or a variant or derivative of these, with the agent; and

detecting a change in the level and/or functional activity of the at least a portion of the expression product(s) or the variant or derivative.

In still yet another aspect, the invention features a method for modulating the level of an aberrantly expressible KLK5 expression product or of an aberrantly expressible KLK7 expression product in a patient in need of such treatment, comprising administering to the patient an effective amount of an agent as broadly described above and optionally a pharmaceutically acceptable carrier.

According to another aspect, the invention provides a method for the treatment and/or prophylaxis of a cancer or related condition, comprising administering to a patient in need of such treatment an effective amount of an agent as broadly described above and optionally a pharmaceutically acceptable carrier.

In yet another aspect, the invention contemplates an isolated polynucleotide comprising a nucleotide sequence which corresponds or is complementary to at least a portion of an aberrant KLK5 polynucleotide as broadly described above that correlates with the presence or risk of a cancer or related condition.

In still another aspect, the invention provides an isolated polynucleotide comprising a nucleotide sequence which corresponds or is complementary to at least a portion of an aberrantly expressible KLK7 polynucleotide as broadly described above that correlates with the presence or risk of a cancer or related condition.

The at least a portion of the aberrant KLK5 polynucleotide or of the aberrantly expressible KLK7 polynucleotide suitably comprises at least 10, preferably at least 15, more preferably at least 18 and even more preferably at least 20 nucleotides.

The present invention also encompasses a method of identifying aberrant expression products, which correlate with the presence or risk of a cancer or related condition, comprising determining the sequence of a KLK5 expression product from subjects known to have the cancer or related condition and comparing the sequence to that of wild-type KLK5 expression products to thereby identify the aberrant expression products.

According to another aspect, the invention provides a method of identifying KLK7 expression products, which are aberrantly expressible and which correlate with the presence or risk of a cancer or related condition, comprising comparing the level and or functional activity of a KLK7 expression product which is present in a biological sample obtained from a subject known to have the cancer or related condition to that which is present in a corresponding biological sample obtained from a normal subject or from a subject who is not afflicted with the cancer or related condition, to thereby identify the aberrantly expressible KLK7 expression products.

The invention in yet another aspect contemplates a probe for interrogating nucleic acid for the presence of nucleic acid molecules that are associated with a cancer or a related condition, comprising a nucleotide sequence which corresponds or is complementary to a portion of an aberrant KLK5 polynucleotide or an aberrantly expressible KLK7 polynucleotide as broadly described above that correlates with the presence or risk of the cancer or related condition.

In yet another aspect, the invention provides a vector comprising an isolated polynucleotide as broadly described above, or a probe as broadly described above.

In still another aspect, the invention encompasses an expression vector comprising an isolated polynucleotide as broadly described above, operably linked to a regulatory polynucleotide.

In another aspect, the invention provides a host cell containing a vector or expression vector as broadly described above.

In yet another aspect, the invention provides a cell line comprising at least a portion of an aberrant KLK5 polynucleotide or an aberrantly expressible KLK7 polynucleotide as broadly described above. Preferably, the cell line is derived from a patient who is afflicted with the cancer or related condition.

In another aspect, the invention encompasses an isolated polypeptide comprising an amino acid sequence which corresponds to at least a portion of an aberrant K7 polypeptide as broadly described above that correlates with the presence or risk of a cancer or related condition. The at least a portion of the aberrant K7 polypeptide preferably comprises at least 5, preferably at least 10, more preferably at least 20 and even more preferably at least 50 amino acids.

In a further aspect, the invention provides one or more antigen-binding molecules that are immuno-interactive with, and provide specificity for detection of, an aberrantly expressible K7 polypeptide as broadly described above.

The invention also encompasses the use of an isolated polynucleotide as broadly described above, the use of a probe as broadly described above, the use of an isolated polypeptide as broadly described above or the use of an antigen-binding molecule as broadly described above for detecting an aberrant KLK5 expression product, or an aberrantly expressible KLK7 expression product as broadly described above that correlate with a cancer or related condition.

### BRIEF DESCRIPTION OF THE DRAWINGS

FIG. 1 is a schematic representation of human KLK5 (A) and KLK7 (B) cDNA and the positions of primers used to detect KLK5 and KLK7 expression respectively. The numbered exons indicate primarily coding exons except for exon 1 which has only 5′UTR sequences. The sequences of the primers are denoted 5′ to 3′.

FIG. 2 is a photographic and graphical representation showing KLK5 and KLK7 expression in normal ovaries and ovarian tumors. A and B: Analysis of amplification of KLK5 (A) and KLK7 (B) in ovarian epithelial cells for different PCR cycles. Lanes 1-2, 25 cycles; Lanes 3-4, 30 cycles; Lanes 5-6, 35 cycles; Lanes 7-8, 40 cycles; Lanes 1, 3, 5 and 7, NOE; Lanes 2, 4, 6 and 8 SER carcinoma cells; and Lane 9, negative control (no cDNA). C and E: Southern blot analysis of the KLK5 and KLK7 RT-PCR products respectively, with DIG-labeled internal KLK5 and KLK7 probes probe. D and F: Densitometric analysis of the Southern blots C and E, respectively. G. Ethidium bromide stained agarose gel of the RT-PCR for β2-microglobulin as an internal control. Lanes 1-3, normal ovarian tissues; Lanes 4-6, normal ovarian epithelial cells (NOE); Lanes 7-8, primary cultured cells from serous adenomas of ovary (BNG: benign); Lanes 9-10, primary cultured cells from stage II serous carcinomas; Lanes 11-14, primary cultured cells from stage III and IV serous carcinomas of ovary; Lanes 15-16, mucinous carcinoma tissues (MUC); Lanes 17-18, primary cultured cells from endometrioid carcinomas (END); Lanes 19-20, primary cultured cells from clear cell carcinomas (CCC); Lanes 21-22, granulosa cell tumor tissues (GCT); Lanes 23-25, serous ovarian carcinoma cell line OVCAR-3, keratinocyte line HaCat and negative control, respectively.

FIG. 3 is a photographic representation showing a Northern blot analysis of mRNA hybridized with the ³²P-labeled cRNA KLK5 probe (A), KLK7 probe (B) and 18S control from normal ovary and ovarian carcinomas. Lane 1, Normal ovarian epithelial cells (NOE); Lane 2, Serous ovarian carcinoma cells (Ser Ca); Lane 3, ovarian carcinoma cell line OVCAR-3; and Lane 4, HaCat cells.

FIG. 4 is a photographic and schematic representation showing expression of KLK5 (A and B) and KLK7 (C-H) transcripts in normal and malignant ovarian epithelial cells. A. Expression of KLK5 5′UTR region by RT-PCR in NOE and OVCAR-3 cells with primers K5StartS and K5E×3AS indicating the 494 and 290 bp products. B. Schematic diagrams showing the first 3 exons of KLK5 and the different 5′UTR regions in ovarian epithelial cells. Boxed numbers indicate size in bp of exons; slash indicates ATG coding start site in exon 2. The RT-PCR product sizes are indicated below. C and D. Schematic representation of the long KLK7 (C) and short KLK7 (D) transcripts. Boxed numbers indicate size in bp of exons; slash indicates ATG coding start site in exon 2 or exon 3. The 5′UTR, 3′UTR and coding regions are indicated. E. Amino acid sequence alignment of long and short forms of hK7. The five exons of the coding region are marked, and the introns are indicated by a dotted line ( . . . ). The amino acids that constitute the catalytic triad, Histidine (H), Aspartic (D), and Serine (S) are marked in bold. * indicates the end of the protein sequence. F. Ethidium bromide stained agarose gel of the RT-PCR using primers K7 Starts and K7E×4AS showing long KLK7 (573 bp) and short KLK7 (exon 2 deleted) (442 bp) in NOE and PEO1 cells. G. RT-PCR with K7 5′UTR and K7 3′UtshAS primers showing expression of long (915 bp) and short (784 bp) KLK7 transcripts in NOE and PEO1. H. Expression of KLK7 long transcript (1308 bp) in NOE and PEO1 by RT-PCR with K7E×2S and K7 3′UTRAS primers.

FIG. 5 is a photographic representation showing expression of hK5 and hK7 in NOE, ovarian cancer cell lines and ovarian tumor cells by Western blot analysis. (A) Western blot with anti-hK5 antibodies. Lanes 1-4, Western blot with anti-hK5 pro-form region antibody; Lanes 5-12, Western blot with anti-hK5 peptide antibody to the active form. Lane 1, Normal ovarian epithelial cells (NOE); Lane 2, Benign serous adenoma (BNG); Lane 3, serous ovarian carcinoma cells; Lane 4, Ovarian cancer cell line PEO1; Lane 5, NOE; Lane 6, BNG; Lanes 7-8, Serous ovarian carcinoma cells; Lanes 9-10, ovarian cancer cell lines OVCAR-3 and PEO1; Lane 11, PEO1 conditioned media; and Lane 12, Keratinocyte cell line HaCat. (B) Western blot with anti-hK7 antibody. Lane 1, NOE; Lane 2, BNG; Lane 3-4, Serous ovarian carcinoma cells; Lane 5 and 6, ovarian cancer cell lines OVCAR-3 and PEO1; Lane 7, PEO1 conditioned media; and Lane 8, HaCat. Re-incubation of the Western blots with a Tubulin antibody shows the protein loading.

FIG. 6 is a photographic representation showing representative expression of hK5 and hK7 in ovarian tumor biopsies. (A-D) hK5 expression as detected with the anti-hK5 pro-form region antibody. (A) Benign serous cystadenoma of ovary showing weak hK5 expression. (B) and (C) Moderately differentiated serous ovarian carcinomas showing cytoplasmic expression of hK5 (solid arrows). (E-H) hK5 expression as detected with anti-hK5 peptide antibody (active form region). (E) BNG serous cystadenoma of ovary showing weak hK5 expression. (F) and (G) Moderately differentiated serous ovarian carcinomas showing apical membrane and cytoplasmic expression of hK5 (solid arrows). (I-L) hK7 expression as detected with anti-recombinant hK7 antibody. (I) BNG serous cystadenoma showing weak hK7 expression. (J) Moderately differentially serous ovarian carcinoma showing apical membrane and cytoplasmic expression (solid arrow), and the invading cancer cells showing cytoplasmic (arrow head) expression. (K) Moderately differentiated serous ovarian carcinomas showing apical membrane (open arrow) and cytoplasmic (solid arrow) expression. No staining shown by stroma cells (open arrows). (D), (H) and (L) Negative control performed with 10% normal goat serum instead of primary antibodies. Magnification: A, E and I, × 120; B, C, F, G, J and K, × 60; D and H, ×80; L ×100.

### BRIEF DESCRIPTION OF THE SEQUENCES: SUMMARY TABLE

TABLE A

| SEQUENCE ID | | | |
|---|---|---|---|
| NUMBER | SEQUENCE | LENGTH | |
| SEQ ID NO: 1 | Nucleotide sequence corresponding to the human KLK5 gene | 9367 | nts |
| | as set forth in GenBank Accession No. 18602112 | | |
| SEQ ID NO: 2 | K5 polypeptide encoded by SEQ ID NO: 1 | 293 | aa |
| SEQ ID NO: 3 | Nucleotide sequence corresponding to a revised human KLK5 | 9755 | nts |
| | gene, comprising an additional exon upstream of exon 1 | | |
| | defined in SEQ ID NO: 1 | | |
| SEQ ID NO: 4 | Nucleotide sequence corresponding to the novel upstream | 314 | nts |
| | exon of SEQ ID NO: 3 | | |
| SEQ ID NO: 5 | Nucleotide sequence corresponding to a portion of the novel | 242 | nts |
| | upstream exon of SEQ ID NO: 3, which is deleted in an | | |
| | alternately spliced variant of human KLK5 as set forth in | | |
| | SEQ ID NO: 9 | | |
| SEQ ID NO: 6 | Nucleotide sequence corresponding to normal KLK5 mRNA | 1222 | nts |
| | as set forth in GenBank Accession No. XM_055656 | | |
| SEQ ID NO: 7 | K5 polypeptide encoded by SEQ ID NO: 6 | 293 | aa |
| SEQ ID NO: 8 | Nucleotide sequence corresponding to an alternately spliced | 1532 | nts |
| | variant of human KLK5 (long form) | | |
| SEQ ID NO: 9 | Nucleotide sequence corresponding to another alternately | 1329 | nts |
| | spliced variant of human KLK5 (short form) | | |
| SEQ ID NO: 10 | Nucleotide sequence corresponding to the human KLK7 gene | 9729 | nts |
| | as set forth in GenBank Accession No. AF332583 | | |
| SEQ ID NO: 11 | K7 polypeptide encoded by SEQ ID NO: 10 | 253 | aa |
| SEQ ID NO: 12 | Nucleotide sequence corresponding to a revised human KLK7 | 8831 | nts |
| | gene, comprising an additional upstream sequence for exon 1 | | |
| | and a shorter exon 6 relative to the sequence presented in | | |
| | SEQ ID NO: 10 | | |
| SEQ ID NO: 13 | Nucleotide sequence corresponding to a first KLK5 transcript | 1055 | nts |
| | as set forth in GenBank Accession No. AF332583 | | |
| SEQ ID NO: 14 | K7 polypeptide encoded by SEQ ID NO: 13 | 253 | aa |
| SEQ ID NO: 15 | Nucleotide sequence corresponding to a second KLK5 | 1923 | nts |
| | transcript as set forth in GenBank Accession No. AF332583 | | |
| SEQ ID NO: 16 | Nucleotide sequence corresponding to an alternately spliced | 1756 | nts |
| | variant of human KLK7 (long form) | | |
| SEQ ID NO: 17 | Aberrantly expressible K7 polypeptide encoded by SEQ ID | 253 | aa |
| | NO: 16 | | |
| SEQ ID NO: 18 | Nucleotide sequence corresponding to another alternately | 1054 | nts |
| | spliced variant of human KLK7 (short form) | | |
| SEQ ID NO: 19 | K7 polypeptide variant encoded by SEQ ID NO: 18 | 181 | aa |
| SEQ ID NO: 20 | Nucleotide sequence corresponding to exon 2 which is | 131 | nts |
| | deleted in an alternately spliced variant of human KLK7 as | | |
| | set forth in SEQ ID NO: 18 | | |
| SEQ ID NO: 21 | Amino acid sequence deleted in the short form of human K7 | 48 | aa |
| | set forth in SEQ ID NO: 19 | | |
| SEQ ID NO: 22 | 18S oligonucleotide probe | 30 | nts |
| SEQ ID NO: 23 | β2-microglobulin PCR primer 1 | 21 | nts |
| SEQ ID NO: 24 | β2-microglobulin PCR primer 2 | 21 | nts |
| SEQ ID NO: 25 | Internal KLK5 primer | 23 | nts |
| SEQ ID NO: 26 | Internal KLK7 primer | 21 | nts |
| SEQ ID NO: 27 | hK5 mid region peptide | 17 | aa |
| SEQ ID NO: 28 | hK5 C-terminal peptide | 14 | aa |
| SEQ ID NO: 29 | K5Ex3S primer | 20 | nts |
| SEQ ID NO: 30 | K5Ex6AS primer | 20 | nts |
| SEQ ID NO: 31 | K5StartS primer | 20 | nts |
| SEQ ID NO: 32 | K5Ex3AS primer | 20 | nts |
| SEQ ID NO: 33 | K5Ex4AS primer | 21 | nts |
| SEQ ID NO: 34 | K7Ex3S primer | 21 | nts |
| SEQ ID NO: 35 | K7Ex6AS primer | 20 | nts |
| SEQ ID NO: 36 | K7StartS primer | 26 | nts |
| SEQ ID NO: 37 | K7Ex4AS primer | 21 | nts |
| SEQ ID NO: 38 | K75′UTR primer | 21 | nts |
| SEQ ID NO: 39 | K73′UtshAS primer | 21 | nts |
| SEQ ID NO: 40 | K7Ex2S primer | 21 | nts |
| SEQ ID NO: 41 | K73′UTRAS primer | 20 | nts |

### DETAILED DESCRIPTION OF THE INVENTION

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The term “aberrant polynucleotide” refers to a polynucleotide which differs from a “normal” reference polynucleotide by the substitution, deletion and/or addition of at least one nucleotide and which correlates with the presence or risk of a cancer or related condition.

The terms “aberrant polynucleotide variant” and “variant” refer to polynucleotides displaying substantial sequence identity with a reference aberrant polynucleotide sequence or polynucleotides that hybridize with a reference aberrant polynucleotide sequence under stringent conditions that are defined hereinafter, wherein the variant polynucleotides comprise the same alteration as the reference aberrant polynucleotide sequence, or an alteration that encodes the same aberrant amino acid(s) (silent alteration) encoded by the reference polynucleotide sequence. The terms “aberrant polynucleotide variant” and “variant” also include naturally occurring allelic variants.

The term “aberrant polypeptide” refers to a polypeptide which differs from a “normal” reference polypeptide by the substitution, deletion and/or addition of at least one amino acid residue and which correlates with the presence or risk of a cancer or related condition.

The term “aberrant polypeptide variant” refers to aberrant polypeptides which are distinguished from a normal polypeptide by the addition, deletion or substitution of at least one amino acid but otherwise comprise the same aberration which correlates with the presence or risk of a cancer or related condition. In this regard, it is well understood in the art that some amino acids may be changed to others with broadly similar properties without changing the nature of the activity of the aberrant polypeptide (conservative substitutions) as described hereinafter. Accordingly, the present invention encompasses immuno-mimetic polypeptides that can elicit the production of elements that are immuno-interactive with a naturally occurring aberrantly expressed K7 polypeptide.

“Amplification product” refers to a nucleic acid product generated by nucleic acid amplification techniques.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

“Antigenic or immunogenic activity” refers to the ability of a polypeptide, fragment, variant or derivative according to the invention to produce an antigenic or immunogenic response in a mammal to which it is administered, wherein the response includes the production of elements which specifically bind the polypeptide or fragment thereof.

The term “biological sample” as used herein refers to a sample that may be extracted, untreated, treated, diluted or concentrated from a patient. Suitably, the biological sample is selected from tissue samples including tissue from the ovaries, endometrium, and prostate. The biological sample may also be a fluid selected from the group consisting of whole blood, serum, plasma, saliva, urine, sweat, ascitic fluid, peritoneal fluid, synovial fluid, amniotic fluid, cerebrospinal fluid, skin biopsy, and the like. The biological sample preferably includes serum, whole blood, plasma, lymph and ovarian follicular fluid as well as other circulatory fluid and saliva, mucus secretion and respiratory fluid. More preferably, the biological sample is a circulatory fluid such as serum or whole blood or a fractionated portion thereof.

By “biologically active fragment” is meant a fragment of a full-length parent polypeptide which fragment retains the activity of the parent polypeptide. For example, a biologically active fragment of a K7 polypeptide will have the ability to elicit the production of elements that specifically bind to that polypeptide. As used herein, the term “biologically active fragment” includes deletion variants and small peptides, for example of at least 10, preferably at least 20 and more preferably at least 30 contiguous amino acids, which comprise the above activities. Peptides of this type may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of a polypeptide of the invention with proteinases such as endoLys-C, endoArg-C, endoGlu-C and staphylococcus V8-protease. The digested fragments can be purified by, for example, high performance liquid chromatographic (HPLC) techniques.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “corresponds to” or “corresponding to” is meant (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

By “derivative” is meant a polypeptide that has been derived from the basic sequence by modification, for example by conjugation or complexing with other chemical moieties or by post-translational modification techniques as would be understood in the art. The term “derivative” also includes within its scope alterations that have been made to a parent sequence including additions or deletions that provide for functional equivalent molecules.

By “effective amount”, in the context of treating or preventing a condition is meant the administration of that amount of active substance to an individual in need of such treatment or prophylaxis, either in a single dose or as part of a series, that is effective for treatment or prophylaxis of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

“Hybridization” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently.

Reference herein to “immuno-interactive” includes reference to any interaction, reaction, or other form of association between molecules and in particular where one of the molecules is, or mimics, a component of the immune system.

By “isolated” is meant material that is substantially or essentially free from components that normally accompany it in its native state.

By “obtained from” is meant that a sample such as, for example, a polynucleotide extract or polypeptide extract is isolated from, or derived from, a particular source of the host. For example, the extract can be obtained from a tissue or a biological fluid isolated directly from the host.

The term “oligonucleotide” as used herein refers to a polymer composed of a multiplicity of nucleotide residues (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotide residues and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule can vary depending on the particular application. An oligonucleotide is typically rather short in length, generally from about 10 to 30 nucleotide residues, but the term can refer to molecules of any length, although the term “polynucleotide” or “nucleic acid” is typically used for large oligonucleotides.

By “operably linked” is meant a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. “Operably linking” a promoter to a polynucleotide is meant placing the polynucleotide (e.g., protein encoding polynucleotide or other transcript) under the regulatory control of a promoter, which then controls the transcription and optionally translation of that polynucleotide. In the construction of heterologous promoter/structural gene combinations, it is generally preferred to position a promoter or variant thereof at a distance from the transcription start site of the polynucleotide, which is approximately the same as the distance between that promoter and the gene it controls in its natural setting; i.e.: the gene from which the promoter is derived. As is known in the art, some variation in this distance can be accommodated without loss of function.

The term “patient” refers to patients of human or other mammal and includes any individual it is desired to examine or treat using the methods of the invention. However, it will be understood that “patient” does not imply that symptoms are present. Suitable mammals that fall within the scope of the invention include, but are not restricted to, primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes, avians, reptiles).

By “pharmaceutically acceptable carrier” is meant a solid or liquid filler, diluent or encapsulating substance that can be safely used in topical or systemic administration to a animal, preferably a mammal including humans.

The term “polynucleotide” or “nucleic acid” as used herein designates mRNA, RNA, cRNA, cDNA or DNA. The term typically refers to oligonucleotides greater than 30 nucleotide residues in length. “Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

By “primer” is meant an oligonucleotide which, when paired with a strand of DNA, is capable of initiating the synthesis of a primer extension product in the presence of a suitable polymerizing agent. The primer is preferably single-stranded for maximum efficiency in amplification but can alternatively be double-stranded. A primer must be sufficiently long to prime the synthesis of extension products in the presence of the polymerization agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotide residues, although it can contain fewer nucleotide residues. Primers can be large polynucleotides, such as from about 200 nucleotide residues to several kilobases or more. Primers can be selected to be “substantially complementary” to the sequence on the template to which it is designed to hybridize and serve as a site for the initiation of synthesis. By “substantially complementary”, it is meant that the primer is sufficiently complementary to hybridize with a target polynucleotide. Preferably, the primer contains no mismatches with the template to which it is designed to hybridize but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridize therewith and thereby form a template for synthesis of the extension product of the primer.

“Probe” refers to a molecule that binds to a specific sequence or sub-sequence or other moiety of another molecule. Unless otherwise indicated, the term “probe” typically refers to a polynucleotide probe that binds to another polynucleotide, often called the “target polynucleotide”, through complementary base pairing. Probes can bind target polynucleotides lacking complete sequence complementarity with the probe, depending on the stringency of the hybridization conditions. Probes can be labeled directly or indirectly.

The term “recombinant polynucleotide” as used herein refers to a polynucleotide formed in vitro by the manipulation of a polynucleotide into a form not normally found in nature. For example, the recombinant polynucleotide can be in the form of an expression vector. Generally, such expression vectors include transcriptional and translational regulatory polynucleotide operably linked to the polynucleotide.

By “recombinant polypeptide” is meant a polypeptide made using recombinant techniques, i.e. through the expression of a recombinant or synthetic polynucleotide.

By “reporter molecule” as used in the present specification is meant a molecule that, by its chemical nature, provides an analytically identifiable signal that allows the detection of a complex comprising an antigen-binding molecule and its target antigen. The term “reporter molecule” also extends to use of cell agglutination or inhibition of agglutination such as red blood cells on latex beads, and the like.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 50 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

The term “sequence identity” as used herein refers to the extent that sequences are identical on a nucleotide-by-nucleotide basis or an amino acid-by-amino acid basis over a window of comparison. Thus, a “percentage of sequence identify” is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e. the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software.

“Similarity” refers to the percentage number of amino acids that are identical or constitute conservative substitutions as described in Section 5.2 infra. Similarity may be determined using sequence comparison programs such as GAP (Deveraux et al. 1984, Nucleic Acids Research 12, 387-395). In this way, sequences of a similar or substantially different length to those cited herein might be compared by insertion of gaps into the alignment, such gaps being determined, for example, by the comparison algorithm used by GAP.

“Stringency” as used herein, refers to the temperature and ionic strength conditions, and presence or absence of certain organic solvents, during hybridization and washing procedures. The higher the stringency, the higher will be the degree of complementarity between immobilized target nucleotide sequences and the labeled probe polynucleotide sequences that remain hybridized to the target after washing.

“Stringent conditions” refers to temperature and ionic conditions under which only nucleotide sequences having a high frequency of complementary bases will hybridize. The stringency required is nucleotide sequence dependent and depends upon the various components present during hybridization and subsequent washes, and the time allowed for these processes. Generally, in order to maximize the hybridization rate, non-stringent hybridization conditions are selected; about 20 to 25° C. lower than the thermal melting point (Tₘ). The Tₘ is the temperature at which 50% of specific target sequence hybridizes to a perfectly complementary probe in solution at a defined ionic strength and pH. Generally, in order to require at least about 85% nucleotide complementarity of hybridized sequences, highly stringent washing conditions are selected to be about 5 to 15° C. lower than the Tₘ. In order to require at least about 70% nucleotide complementarity of hybridized sequences, moderately stringent washing conditions are selected to be about 15 to 30° C. lower than the Tₘ. Highly permissive (low stringency) washing conditions may be as low as 50° C. below the Tₘ, allowing a high level of mis-matching between hybridized sequences. Those skilled in the art will recognize that other physical and chemical parameters in the hybridization and wash stages can also be altered to affect the outcome of a detectable hybridization signal from a specific level of homology between target and probe sequences.

By “vector” is meant a polynucleotide molecule, preferably a DNA molecule derived, for example, from a plasmid, bacteriophage, yeast or virus, into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e. a vector that exists as an extrachromosomal entity, the replication of which is independent of chromosomal replication, e.g. a linear or closed circular plasmid, an extrachromosomal element, a minichromosome, or an artificial chromosome. The vector can contain any means for assuring self-replication. Alternatively, the vector can be one which, when introduced into the host cell, is integrated into the genome and replicated together with the chromosome(s) into which it has been integrated. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. In the present case, the vector is preferably a viral or viral-derived vector, which is operably functional in animal and preferably mammalian cells. Such vector may be derived from a poxvirus, an adenovirus or yeast. The vector can also include a selection marker such as an antibiotic resistance gene that can be used for selection of suitable transformants. Examples of such resistance genes are known to those of skill in the art and include the nptII gene that confers resistance to the antibiotics kanamycin and G418 (Geneticin®) and the hph gene which confers resistance to the antibiotic hygromycin B.

As used herein, underscoring or italicizing the name of a gene shall indicate the gene, in contrast to its protein product, which is indicated in the absence of any underscoring or italicizing. For example, “KLK7” shall mean the KLK7 gene, whereas “K7” shall indicate the protein product of the “KLK7” gene.

2. Polynucleotides

2.1 Aberrant KLK5 Polynucleotides and Aberrantly Expressible KLK7 Polynucleotides

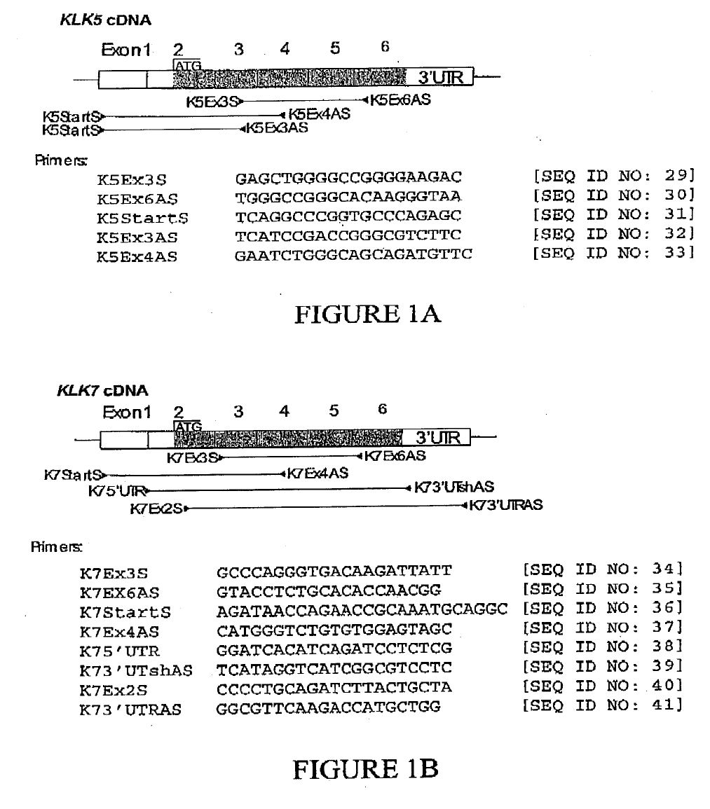

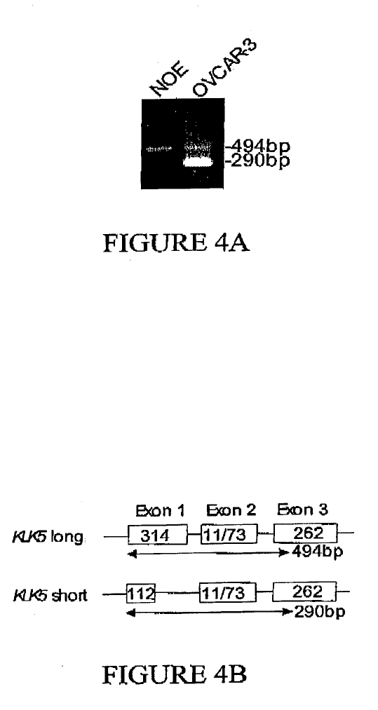

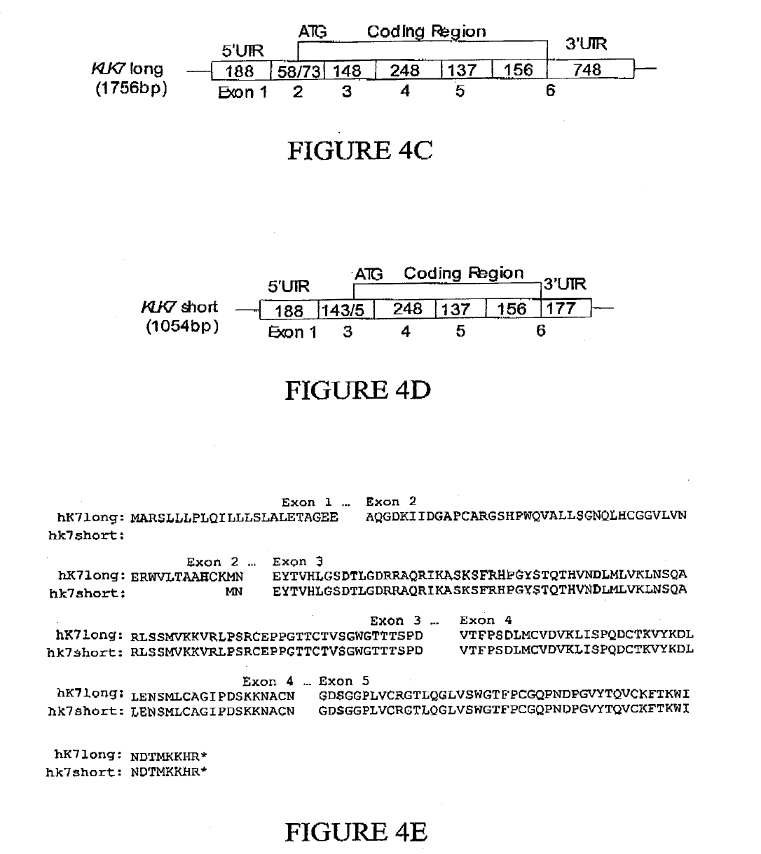

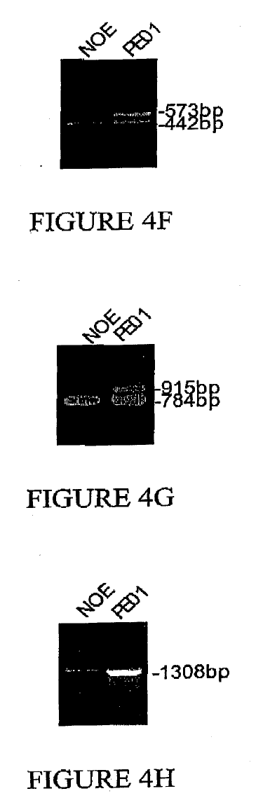

The present invention is predicated in part on the determination that alterations, inclusive of substitutions, deletions and additions, within the transcribable regions of KLK5, correlate with the presence or risk of a cancer or related condition. The invention, therefore, provides in one aspect an isolated polynucleotide comprising a nucleotide sequence which corresponds or is complementary to at least a portion of an aberrant KLK5 polynucleotide that correlates with the presence or risk of a cancer or related condition. The aberrant KLK5 polynucleotide may be distinguished from a normal KLK5 polynucleotide by the substitution, deletion or addition of at least one nucleotide. Such substitution, addition or deletion can reside anywhere in KLK5 or transcript thereof, and preferably in an untranslated region (UTR) of KLK5, and more preferably in a 5′ UTR of KLK5. In a preferred embodiment, the aberrant KLK5 polynucleotide is selected from the group consisting of: (a) a polynucleotide comprising the entire sequence of nucleotides set forth in SEQ ID NO: 9; and (b) a polynucleotide fragment of (a), wherein the fragment comprises at least 4, 5, 6, 7, 8, 9, 10, 12, 14, 16, 18, 20, 25, 30, 40, 50 or 100 contiguous nucleotides which include nucleotides 106 and 107 of SEQ ID NO: 9. SEQ ID NO: 9 corresponds to an alternately spliced variant of KLK5, which is present in cancer cells but not in normal cells (FIG. 4A) and which comprises a deletion of a region corresponding to known exon 1 of normal KLK5 as set forth in SEQ ID NO: 1 (see FIG. 4B). However, the present inventors have found an additional exon that codes extra 5′UTR, which is located further upstream of this known exon, and which now defines a new KLK5 exon 1. Consistent with this definition, known exons 1 to 5 of KLK5 have been redefined as new exons 2 to 6, respectively (see FIGS. 1A and 4B). The region, which is deleted in SEQ ID NO: 9, consists essentially of the sequence set forth in SEQ ID NO: 5 and results in a truncated 5′UTR, which is about 202 nucleotides smaller than the long form of KLK5 presented in SEQ ID NO: 8 that is found in both normal and cancer cells (see FIG. 4A).

The present invention also features a KLK7 polynucleotide that is aberrantly expressible between normal and cancer cells. In particular, the aberrantly expressible KLK7 polynucleotide is expressed at a higher level and/or functional activity than the normal reference level and/or functional activity. Suitably, a KLK7 expression product is aberrantly expressed if its level and/or functional activity is at least 110%, 150%, 200%, 300%, 500% or 1000% of the level and/or functional activity of that expression product in normal cells. In a preferred embodiment, the aberrantly expressible KLK7 polynucleotide is selected from the group consisting of: (a) a polynucleotide comprising the entire sequence of nucleotides as set forth in SEQ ID NO: 16; and (b) a polynucleotide fragment of (a), wherein the fragment comprises at least a portion of KLK7 exon 2, whose sequence is set forth for example in SEQ ID NO: 20. SEQ ID NO: 16 corresponds to a long form of KLK7. This long KLK7 transcript has 6 exons and contains a longer exon 1, which extends 144 nucleotides further upstream compared with the KLK7 gene sequence published in GenBank Accession No. AF332583, but the coding exons are consistent with this previously published sequence. The 3′ UTR of this long form of KLK7 (748 nts) is smaller than a splice variant of KLK7 (1039 nts; SEQ IN NO: 15) disclosed in GenBank Accession No. 332583 but longer than another KLK7 splice variant (190 nts; SEQ IN NO: 13) disclosed in the same reference. The ATG codon is located at nucleotide 247 of SEQ ID NO: 16 and a protein, hK7, of 253 amino acids is predicted (FIG. 4E; SEQ ID NO: 17) that is identical to the enzyme purified from human skin (16). In comparison to the long form of KLK7, the present inventors have also identified a shorter KLK7 mRNA transcript (1054 nts, FIG. 4D) whose sequence is presented in SEQ ID NO: 18, and which contains 5 exons with exon 2 deleted and with only 177 bp in the 3′UTR region. The short KLK7 transcript generates a protein of 181 amino acids, has a different protein translation start site (ATG at nt 332 of SEQ ID NO: 18) is utilized, and encodes a shortened protein sequence without a pre and pro region (FIG. 4E). The truncated hK7 does not contain the histidine residue of the catalytic triad (FIG. 4E). Both short and long KLK7 transcripts were expressed by cancer cells or cell lines although some cancer cells/cell lines predominantly expressed the longer transcript (FIG. 4F-H). Not wishing to be bound by any one particular theory or mode of operation, it is proposed that, in contrast to the shorter KLK7 transcript, the long KLK7 transcript would encode the full length pre- pro- chymotryptic enzyme enabling secreted active hK7 enzyme to be present in the cancer cells but not normal ovarian epithelial cells.

Although the present invention has been described with reference to certain preferred embodiments, it will be understood that the invention contemplates any isolated aberrant KLK5 polynucleotide or aberrantly expressible KLK7 polynucleotide that correlates with the presence or risk of a cancer or related condition, other than those described above. Such aberrant KLK5 polynucleotides or aberrantly expressible KLK7 polynucleotides may be obtained from individuals afflicted with a cancer or related condition. In one embodiment, the cancer is regulatable by a hormone including, but not restricted to, testosterone, estrogen and progesterone. For example, the cancer may be selected from ovarian, endometrial or prostate cancer. In a preferred embodiment, the cancer is a serous cancer, which is preferably a serous carcinoma such as ovarian cancer.

Nucleic acid isolation protocols are well known to those of skill in the art. For example, aberrant KLK5 polynucleotide or aberrantly expressible KLK7 polynucleotide may be prepared according to the following procedure:

(a) creating primers comprising a portion of a reference KLK5 or KLK7 polynucleotide;

(b) obtaining a nucleic acid extract from an individual affected with a cancer or related condition; and

(c) using the primers to amplify, via nucleic acid amplification techniques, at least one amplification product from the nucleic acid extract, wherein the amplification product corresponds to an aberrant KLK5 polynucleotide or fragment thereof or to a KLK7 polynucleotide which is aberrantly expressed in the affected individual.

Suitable nucleic acid amplification techniques are well known to the skilled addressee, and include polymerase chain reaction (PCR) as for example described in Ausubel et al. (supra); strand displacement amplification (SDA) as for example described in U.S. Pat. No. 5,422,252; rolling circle replication (RCR) as for example described in Liu et al., (1996, J. Am. Chem. Soc. 118:1587-1594 and International application WO 92/01813) and Lizardi et al., (International Application WO 97/19193); nucleic acid sequence-based amplification (NASBA) as for example described by Sooknanan et al., (1994, Biotechniques 17:1077-1080); and Q-β replicase amplification as for example described by Tyagi et al., (1996, Proc. Natl. Acad. Sci. USA 93:5395-5400).

2.2 Polynucleotide Variants

The present invention also encompasses polynucleotide variants displaying substantial sequence identity with a reference polynucleotide as broadly described above, which correlates with the presence or risk of a cancer or related condition. In one embodiment, the variant polynucleotides comprise the same alteration as a reference aberrant polynucleotide, or an alteration that encodes the same aberrant amino acid(s) (silent alteration) encoded by the reference polynucleotide. In another embodiment, the variant polynucleotides are distinguished from a reference polynucleotide that is aberrantly expressed in a cancer cell by the addition, deletion and/or substitution of at least one nucleotide. Preferably such variant polynucleotides are functionally similar to the reference polynucleotide. Also encompassed are aberrant polynucleotide variants that hybridize with a reference aberrant polynucleotide sequence under stringent conditions that are defined hereinafter. Practitioners in the art will recognize that in view of the degeneracy in the genetic code, silent alterations to a reference aberrant polynucleotide can be made to provide a synonymous polynucleotide encoding the same polypeptide as the reference aberrant polynucleotide.

Typically, polynucleotide variants that are substantially complementary to a reference polynucleotide are identified by blotting techniques that include a step whereby nucleic acids are immobilized on a matrix (preferably a synthetic membrane such as nitrocellulose), followed by a hybridization step, and a detection step. Southern blotting is used to identify a complementary DNA sequence; northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al. (1994-1998, supra) at pages 2.9.1 through 2.9.20.

It will be understood that polynucleotide variants according to the invention will hybridize to a reference polynucleotide under at least low stringency conditions. Reference herein to low stringency conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature.

Suitably, the polynucleotide variants hybridize to a reference polynucleotide under at least medium stringency conditions. Medium stringency conditions include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 60-65° C.

Preferably, the polynucleotide variants hybridize to a reference polynucleotide under high stringency conditions. High stringency conditions include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C.

Other stringent conditions are well known in the art. A skilled addressee will recognize that various factors can be manipulated to optimize the specificity of the hybridization. Optimization of the stringency of the final washes can serve to ensure a high degree of hybridization. For detailed examples, see Ausubel et al., supra at pages 2.10.1 to 2.10.16 and Sambrook et al. (1989, supra) at sections 1.101 to 1.104.

While stringent washes are typically carried out at temperatures from about 42° C. to 68° C., one skilled in the art will appreciate that other temperatures may be suitable for stringent conditions. Maximum hybridization rate typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel et al., supra at page 2.10.8).

In general, the Tₘ of a perfectly matched duplex of DNA may be predicted as an approximation by the formula:
Tₘ=81.5+16.6 (log₁₀M)+0.41 (%G+C)−0.63 (% formamide)−(600/length)

wherein: M is the concentration of Na⁺, preferably in the range of 0.01 molar to 0.4 molar; %G+C is the sum of guanosine and cytosine bases as a percentage of the total number of bases, within the range between 30% and 75% G+C; % formamide is the percent formamide concentration by volume; length is the number of base pairs in the DNA duplex.

The Tₘ of a duplex DNA decreases by approximately 1° C. with every increase of 1% in the number of randomly mismatched base pairs. Washing is generally carried out at Tₘ−15° C. for high stringency, or Tₘ−30° C. for moderate stringency.

In one example of a hybridization procedure, a membrane (e.g., a nitrocellulose membrane or a nylon membrane) containing immobilized DNA is hybridized overnight at 42° C. in a hybridization buffer (50% deionised formamide, 5×SSC, 5× Denhardt's solution (0.1% ficoll, 0.1% polyvinylpyrollidone and 0.1% bovine serum albumin), 0.1% SDS and 200 mg/mL denatured salmon sperm DNA) containing labeled probe. The membrane is then subjected to two sequential medium stringency washes (i.e., 2×SSC, 0.1% SDS for 15 min at 45° C., followed by 2×SSC, 0.1% SDS for 15 min at 50° C.), followed by two sequential higher stringency washes (i.e., 0.2×SSC, 0.1% SDS for 12 min at 55° C. followed by 0.2×SSC and 0.1%SDS solution for 12 min at 65-68° C.

Methods for detecting a labeled polynucleotide hybridized to an immobilized polynucleotide are well known to practitioners in the art. Such methods include autoradiography, phosphorimaging, and chemiluminescent, fluorescent and colorimetric detection.

3. Vectors

The invention also contemplates a vector comprising a polynucleotide according to the invention. Vectors contemplated by the present invention include cloning vectors and expression vectors.

In one embodiment, a polynucleotide of the invention is suitably rendered expressible in a host cell by operably linking the polynucleotide with a regulatory polynucleotide. The synthetic construct or vector thus produced may be introduced firstly into an organism or part thereof before subsequent expression of the construct in a particular cell or tissue type. Any suitable organism is contemplated by the invention, which may include unicellular as well as multi-cellular organisms. Suitable unicellular organisms include bacteria. Exemplary multi-cellular organisms include yeast, mammals and plants.

The construction of the vector may be effected by any suitable technique as for example described in the relevant sections of Ausubel et al. (supra) and Sambrook et al. (supra). However, it should be noted that the present invention is not dependent on and not directed to any one particular technique for constructing the vector.

Regulatory polynucleotides which may be utilized to regulate expression of the polynucleotide include, but are not limited to, a promoter, an enhancer, and a transcriptional terminator. Such regulatory sequences are well known to those of skill in the art. Suitable promoters that may be utilized to induce expression of the polynucleotides of the invention include constitutive promoters and inducible promoters.

4. Host Cells and Cell Lines

The invention also encompasses a host cell comprising a vector as broadly described above, as well as a cell line that comprises a polynucleotide comprising a nucleotide sequence which corresponds or is complementary to at least a portion of an aberrant KLK5 polynucleotide, or an aberrantly expressible KLK7 polynucleotide, that correlates with the presence or risk of a cancer or related condition. The cell line is preferably produced from a cell of an individual who is afflicted with the cancer or related condition, wherein the cell comprises an aberrant KLK5 polynucleotide, or an aberrantly expressible KLK7 polynucleotide, that has the correlation. Many methods of producing the cell line are known to those of skill in the art. Suitably, the cell line is obtained by immortalization of a cell with Epstein-Barr virus as is known in the art.

5. Polypeptides

5.1 K7 Polypeptides

The present invention also encompasses an isolated polypeptide comprising an amino acid sequence which corresponds to at least a portion of an aberrantly expressible K7 polypeptide that correlates with the presence or risk of a cancer or related condition. Preferably, the aberrantly expressible K7 polypeptide comprises all or part of the amino acid sequence set forth in SEQ ID NO: 21. More preferably, the aberrantly expressible K7 polypeptide comprises the sequence set forth in SEQ ID NO: 17.

5.2 K7 Polypeptide Variants

With regard to variant polypeptides of the invention, it will be understood that such variants should retain antigenic or immunogenic activity of the parent or reference polypeptide, which includes the production of elements that specifically bind to the amino acid sequence which corresponds to at least a portion of an aberrantly expressible K7 polypeptide (e.g., SEQ ID NO: 21 or fragment thereof) that correlates with the presence or risk of a cancer or related condition. Such variant polypeptides, therefore, constitute immuno-mimetics, which mimic the immunogenicity or antigenicity of a reference variant polypeptide. A conservative amino acid substitution is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Substantial changes in function are made by selecting substitutions that are less conservative than those mentioned above. Other replacements would be non-conservative substitutions and relatively fewer of these may be tolerated. Generally, the substitutions which are likely to produce the greatest changes in a polypeptide's properties are those in which (a) a hydrophilic residue (e.g. Ser or Thr) is substituted for, or by, a hydrophobic residue (e.g. Ala, Leu, Ile, Phe or Val); (b) a cysteine or proline is substituted for, or by, any other residue; (c) a residue having an electropositive side chain (e.g. Arg, His or Lys) is substituted for, or by, an electronegative residue (e.g. Glu or Asp) or (d) a residue having a bulky side chain (e.g. Phe or Trp) is substituted for, or by, one having a smaller side chain (e.g. Ala, Ser)or no side chain (e.g. Gly).

In general, variants comprise regions that are at least 75% similar, more suitably at least 80%, preferably at least 85%, and most preferably at least 90% similar to the basic sequence as for example shown in SEQ ID NO: 17. In an alternate embodiment, variants comprise regions that have at least 60%, more suitably at least 70%, preferably at least 80%, and most preferably at least 90% identity over a parent amino acid sequence of identical size (“comparison window”) or when compared to an aligned sequence in which the alignment is performed by a computer homology program known in the art.

5.3 K7 Polypeptide Derivatives

Suitable polypeptide derivatives include amino acid deletions and/or additions to a reference aberrantly expressible K7 polypeptide according to the invention such as but not limited to SEQ ID NO: 17, or variants thereof, wherein the derivatives retain antigenic or immunogenic activity, which includes the production of elements that specifically bind to the reference polypeptide. “Additions” of amino acids may include fusion of the aberrantly expressible polypeptides, fragments thereof or variants of these with other polypeptides or proteins. In this regard, it will be appreciated that the aberrantly expressible polypeptides, polypeptide fragments or variants of the invention may be incorporated into larger polypeptides, and such larger polypeptides may also be expected to retain the antigenic or immunogenic activity mentioned above.

6. Antigen-Binding Molecules

The invention also contemplates one or more antigen-binding molecules which bind to the aforementioned polypeptides, polypeptide fragments, variants and derivatives and which provide specificity for their detection. For example, the antigen-binding molecules may comprise whole polyclonal antibodies. Such antibodies may be prepared, for example, by injecting a polypeptide, fragment, variant or derivative of the invention into a production species, which may include mice or rabbits, to obtain polyclonal antisera. Methods of producing polyclonal antibodies are well known to those skilled in the art. Exemplary protocols which may be used are described for example in Coligan et al., CURRENT PROTOCOLS IN IMMUNOLOGY, (John Wiley & Sons, Inc, 1991), and Ausubel et al., (1994-1998, supra), in particular Section III of Chapter 11.

In lieu of the polyclonal antisera obtained in the production species, monoclonal antibodies may be produced using the standard method as described, for example, by Kohler and Milstein (1975, Nature 256, 495-497), or by more recent modifications thereof as described, for example, in Coligan et al., (1991, supra) by immortalizing spleen or other antibody-producing cells derived from a production species which has been inoculated with one or more of the polypeptides, polypeptide fragments, variants or derivatives of the invention.

The invention also contemplates as antigen-binding molecules Fv, Fab, Fab′ and F(ab′)₂ immunoglobulin fragments or other synthetic antigen-binding molecules such as synthetic stabilized Fv fragments, dAbs, minibodies and the like, which can be produced using routine methods by practitioners in the art.

Suitably, the antigen-binding molecule binds to a portion of a K7 polynucleotide according to the invention, which correlates with the presence or risk of a cancer or related condition. In a preferred embodiment of this type, the antigen-binding molecule binds specifically to SEQ ID NO: 21 or fragment thereof.

The antigen-binding molecules of the invention may be used for affinity chromatography in isolating a natural or recombinant polypeptide or biologically active fragment of the invention. For example reference may be made to immunoaffinity chromatographic procedures described in Chapter 9.5 of Coligan et al., (1995-1997, supra). The antigen-binding molecules can be used to screen expression libraries for variant polypeptides of the invention as described herein. They can also be used to detect polypeptides, polypeptide fragments, variants and derivatives of the invention as described hereinafter.

7. Methods of Detecting Aberrant Expression of KLK5/KLK7 or K7 Molecules

7.1 Assays for Detecting Modulation of the Level and/or Functional Activity of K7

The present invention is predicated in part on the discovery that aberrant KLK5 polynucleotides are expressed in cancers but not in normal tissues and that certain KLK7 mRNAs and their encoded products are expressed at a higher level and/or functional activity in cancers than in normal tissues. Thus, the invention also features a method for detecting the presence or diagnosing the risk of a cancer or related condition in a patient, comprising detecting the presence of an aberrant KLK5 expression product or the aberrant expression of a KLK7 expression product in a biological sample obtained from the patient, wherein the aberrant expression product or the aberrant expression correlates with the presence or risk of the cancer or related condition.

In one embodiment, the method comprises detecting a change in the level and/or functional activity of an expression product of KLK5 or KLK7. Preferably, the method comprises detecting an increase in the level and/or functional activity of the expression product relative to the corresponding normal reference level and/or functional activity. Suitably, the level and/or functional activity of the expression product in the biological sample is at least 110%, more preferably at least 200%, even more preferably at least 300%, even more preferably at least 500%, even more preferably at least 1000%, even more preferably at least 2000%, even more preferably at least 4000%, even more preferably at least 6000%, even more preferably at least 8000%, and still more preferably at least 10,000% of that which is present in a corresponding biological sample obtained from a normal individual or from an individual who is not afflicted with the condition.

Any method of directly or indirectly detecting modulation in the level and/or functional activity of the expression product is encompassed by the present invention. For example, such detection can be achieved utilizing techniques including, but not restricted to, immunoassays such as Western blotting and ELISAs, and RT-PCR. Exemplary immunoassays, which could be used for these purposes, are described for example in Section 7.2. For example, in one embodiment, a biological sample from a patient is contacted with an antigen-binding molecule that is specifically immuno-interactive with a K7 polypeptide which is aberrantly expressible in a cancer-affected individual. The concentration of a complex comprising the polypeptide and the antigen-binding molecule is measured in the contacted sample and the measured complex concentration is then related to the concentration of the polypeptide in the sample. Preferably, the concentration of the polypeptide is compared to a reference or baseline level of the polypeptide corresponding to normal tissues. The presence of the cancer or related condition is diagnosed if the concentration of the polypeptide corresponds to a non-reference level concentration.

7.2 Detection of Normal and Aberrant Molecules

It is to be understood that although the following discussion is specifically directed to human patients, the teachings are also applicable to any animal that expresses an aberrant KLK5 polynucleotide which corresponds to a defect in KLK5 gene or transcript structure, such that clinical manifestations particularly those seen in patients with a cancer or related condition are found. It will also be appreciated that the methods described herein are applicable to any patient suspected of developing, or having, a the condition, whether such condition is manifest at a young age or at a more advanced age in a patient's life.

Thus, the screening of a patient for the presence or risk of a cancer or related condition is now be possible by detecting an aberrant KLK5 polynucleotide that correlates with that presence or risk. The screening method of the invention allows a presymptomatic diagnosis, including prenatal diagnosis, for the presence of an aberrant KLK5 gene or transcript thereof in a patient and thus the basis for an opinion concerning the likelihood that that patient would develop or has developed a cancer or related condition or symptoms thereof. For example, in the method of screening, a tissue sample can be taken from a patient, and screened for the presence of one or more normal KLK5 polynucleotides. The normal human KLK5 polynucleotides can be characterized based upon, for example, detection of restriction digestion patterns in ‘normal’ versus the patient's DNA, including Restriction Fragment Length Polymorphism (RFLP) analysis, using nucleic acid probes prepared against the normal KLK5 gene(s) (or functional fragments thereof). Similarly, KLK5 mRNA may be characterized and compared to normal KLK5 mRNA levels and/or size as found in human population not at risk of developing a cancer or related condition using similar probes.

An aberrant KLK5 polynucleotide may be detected by determining the sequence of KLK5 genomic DNA or CDNA obtained or derived from a patient and comparing the sequence to that of wild-type KLK5 DNA or transcripts or to aberrant KLK5 transcripts described herein to thereby determine whether the sequence corresponds to an aberrant KLK5 polynucleotide. Alternatively, a nucleic acid extract from a patient may be utilized in concert with oligonucleotide primers corresponding to sense and antisense sequences of an aberrant polynucleotide sequence under test, or flanking sequences thereof, in a nucleic acid amplification reaction such as PCR, or the ligase chain reaction (LCR) as for example described in International Application WO89/09385. A variety of automated solid-phase detection techniques are also appropriate. For example, very large scale immobilized primer arrays (VLSIPS™) are used for the detection of nucleic acids as for example described by Fodor et al., (1991, Science 251:767-777) and Kazal et al., (1996, Nature Medicine 2:753-759). The above generic techniques are well known to persons skilled in the art. Preferably, at least one of the primers is an allele-specific primer specific for the aberrant polynucleotide under test. Accordingly, the present invention in another aspect contemplates a probe for interrogating nucleic acid for the presence of an aberrant KLK5 polynucleotide associated with at least one condition selected from a cancer or a benign tumor, comprising a nucleotide sequence which corresponds or is complementary to a portion of an aberrant KLK5 polynucleotide that correlates with the presence or risk of a cancer or related condition.

Alternatively, the presence or absence of a restriction endonuclease cleavage site resulting from a mutation or aberrant splicing in the normal KLK5 polynucleotide may be taken advantage by subjecting the aberrant polynucleotide to digestion with the restriction endonuclease. Accordingly, the present invention includes and encompasses detecting an aberrant KLK5 polynucleotide by RFLP analysis.

Alternatively, allele specific oligonucleotide primers may be used in PCR or LCR assays to detect an aberrant KLK5 polynucleotide as described above. For example, a sense primer specific for a normal KLK5 allele or transcript, a sense primer specific for an aberrant KLK5 allele or transcript may be used together with a common antisense primer. Alternatively, the two allele/transcript specific primers may be used in concert with another preferably abutting sense primer, which is complementary to a target sequence immediately adjacent and downstream of the target sequences of the allele/transcript specific primers, in LCR, in the Oligonucleotide Ligation Assay (OLA) as for example described by Landegren et al., 1988, Science 241 1077-1080.

Alternatively, a nucleic acid polymorphism in KLK5 may be detected using first-nucleotide change technology described by Dale et al. in U.S. Pat. No. 5,856,092.

The presence in the biological sample of an aberrant KLK5 size pattern, such as an aberrant KLK5 RFLP, and/or aberrant KLK5 mRNA sizes or levels and/or aberrant KLK5 polynucleotide linked to a condition described herein would indicate that the patient has developed or is at risk of developing a symptom associated with that condition.

The diagnostic and screening methods of the invention are especially useful for a patient suspected of being at risk of developing a cancer or related condition based on family history, or a patient in which it is desired to diagnose or eliminate the presence of that condition as a causative agent underlying a patient's symptoms. Prenatal diagnosis can be performed when desired, using any known method to obtain fetal cells, including amniocentesis, chorionic villous sampling (CVS), and foetoscopy.

8. Detection Kits

The present invention also provides kits for the detection in a biological sample of an aberrantly expressible K7 polypeptide or fragment thereof, or of an aberrant KLK5 polynucleotide or fragment thereof. These will contain one or more particular agents described above depending upon the nature of the test method employed. In this regard, the kits may include one or more of an aberrantly expressible polypeptide or fragments thereof, an aberrant polynucleotide or fragment thereof, variants or derivative of these molecules, a nucleic acid probe, or an antigen-binding molecule, as broadly described above. The kits may also optionally include appropriate reagents for detection of labels, positive and negative controls, washing solutions, dilution buffers and the like. For example, a nucleic acid-based detection kit may include (i) a polynucleotide according to the invention (which may be used as a positive control), (ii) an oligonucleotide primer according to the invention, and optionally a DNA polymerase, DNA ligase etc depending on the nucleic acid amplification technique employed.

9. Identification of Target Molecule Modulators

The invention also features a method of screening for agents that can modulate the expression of a gene or the level and/or functional activity of a wild-type or aberrant expression product of the gene, wherein the gene is selected from KLK5, KLK7 or a gene belonging to the same regulatory or biosynthetic pathway as KLK5 or KLK7. In accordance with the present invention, agents that modulate these target molecules are useful for treating and/or preventing a cancer or related condition or for restoring a normal level and/or functional activity of a KLK5 or KLK7 expression product. The screening method comprises contacting a preparation comprising at least a portion of a wild-type or aberrant polypeptide encoded by the gene, or a variant or derivative thereof, or a genetic sequence that modulates the expression of the gene, with the agent and detecting a change in the level and/or functional activity of the polypeptide or variant or derivative, or of a product expressed from the genetic sequence.

Screening for modulatory agents according to the invention can be achieved by any suitable method. For example, the method may include contacting a cell comprising at least a portion of an aberrant KLK5 expression product or at least a portion of an aberrantly expressible KLK7 expression product, which correlate wit the presence or risk of a cancer or related condition, with an agent suspected of having the modulatory activity and screening for the modulation of the level and/or functional activity of the expression product or for the modulation of the activity or expression of a downstream cellular target of the protein or the expression product. Detecting such modulation can be achieved utilizing techniques including, but not restricted to, ELISA, cell-based ELISA, filter-binding ELISA, inhibition ELISA, Western blots, immunoprecipitation, slot or dot blot assays, immunostaining, RIA, scintillation proximity assays, fluorescent immunoassays using antigen-binding molecule conjugates or antigen conjugates of fluorescent substances such as fluorescein or rhodamine, Ouchterlony double diffusion analysis, immunoassays employing an avidin-biotin or a streptavidin-biotin detection system, and nucleic acid detection assays including reverse transcriptase polymerase chain reaction (RT-PCR).

It will be understood that a polynucleotide from which a target molecule of interest is regulated or expressed may be naturally occurring in the cell which is the subject of testing or it may have been introduced into the host cell for the purpose of testing. Further, the naturally-occurring or introduced sequence may be constitutively expressed—thereby providing a model useful in screening for agents which down-regulate expression of an encoded product of the sequence wherein the down regulation can be at the nucleic acid or expression product level—or may require activation—thereby providing a model useful in screening for agents that up-regulate expression of an encoded product of the sequence. Further, to the extent that a polynucleotide is introduced into a cell, that polynucleotide may comprise the entire coding sequence which codes for a target protein or it may comprise a portion of that coding sequence (e.g. a domain such as a protein binding domain such as the pre- pro- region of the long form of K7 set forth in SEQ ID NO: 17) or a portion that regulates expression of a product encoded by the polynucleotide (e.g., a promoter). For example, the promoter that is naturally associated with the polynucleotide may be introduced into the cell that is the subject of testing. In this regard, where only the promoter is utilized, detecting modulation of the promoter activity can be achieved, for example, by operably linking the promoter to a suitable reporter polynucleotide including, but not restricted to, green fluorescent protein (GFP), luciferase, β-galactosidase and catecholamine acetyl transferase (CAT). Modulation of expression may be determined by measuring the activity associated with the reporter polynucleotide.

In another example, the subject of detection could be a downstream regulatory target of the target molecule, rather than target molecule itself or the reporter molecule operably linked to a promoter of a gene encoding a product the expression of which is regulated by the target protein.

These methods provide a mechanism for performing high throughput screening of putative modulatory agents such as proteinaceous or non-proteinaceous agents comprising synthetic, combinatorial, chemical and natural libraries. These methods will also facilitate the detection of agents which bind either the polynucleotide encoding the target molecule or which modulate the expression of an upstream molecule, which subsequently modulates the expression of the polynucleotide encoding the target molecule. Accordingly, these methods provide a mechanism of detecting agents that either directly or indirectly modulate the expression and/or activity of a target molecule according to the invention.

In a series of preferred embodiments, the present invention provides assays for identifying small molecules or other compounds (ie., modulatory agents) which are capable of inducing or inhibiting the level and/or or functional activity of target molecules according to the invention. The assays may be performed in vitro using non-transformed cells, immortalised cell lines, or recombinant cell lines. In addition, the assays may detect the presence of increased or decreased expression of genes or production of proteins on the basis of increased or decreased mRNA expression (using, for example, the nucleic acid probes disclosed herein), increased or decreased levels of protein products (using, for example, the antigen binding molecules disclosed herein), or increased or decreased levels of expression of a reporter gene (e.g., GFP, β-galactosidase or luciferase) operatively linked to a target molecule-related gene regulatory region in a recombinant construct.

Thus, for example, one may culture cells which produce a particular target molecule and add to the culture medium one or more test compounds. After allowing a sufficient period of time (e.g., 6-72 hours) for the compound to induce or inhibit the level and/or functional activity of the target molecule, any change in the level from an established baseline may be detected using any of the techniques described above and well known in the art. In particularly preferred embodiments, the cells are epithelial cells. Using the nucleic acid probes and/or antigen-binding molecules disclosed herein, detection of changes in the level and or functional activity of a target molecule, and thus identification of the compound as agonist or antagonist of the target molecule, requires only routine experimentation.

In particularly preferred embodiments, a recombinant assay is employed in which a reporter gene encoding, for example, GFP, β-galactosidase or luciferase is operably linked to the 5′ regulatory regions of a target molecule related gene. Such regulatory regions may be easily isolated and cloned by one of ordinary skill in the art in light of the present disclosure. The reporter gene and regulatory regions are joined in-frame (or in each of the three possible reading frames) so that transcription and translation of the reporter gene may proceed under the control of the regulatory elements of the target molecule related gene. The recombinant construct may then be introduced into any appropriate cell type although mammalian cells are preferred, and human cells are most preferred. The transformed cells may be grown in culture and, after establishing the baseline level of expression of the reporter gene, test compounds may be added to the medium. The ease of detection of the expression of the reporter gene provides for a rapid, high throughput assay for the identification of agonists or antagonists of the target molecules of the invention.

Compounds identified by this method will have potential utility in modifying the expression of target molecule related genes in vivo. These compounds may be further tested in the animal models to identify those compounds having the most potent in vivo effects. In addition, as described above with respect to small molecules having target polypeptide binding activity, these molecules may serve as “lead compounds” for the further development of pharmaceuticals by, for example, subjecting the compounds to sequential modifications, molecular modeling, and other routine procedures employed in rational drug design.

In another embodiment, a method of identifying agents that inhibit long form K7 activity is provided in which a purified preparation of this long form is contacted with a candidate agent, and the level of long form K7 activity is measured by a suitable assay. For example, a long form K7 inhibitor can be identified by measuring the ability of a candidate agent to decrease long form K7 activity in a cell (e.g., an ovarian cell). In this method, a cell that is capable of expressing KLK7 is exposed to, or cultured in the presence and absence of, the candidate agent under conditions in which long form K7 is active in the cell, and an activity selected from the group consisting of tumorigenesis is detected. An agent tests positive if it inhibits any of these activities.

In a preferred embodiment, the agent, which is identifiable for example by the above methods, inhibits, abrogates or otherwise reduces the expression of a gene or the level and/or functional activity of an aberrant or wild-type expression product of the gene, wherein the gene is selected from KLK5 or KLK7 or a gene belonging to the same regulatory or biosynthetic pathway as KLK4 or KLK7, for the treatment and/or prophylaxis of a cancer or related condition. For example, agents that may be used to reduce or abrogate gene expression include, but are not restricted to, oligoribonucleotide sequences, including anti-sense RNA and DNA molecules and ribozymes, that function to inhibit the translation, for example, of KLK4-encoding mRNA. Anti-sense RNA and DNA molecules act to directly block the translation of mRNA by binding to targeted mRNA and preventing protein translation. In regard to antisense DNA, oligodeoxyribonucleotides derived from the translation initiation site, e.g., between −10 and +10 regions of an KLK4 gene, are preferred.

Ribozymes are enzymatic RNA molecules capable of catalyzing the specific cleavage of RNA. The mechanism of ribozyme action involves sequence specific hybridization of the ribozyme molecule to complementary target RNA, followed by a endonucleolytic cleavage. Within the scope of the invention are engineered hammerhead motif ribozyme molecules that specifically and efficiently catalyze endonucleolytic cleavage of KLK5 or KLK7 RNA sequences. Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

Both anti-sense RNA and DNA molecules and ribozymes may be prepared by any method known in the art for the synthesis of nucleic acid molecules. These include techniques for chemically synthesizing oligodeoxyribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesise antisense RNA constitutively or inducibly, depending on the promoter used, can be introduced stably into cell lines.

Various modifications to nucleic acid molecules may be introduced as a means of increasing intracellular stability and half-life. Possible modifications include but are not limited to the addition of flanking sequences of ribo- or deoxy-nucleotides to the 5′ and/or 3′ ends of the molecule or the use of phosphorothioate or 2′ O-methyl rather than phosphodiesterase linkages within the oligodeoxyribonucleotide backbone.

10. Therapeutic and Prophylactic Uses

A further feature of the invention is the use of modulatory agents according to Section 9 as active ingredients (“therapeutic agents”) in pharmaceutical compositions for modulating the level and/or functional activity of a KLK5 or KLK7 expression product; and/or for treatment or prophylaxis of a cancer or related condition. Also encompassed is a method for modulating the level of a KLK5 or KLK7 expression product to a patient in need of such treatment, comprising administering to the patient an effective amount of an agent as broadly described in Section 9 in the presence or absence of a pharmaceutically acceptable carrier. In a preferred embodiment, the patient has an elevated level of the expression product relative to the normal level and the administered agent reduces the level and or functional activity of the KLK5 or KLK7 expression product.

A pharmaceutical composition according to the invention is administered to a patient, preferably prior to such symptomatic state associated with the condition(s). The therapeutic agent present in the composition is provided for a time and in a quantity sufficient to treat that patient. Suitably, the pharmaceutical composition comprises a pharmaceutically acceptable carrier. Depending upon the particular route of administration, a variety of pharmaceutically acceptable carriers, well known in the art may be used. These carriers may be selected from a group including sugars, starches, cellulose and its derivatives, malt, gelatin, talc, calcium sulfate, vegetable oils, synthetic oils, polyols, alginic acid, phosphate buffered solutions, emulsifiers, isotonic saline, and pyrogen-free water. Any suitable route of administration may be employed for providing a patient with the composition of the invention. For example, oral, rectal, parenteral, sublingual, buccal, intravenous, intra-articular, intramuscular, intra-dermal, subcutaneous, inhalational, intraocular, intraperitoneal, intracerebroventricular, transdermal and the like may be employed.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Pharmaceutical compositions of the present invention suitable for oral or parenteral administration may be presented as discrete units such as capsules, sachets or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more immunogenic agents as described above with the carrier which constitutes one or more necessary ingredients. In general, the compositions are prepared by uniformly and intimately admixing the immunogenic agents of the invention with liquid carriers or finely divided solid carriers or both, and then, if necessary, shaping the product into the desired presentation.

The above compositions may be administered in a manner compatible with the dosage formulation, and in such amount as is therapeutically-effective to alleviate patients from symptoms related to the condition(s), or in amounts sufficient to protect patients from developing symptoms related to the condition(s). The dose administered to a patient, in the context of the present invention, should be sufficient to effect a beneficial response in a patient over time such as the therapeutic or prophylactic effects mentioned above. The quantity of the therapeutic agent(s) to be administered may depend on the subject to be treated inclusive of the age, sex, weight and general health condition thereof. In this regard, precise amounts of the therapeutic agent(s) for administration will depend on the judgement of the practitioner. In determining the effective amount of the therapeutic agent to be administered in the treatment of, or prophylaxis against, the condition(s), the physician may evaluate progression of the condition(s). In any event, suitable dosages of the therapeutic agents of the invention may be readily determined by those of skill in the art. Such dosages may be in the order of nanograms to milligrams of the therapeutic agents of the invention.

In order that the invention may be readily understood and put into practical effect, particular preferred embodiments will now be described by way of the following non-limiting examples.

### EXAMPLES

### Example 1

Expression of KLK5 and KLK7 in Normal Ovaries and Ovarian Tumors

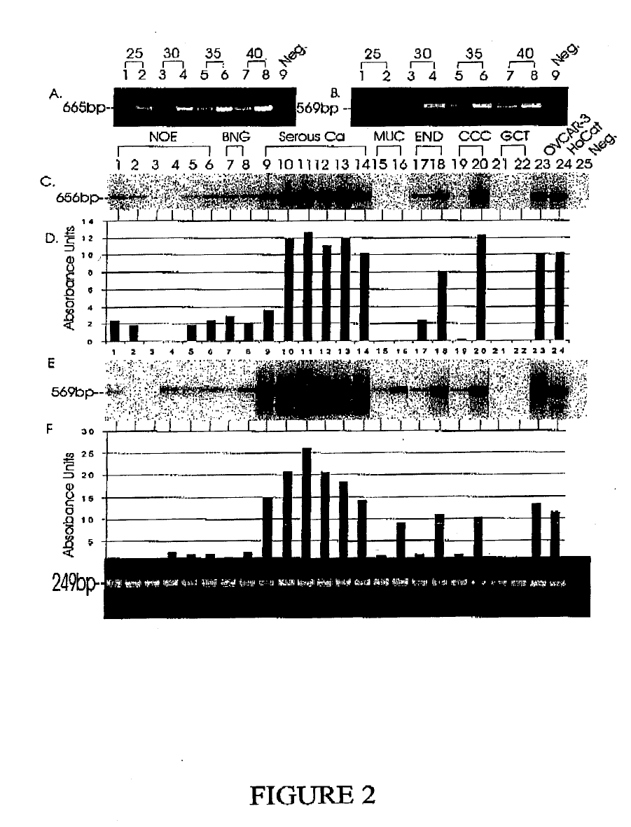

Six normal ovaries, thirty-one different ovarian tumors, and eight serous (SER) ovarian cancer cell lines were examined for their expression of KLK5 and KLK7 by RT-PCR over 35 cycles, followed by Southern blot and densitometry analyses. The results for KLK5 and KLK7 expression in a representative group of samples are shown in FIG. 2C-F respectively. β2-microglobulin, which was used as an internal control (FIG. 2G), showed a consistent pattern of expression in all samples indicating the integrity of the RNA. The KLK5 and KLK7 PCR products from normal ovarian epithelial (NOE) cells, the primary cultured cells from a serous ovarian carcinoma and the ovarian cancer cell line OVCAR-3 (Lane 6, 10 and 23 in FIG. 2C and 2E) were sequenced and were identical to that previously reported (23, 24). The KLK5 and KLK7 expression results and clinical information on all tumor tissues and cell lines are summarized in Table 1.

KLK5 expression was detected in the normal ovaries (4/6), as well as serous epithelial ovarian tumors (benign—2/2, malignant—11/11, cell lines 7/8), endometrioid carcinomas (4/5) and clear cell carcinomas (2/3). On densitometric analysis, higher level of KLK5 expression (>5 AU) was observed in 9 of 11 SER carcinomas and six of 8 SER-derived cancer cell lines compared with the normal ovaries, benign adenomas, END and clear cell carcinomas that are positive. However, no KLK5 was detected in mucinous tumors (0/4) or granulosa cell tumors (0/6) (FIG. 2C-D, Table 1). The expression pattern for KLK7 was similar to KLK5 (normal ovaries—4/6, benign adenoma—2/2, serous carcinomas—11/11, serous cancer cell lines—5/8, mucinous tumors—4/4, endometrioid carcinomas—5/5 and clear cell carcinomas—3/3) (FIG. 2E-F and Table 1). Ten of 11 SER carcinomas and five of 8 SER cancer cell lines showed higher KLK7 level (>5 AU) than normal ovaries, benign adenomas, and MUC, END, and clear cell carcinomas, while no KLK7 mRNA was found in granulosa cell tumors (FIG. 2E, Table 1). In addition, eight SER carcinomas with high KLK5 levels also showed high KLK7 expression and comparable levels of expression for both genes were observed in all samples. The present results suggest that both KLK5 and KLK7 are coordinately highly expressed by epithelial ovarian carcinomas, especially serous carcinomas. All of the patients with <3 year survival had high KLK5 and/or KLK7 levels (>5 AU), though the sample number is rather small (Table 1).

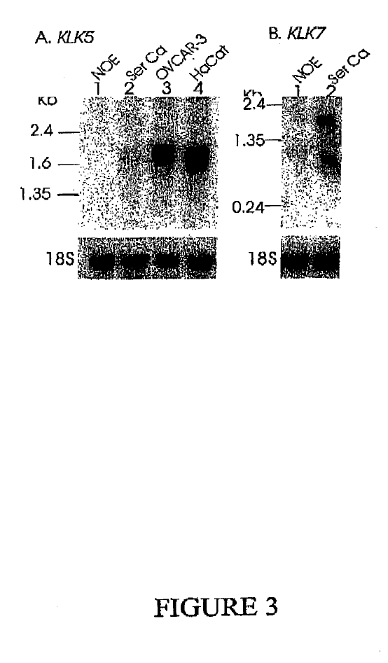

Northern blot hybridization with the full-length ³²P-labeled KLK5 cRNA probe showed a faint band in the serous ovarian carcinoma and an intense KLK5 transcript of 1.6-1.7 kb in the ovarian cancer cell line OVCAR-3 and HaCat control, whereas no visible band was detected in normal ovary (FIG. 3A). In addition, OVCAR-3 showed a larger KLK5 mRNA transcript than the human keratinocyte cell line (HaCat) (14). Northern blot hybridization with a ³²P-labeled KLK7 probe revealed 1.2 kb and 2 kb transcripts, as was reported previously in skin and ovarian cancer tissues respectively (16, 20) in the SER ovarian carcinoma sample but not the NOE cells. These results confirmed the findings of the RT-PCR and Southern blot analyses that both KLK5 and KLK7 are more highly expressed in serous carcinomas compared to normal tissues. Hybridization with ³²P-labeled 18S was used as an internal control and showed the relatively equivalent loading of RNA.

Methods

Tumor Samples and Cell Culture

The ovarian tissue samples, cell lines and their culture conditions used for this study were as described previously (5). Briefly, normal ovaries and ovarian tumor samples were obtained at surgery from women who underwent laparotomy. Ethics approval was obtained from the respective institutional Ethics Committees and informed consent was obtained from all patients. Epithelial cells from normal, benign and malignant ovaries were isolated from some of these tissue samples and the primary cultured cells were grown in M199 (Sigma, St. Louis, Mo., USA) and MCDB 105 (Sigma) media supplemented with 10% Fetal Calf Serum (FCS) and 10 ng/ml human epidermal growth factor (Boehringer Mannheim, Germany) (21). The ovarian cancer cell lines were grown in RPMI (Life Technologies, Inc., Gaithersburg, Md., USA) supplemented with 10% FCS. The conditioned media, supplemented with 0.05% bovine albumin instead of FCS, was collected after 48 hr cell culture.

Reverse Transcription-PCR (RT-PCR), Southern Blot and DNA Sequencing Analysis

Total RNA was isolated from tumor cells or tissues using TRIzol reagent (Life Technologies Inc.) following the manufacturer's instructions. Two μg of total RNA was reverse-transcribed into first-strand cDNA with random primers (100 ng) using Superscript II in a 25 μl reaction. PCR was performed with 50 ng primers (K5E×3S and K5E×6AS, FIG. 1A or K7E×3S and K7E×6AS, FIG. 1B) and 1 μl of cDNA from 2 different ovarian samples (normal ovarian epithelial cells and serous cancer cells), for 25, 30, 35 and 40 cycles to determine that amplification was in the linear range (FIGS. 2A and 2B). The final optimum cycling conditions chosen for further analysis were 94° C. for 5 min followed by 35 cycles of 94° C., 64° C. (KLK5) or 60° C. (KLK7) and 72° C. for 1 min each, and a final extension at 72° C. for 7 min. PCR for β2-microglobulin which was used as an internal control, was performed with the following primers—5′-TGAATTGCTATGTGTCTGGGT-3′ [SEQ ID NO: 23] and 5′-CCTCCATGATGCTGCTTACAT-3′, [SEQ ID NO: 24] for 35 cycles with similar PCR conditions except for the annealing temperature (56° C.).

To examine expression of KLK5 and KLK7 in normal ovaries and ovarian tumors, PCR was performed with the specific primers K5EX3S/K5EX6AS (FIG. 1A) and K7EX3S/K7EX6AS (FIG. 1B) with the conditions described as above. The PCR product was electrophoresed on a 1.5% agarose gel and visualized by ethidium bromide staining. The resulting amplicons were analyzed by Southern blot hybridization using digoxigenin (DIG) 3′ end labeled internal KLK5 (5′-AGTGCACTTGGGGGCTCTTGGTT-3′) [SEQ ID NO: 25] or KLK7 (5′-GCCGAGGTGCACGGTGTACTC-3′) [SEQ ID NO: 26] oligonucleotide probes, in Easyhyb® solution (Roche, Germany) overnight at 37° C. Washes with 0.2× sodium chloride/sodium citrate (SSC)/0.1% sodium dodecyl sulphate (SDS) were performed at 37° C. The membrane was blocked and then incubated with anti-DIG antibody, and signals were detected by CDP-star (Roche) using X-ray film. The intensity of the bands on the Southern blots was determined by densitometry (GS-690 Imaging, Bio-Rad) using the Bio-Rad Multi-Analyst™ program (Absorbance Units=AU). Representative PCR products (normal ovarian epithelial cells, NOE #5, serous ovarian carcinoma cells #11, and the ovarian cancer cell line OVCAR-3 from Table 2) were also purified (Qiagen, Pty Ltd, Australia), sequenced and then the DNA sequences were analyzed using tBLASTN on National Center for Biotechnology Information (NCBI).

Northern Blot Analysis

Ten μg of total RNA was denatured and electrophoresed on a 1% formaldehyde-agarose gel in 1× MOPS buffer, followed by capillary transfer in 20×SSC according to standard procedures. Prehybridisation was performed in UltraHyb (Clontech Laboratories, Inc., CA, USA) for 2 hr (68° C.). The constructs for the KLK5 and KLK7 cRNA probes were derived from the full length EST cDNA clone (W07551) (KLK5), and an exon 3-6 PCR product (KLK7, 569 bp) cloned into pGEMT (Promega, Madison, Wisc., USA). After linearising with XhoI, ³²P-labeled KLK5 and KLK7 cRNA probes were made using the Strip-EZT™ kit (Ambion, Inc., Austin, Tex., USA), and hybridization was performed overnight at 68° C. The membrane was then washed in 2×SSC with 0.1% SDS at 55° C. for 10 min, 0.1×SSC with 0.1% SDS at 60° C. for 20 min, followed by 0.1×SSC with 0.1% SDS at 70° C. for 20 min, and exposed to X-ray film at −80° C. for 24 hr for visualization. A control hybridization to determine RNA loading was performed with a ³²P-labeled 18S oligonucleotide probe (5′-CGGCATGTATTAGCTCTAGAATTACCACAG-3′) [SEQ ID NO: 22].

### Example 2

Identification of a Novel KLK5 5′UTR and its Splice Variant

Analysis of the EST database suggested that the differences in the KLK5 transcript length may be due to differences in the 5′UTR region. To determine the 5′UTR sequence of KLK5 in the normal ovary and ovarian cancer cell line OVCAR-3, RT-PCR and semi-nested PCR were performed with KLK5 specific primers (K5StartS, K5E×4AS and K5E×3AS, FIG. 1A). Two PCR products of 290 and 494 bp (FIG. 4A) were noted in OVCAR-3 cells but only the 494 bp band in NOE cells. Comparison of these two sequences to the published genomic sequence (GenBank No: AF135028) revealed two novel 5′UT sequences derived from a new exon further upstream of the known exon 1 (exon 2 in FIG. 4B). One of these novel 5′UT sequences (314 bp, GenBank Accession No: AF435980) was found in both NOE and OVCAR-3, while the shorter new exon sequence (112 bp, GenBank Accession No: AF435981) was found in OVCAR-3 only (FIG. 4B). The known 5′UT exon 1 derived sequence from HaCat cells (14) is identical to the last 31 bp at the 3′ end of the KLK5 long exon 1 sequence. The two novel KLK5 mRNA variants have an identical coding region as previously published (14, 23), and they would be translated into an identical protein, though their 5′UTR nucleotide sequences are different. Possibly this would also account for the small size difference between the OVCAR-3 and HaCat KLK5 transcripts seen on the Northern blot (FIG. 3A).

Methods

To determine the sequence of the different KLK5 mRNA transcripts between OVCAR-3 and HaCat cell lines as observed on Northern blot analysis (FIG. 3A); the known sequence of KLK5 (GenBank Accession No NM_{—}012427), originally derived from HaCat cells, was subjected to homology search using the tBLASTN algorithm on the NCBI web server against the human Expression Sequence Tag (EST) database. An EST from an ovarian adenocarcinoma clone (GenBank Accession No BF033594) revealed a different 5′UTR sequence; accordingly, primers were designed and RT-PCR was performed. Two μg of total RNA from OVCAR-3 and NOE cells was reverse-transcribed into first-strand cDNA with the KLK5 specific primer (K5E×4AS, FIG. 1A) using Superscript II in a 25 μl reaction. PCR was performed with 1 μl of cDNA, 50 ng of KLK5 specific primers (K5StartS, K5E×4AS, FIG. 1A), followed by a semi-nested PCR (K5StartS and K5E×3AS, FIG. 1A) with similar PCR conditions as described above.

### Example 3

Identification of KLK7 Splicing Variant mRNA Transcripts from Ovarian Cancer Analysis of the GenBank and EST database suggested that the differences in the length of the two KLK7 transcripts may be due to differences in the 5′UTR and/or 3′UTR regions. Using RT-PCR, 3′-RACE and sequencing analyses, two KLK7 mRNA transcripts were identified from NOE and PEO1. The long KLK7 transcript (1756 bp) (GenBank Accession No: AF411214, FIG. 4C) has 6 exons and contains a longer exon 1, which extends 144 nucleotides further upstream compared with the KLK7 gene sequence published in GenBank Accession No. AF332583, but the coding exons are consistent with this previously published sequence. The 3′ UTR of this long form of KLK7 (748 nts) is smaller than a splice variant of KLK7 (1039 nts; SEQ IN NO: 15) disclosed in GenBank Accession No. 332583 but longer than another KLK7 splice variant (190 nts; SEQ IN NO: 13) disclosed in the same reference. The ATG codon is located at nucleotide (nt) 247 and a protein, hK7, of 253 amino acids (aa) is predicted (FIG. 4E), that is identical to the enzyme purified from human skin (16). In comparison to the long form, the short KLK7 mRNA transcript (1054 bp, GenBank Accession No: AF411215, FIG. 4D) contains 5 exons with exon 2 deleted and contains only 177 bp in the 3′UTR region. The short KLK7 transcript generates a protein of 181 aa as a different protein translation start site (ATG at nt 332) is utilized, and encodes a shortened protein sequence without a pre and pro region (FIG. 4E). Both the long and short KLK7 transcripts may account for the 2 kb and 1.2 kb on the Northern blots when allowing 100-200 bp for a poly A tail. The truncated hK7 does not contain the histidine residue of the catalytic triad (FIG. 4E). Both short and long KLK7 transcripts were expressed by NOE and PEO1 cells as determined by 3′-RACE and RT-PCR respectively although PEO1 cells predominantly expressed the longer transcript (FIG. 4F-H).

Method

To determine the precise sequences of the two different mRNA transcripts (1.2 kb and 2 kb respectively) found on Northern blot analysis (FIG. 3B), the published KLK7 sequence (GenBank Accession No NM_{—}005046), originally derived from human keratinocytes, was subjected to homology search using the tBLASTN algorithm on the NCBI web server against the EST database. An ovarian tumor EST clone (GenBank Accession No AU134435) showed further upstream 5′UTR sequence, while another one had further downstream 3′UTR sequence (GenBank Accession No AA425991). Accordingly four sets of primers, spanning these different regions, (FIG. 1B) were designed and RT-PCR was performed with these primers and cDNA from the ovarian cancer cell line PEO1 with NOE as the control. After KLK7 gene specific RT with K7E×3AS primer, PCR was performed with 1 μl of cDNA and 50 ng of primers (K7StartS and K7E×4AS, FIG. 1B) with the conditions 94° C. for 5 min followed by 35 cycles of 94° C., 58° C. and 72° C. for 1 min each, and a final extension at 72° C. for 7 min. To examine the 3′UTR sequence of KLK7, 3′-rapid amplification of cDNA end (3′-RACE) was firstly performed with the universal 3′UTR primer (Promega, Madison, Wisc., USA), and PCR amplification was then performed with 1 μl of cDNA, 50 ng of KLK7 specific primers (K75′UTR and K73′UTshAS, FIG. 1B) with the similar PCR conditions as described above. To determine the long KLK7 mRNA transcripts, PCR was performed with K7E×2S and K7 3′UTAS (FIG. 1B).

### Example 4

Western Blot Analysis of hK5 and hK7

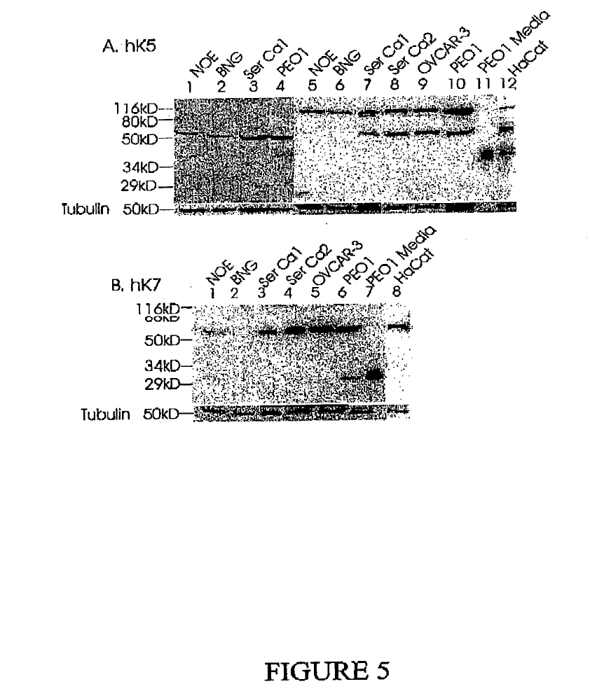

Eight different ovarian cancer cell lines, primary cultured benign and malignant ovarian epithelial cells were analyzed by Western blot with anti-hK5 and anti-hK7 antibodies respectively (FIG. 5). FIGS. 5A and B show a representative Western blot with anti-hK5 antibodies of the primary cultured NOE cells, BNG adenoma cells, serous ovarian carcinoma cells and ovarian cancer cell lines. The anti-hK5 pro-region antibody detected a 60 kDa protein in these cells (FIG. 5A Lanes 1-4), and a faint band (≈40 kDa) was seen in PEO1 cells. The anti-hK5 active region peptide antibody detected 100 kDa, 60 kDa and 36 kDa bands from HaCat cells as previously described with the pro-region antiserum (17). The 100 kDa and 60 kDa bands were seen in the extracts of ovarian cancer cells. However, a 36 kDa band was detected in the conditioned media of PEO1 cells by the anti-hK5 active region antibody (FIG. 5A), but not detected by the pro-region antibody (data not shown). The anti-hK7 antibody recognized proteins of 60 kDa and 30 kDa in the HaCat control and ovarian cancer cells (FIG. 5B) but only a 30 kDa band from the conditioned media of PEO1. Primary cultured ovarian cancer cells and cell lines showed higher hK5 and hK7 expression than NOE and adenoma cells (FIGS. 5A and B). In addition, hK5 and hK7 were detected from the conditioned media of ovarian cancer cell line PEO1 suggesting that these enzymes can be secreted by cancer cells. Western blot analysis of β-tubulin expression showed the relatively equal loading of protein.

Method

Cytoplasmic extracts (150 μg protein) from primary cultured NOE cells, ovarian tumor cells (BNG, Ser Ca 1 and 2, OVCAR-3 and PEO1), HaCat cells and conditioned media from ovarian cancer cell line PEO1 were electrophoresed on 10% SDS-polyacrylamide gels, and the protein was then transferred to a Protran membrane (Schleicher and Schuell, Dassel, Germany). After confirming equivalent protein loading by Ponceau S (Sigma) staining, the membrane was blocked with 5% skim milk in TBS/Tween-20 2 hours at room temperature and then incubated with anti-hK5-proregion antibody (Pe-Cl, 0.4 μg/ml), anti-hK5 active region peptide antibody (0.2 μg/ml) and anti-hK7 antibody (anti-SCCE antibody, 0.6 μg/ml) overnight at 4° C., respectively. The production, specificity and characterization of Pe-C1 and anti-hK7 antibodies have been previously described (17, 22). Anti-hK5 active region antibody is a polyclonal rabbit antiserum raised against and affinity purified towards a mixture of the peptides—RIRPTKDVRPINVSSHC [SEQ ID NO: 27] (mid region) and CEDAYPRQIDDTMF [SEQ ID NO: 28] (C-terminal). The blot was washed and then incubated (1 hr) with a horseradish peroxidase goat anti-rabbit IgG (Dako, Glostrup, Denmark) ( 1/2,000 dilution) at room temperature. The signals were visualized on X-ray film by enhanced chemiluminescence. After stripping the original membrane, Western blot analysis was performed with β-tubulin antibody ( 1/2000 dilution, PIERCE, Rockford, Ill. USA) as an internal control for equal loading.

### Example 5

Expression of hK5 and hK7 in Ovarian Cancer Tissues

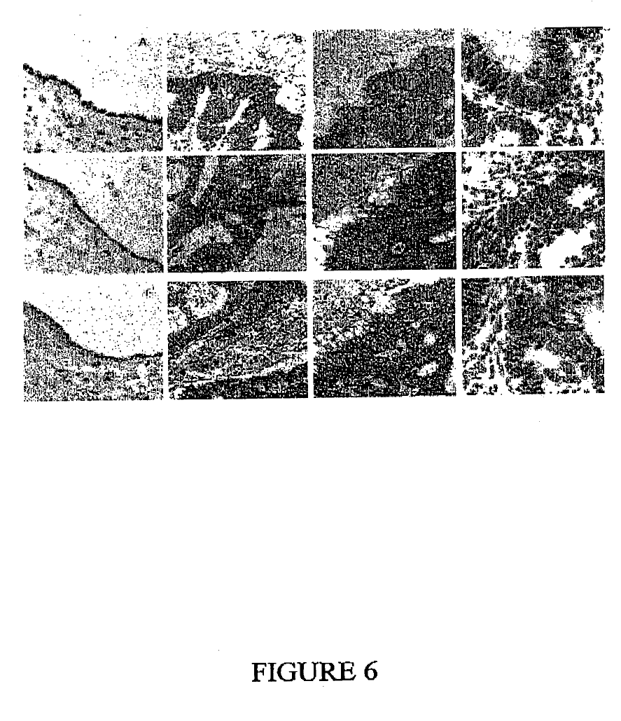

On immunohistochemistry, using either anti-hK5 pro-region or active form region antibody, only weak hK5 staining was observed in the surface epithelium of benign serous cystadenoma cells (FIGS. 6A and 6E), while hK5 was found predominantly at the apical membrane and cytoplasm of cancer cells (FIGS. 6B-C and 6F-G). In addition, the staining intensity detected by the anti-active form peptide antibody was stronger than that of the anti-pro-form antibody. It also appears that extracellular secretion of hK5 was detected as immunoreactive material was detected at the apical membrane of these glandular structures (FIG. 6G), which is consistent with our finding on Western blot analysis (FIG. 5A). Using the anti-hK7 antibody, only weakly focal hK7 staining was found in benign serous cystadenoma cells (FIG. 6I), while strong staining was observed in the apical membrane and cytoplasm of ovarian carcinoma cells (FIG. 6J-K), and the cancer cells at the invasive front (FIG. 6J). Like hK5, extracellular secretion of hK7 from the cancer cells was also observed (FIG. 6K). No staining was observed in the negative control where 10% normal goat serum replaced the primary antibodies (FIGS. 6D, H and L).

Method

Formalin fixed paraffin blocks from 4 serous ovarian tumors and 2 normal ovaries were sectioned (4 μm), deparaffinized and rehydrated, and then antigen retrieval was performed by microwave heat treatment in 5% urea in 0.1M Tris buffer (pH 9.5). Following H₂O₂ treatment and blocking endogenous peroxidase, the sections were incubated overnight with anti-hK5-proregion antibody (3 μg/ml), anti-hK5 active region peptide antibody (1 μg /ml) or anti-hK7 antibody (3 μg/ml) at 4° C. respectively. Then the EnVision⁺™ peroxidase (anti-rabbit) polymer (Dako, Calif., USA) was used following the manufacturer's instructions. Peroxidase activity was detected using 3,3′-diaminobenzidine (DAB) (Sigma) as the chromogen with H₂O₂ as the substrate. The sections were counterstained with Mayer hematoxylin. Normal rabbit serum replaced primary antibodies was used as negative controls.

Discussion of the Examples

In this study, the majority of epithelial carcinomas analyzed are from late stage (III or IV) disease. Moreover, hK6 (9), hK10 (26) and hK11 (27) have been found to be higher in the serum of ovarian cancer patients, and their potential as tumor markers for the diagnosis of this cancer has been suggested. The results presented herein show that hK5 and hK7 can be secreted by ovarian cancer cells, suggesting they have similar potential as serum markers for the diagnosis and monitoring of this disease.

Previous studies suggested that hK7 can degrade the cohesive structures between individual comeocytes in the stratum corneum that is the genesis for cellular desquamation or shedding of skin cells (16, 28). The data from the present study, that the invading cancer cells expressed high hK7, suggests that hK7 may have the potential to degrade the surrounding matrix as the tumor progresses. The present inventors also showed that those serous carcinoma cells with high KLK5 levels had concordantly high KLK7 levels, and the identical localization of hK5 and hK7 in ovarian cancer tissues. In previous studies, others have shown the co-expression of hK5 and hK7 in skin tissue and suggested that there may be a relationship between these two enzymes (14, 17). Indeed, they have now confirmed the interaction of hK5 and hK7 in vitro in an activation cascade (⁴Brattsand et al., unpublished). Thus, it is reasonable to hypothesize a similar role of these two enzymes in ovarian tumor invasion.

This process of tumor invasion is a complex event involving the activation and inhibition of these serine proteases during tumor progression. The secreted form, seen on Western blot analysis, is at the theoretical molecular weight for these enzymes (36 kDa for hK5 and 30 kDa for hK7), but the high molecular weight protein bands of hK5 or hK7 in ovarian cancer samples are likely complexes of these enzymes possibly with their different respective inhibitors (17). That the antibody to the hK5 pro-form only detects the 60 kDa band suggests that this is either the pro-form only or a complex of the pro-form with another protein or perhaps a dimer of the pro-form. The substantial apparent molecular weight difference of 26 kDa between the active form (36 kDa) and the pro-form (60 kDa) is unlikely to be entirely due to the clipping of the pro-region only (66 aa), so that the latter 2 scenarios are more likely. Similarly, the 60 kDa bK7 band likely represents a dimer or inhibitor complex. In addition, the NOE and BNG cells showed weak hK5 immuno-activity as detected by the anti-pro-region antibody, or only higher molecular weight bands (100 kDa) detected by the anti-active form antibody. These results suggest that all of the hK5 is bound in an inhibitor complex and that no active hK5 was present in these cells. The presence of such inhibitor(s) might modulate the shedding of tumor cells so that identification of these inhibitors.

In addition to the high expression of KLK5 /hK5 and KLK7 AIK7 in ovarian carcinomas, two novel KLK5 splice variants with an additional upstream exon were found in the ovarian cancer cell line OVCAR-3, while only one transcript was found in NOE cells. We also identified two KLK7 mRNA transcripts from NOE and PEO1 cells. These two KLK7 transcripts have a similar exon 1 (which is larger than previously published), while the short form, which is predominantly expressed by NOE cells, has no exon 2, and has only 177 bp in 3′UTR. The KLK5 mRNA variants with different 5′UTR can be translated into the same hK5 protein. However, the 2 KLK7 transcripts encode different proteins. Multiple mRNA variants with different 5′UTR sequences in several genes, such as mouse estrogen receptor a gene (29), have been reported in different tissues, and it is suggested that different promoter utilization and alternative splicing generate these mRNA variants often in a tissue-specific manner. The results presented herein show different 5′UTR sequences of KLK5 and KLK7 in ovarian tissues from HaCat cells, and suggest that different promoters of these 2 KLKs are present in these tissues. In addition, the present results show that there was selective use of the different KLK5 5′UTR sequences in ovarian cancer cells compared with NOE cells. Thus, the use of these variant KLK5 promoters may be important in tumorigenesis, as their normal regulation may be altered in cancer cells.

In summary, the results presented herein demonstrate the differential expression of KLK5/hK5 and KLK7/hK7 between normal ovaries and ovarian carcinomas, particularly high expression in serous carcinomas. The presence of different mRNA transcripts of KLK5 and KLK7 in normal ovarian epithelial cells compared with ovarian cancer suggests that these variant KLK transcripts are important in ovarian tumorigenesis and their potential as biomarkers for cancer. The secretion of hK5 and hK7 by ovarian cancers may allow their use as potential serum markers for this tumor, possibly in a multiplex kallikrein profile. The concordant high expression of both KLK5 and KLK7 in ovarian cancer further supports the hypothesis that these two enzymes are involved in an activation cascade.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### BIBLIOGRAPHY

1. Risch, H. A. Hormonal etiology of epithelial ovarian cancer, with a hypothesis concerning the role of androgens and progesterone, J. Natl. Cancer. Inst., 90: 1774-1786, 1998.
2. Landis, S. H., Murray, T., Bolden, S., and Wingo, P. A. Cancer statistics, 1998.
3. Harvey, T. J., Hooper, J. D., Myers, S. A., Stephenson, S. A., Ashworth, L. K., and Clements, J. A. Tissue-specific expression patterns and fine mapping of the human kallikrein (KLK) locus on proximal 19q13.4, J. Biol. Chem., 275: 37397-37406, 2000.
4. Yousef, G. M., Chang, A., Scorilas, A., and Diamandis, E. P. Genomic organization of the human kallikrein gene family on chromosome 19q13.3-q13.4, Biochem. Biophys. Res. Commun., 276: 125-133, 2000.
5. Dong, Y., Kaushal, A., Bui, L., Chu, S., Fuller, P. J., Nicklin, J., Samaratunga, H., and Clements, J. A. Human kallikrein 4 (klk4) is highly expressed in serous ovarian carcinomas, Clin. Cancer Res., 7: 2363-2371, 2001.
6. Obiezu, C. V., Scorilas, A., Katsaros, D., Massobrio, M., Yousef, G. M., Fracchioli, S., Rigault De La Longrais, I. A., Arisio, R., and Diamandis, E. P. Higher human kallikrein gene 4 (klk4) expression indicates poor prognosis of ovarian cancer patients, Clin. Cancer Res., 7: 2380-2386, 2001.
7. Anisowicz, A., Sotiropoulou, G., Stenman, G., Mok, S. C., and Sager, R. A novel protease homolog differentially expressed in breast and ovarian cancer, Mol. Med., 2: 624-636, 1996.
8. Tanimoto, H., Underwood, L. J., Shigemasa, K., Parmley, T. H., and O'Brien, T. J. Increased expression of protease M in ovarian tumors, Tumour Biol., 22: 11-18, 2001.
9. Diamandis, E. P., Yousef, G. M., Soosaipillai, A. R., and Bunting, P. Human kallikrein 6 (zyme/protease M/neurosin): a new serum biomarker of ovarian carcinoma, Clin. Biochem., 33: 579-583, 2000.
10. Underwood, L. J., Tanimoto, H., Wang, Y., Shigemasa, K., Parmley, T. H., and O'Brien, T. J. Cloning of tumor-associated differentially expressed gene-14, a novel serine protease overexpressed by ovarian carcinoma, Cancer Res., 59: 4435-4439, 1999.
11. Magklara, A., Scorilas, A., Katsaros, D., Massobrio, M., Yousef, G. M., Fracchioli, S., Danese, S., and Diamandis, E. P. The human KLK8 (neuropsin/ovasin) gene: identification of two novel splice variants and its prognostic value in ovarian cancer, Clin. Cancer Res., 7: 806-811, 2001.
12. Yousef, G. M., Kyriakopoulou, L. G., Scorilas, A., Fracchioli, S., Ghiringhello, B., Zarghooni, M., Chang, A., Diamandis, M., Giardina, G., Hartwick, W. J., Richiardi, G., Massobrio, M., Diamandis, E. P., and Katsaros, D. Quantitative Expression of the Human Kallikrein Gene 9 (KLK9) in Ovarian Cancer: A New Independent and Favorable Prognostic Marker, Cancer Res., 61: 7811-7818, 2001.
13. Luo, L. Y., Bunting, P., Scorilas, A., and Diamandis, E. P. Human kallikrein 10: a novel tumor marker for ovarian carcinoma? Clin. Chim. Acta., 306: 111-118, 2001.
14. Brattsand, M. and Egelrud, T. Purification, molecular cloning, and expression of a human stratum corneum trypsin-like serine protease with possible function in desquamation, J. Biol. Chem., 274: 30033-30040, 1999.
15. Egelrud, T. Purification and preliminary characterization of stratum comeum chymotryptic enzyme: a proteinase that may be involved in desquamation, J. Invest. Dermatol., 101: 200-204, 1993.
16. Hansson, L., Stromqvist, M., Backman, A., Wallbrandt, P., Carlstein, A., and Egelrud, T. Cloning, expression, and characterization of stratum corneum chymotryptic enzyme. A skin-specific human serine proteinase, J. Biol. Chem., 269: 19420-19426, 1994.
17. Ekholm, T. E., Brattsand, M., and Egelrud, T. Stratum corneum tryptic enzyme in normal epidermis: a missing link in the desquamation process? J. Invest. Dermatol., 114: 56-63, 2000.
18. Gan, L., Lee, I., Smith, R., Argonza-Barrett, R., Lei, H., McCuaig, J., Moss, P., Paeper, B., and Wang, K. Sequencing and expression analysis of the serine protease gene cluster located in chromosome 19q13 region, Gene, 257: 119-130, 2000.
19. Kim, H., Scorilas, A., Katsaros, D., Yousef, G. M., Massobrio, M., Fracchioli, S., Piccinno, R., Gordini, G., and Diamandis, E. P. Human kallikrein gene 5 (KLK5 ) expression is an indicator of poor prognosis in ovarian cancer, Br. J. Cancer, 84: 643-650, 2001.
20. Tanimoto, H., Underwood, L. J., Shigemasa, K., Yan Yan, M. S., Clarke, J., Parmley, T. H., and O'Brien, T. J. The stratum corneum chymotryptic enzyme that mediates shedding and desquamation of skin cells is highly overexpressed in ovarian tumor cells, Cancer, 86: 2074-2082, 1999.
21. Godwin, A. K., Testa, J. R., Handel, L. M., Liu, Z., Vanderveer, L. A., Tracey, P. A., and Hamilton, T. C. Spontaneous transformation of rat ovarian surface epithelial cells: association with cytogenetic changes and implications of repeated ovulation in the etiology of ovarian cancer, J. Natl. Cancer Inst., 84: 592-601, 1992.
22. Sondell, B., Dyberg, P., Anneroth, G. K., Ostman, P. O., and Egelrud, T. Association between expression of stratum corneum chymotryptic enzyme and pathological keratinization in human oral mucosa, Acta. Derm. Venereol., 76: 177-181, 1996.
23. Yousef, G. M. and Diamandis, E. P. The new kallikrein-like gene, KLK-L2. Molecular characterization, mapping, tissue expression, and hormonal regulation, J. Biol. Chem., 274: 37511-37516, 1999.
24. Yousef, G. M., Scorilas, A., Magklara, A., Soosaipillai, A., and Diamandis, E. P. The KLK7 (PRSS6) gene, encoding for the stratum comeum chymotryptic enzyme is a new member of the human kallikrein gene family—genomic characterization, mapping, tissue expression and hormonal regulation, Gene, 254: 119-128, 2000.
25. Noel, A., Albert, V., Bajou, K., Bisson, C., Devy, L., Frankenne, F., Maquoi, E., Masson, V., Sounni, N. E., and Foidart, J. M. New functions of stromal proteases and their inhibitors in tumor progression, Surg. Oncol. Clin. N. Am., 10: 417-32, x-xi, 2001.
26. Luo, I., Bunting, P., Scorilas, A., and Diamandis, E. P. Human kallikrein 10: a novel tumor marker for ovarian carcinoma? Clinica. Chimica. Acta., 306: 110-118, 2001.
27. Diamandis, E. P., Okui, A., Mitsui, S., Luo, L. Y., Soosaipillai, A., Grass, L., Nakamura, T., Howarth, D. J., and Yamaguchi, N. Human Kallikrein 11: A New Biomarker of Prostate and Ovarian Carcinoma, Cancer Res., 62: 295-300, 2002.
28. Egelrud, T., Regnier, M., Sondell, B., Shroot, B., and Schmidt, R. Expression of stratum corneum chymotryptic enzyme in reconstructed human epidermis and its suppression by retinoic acid, Acta. Derm. Venereol., 73: 181-184, 1993.
29. Kos, M., O'Brien, S., Flouriot, G., and Gannon, F. Tissue-specific expression of multiple mRNA variants of the mouse estrogen receptor alpha gene, FEBS Lett., 477: 15-20, 2000.

30. Serov, S. F., Scully, R. E. Histological typing of ovarian tumour. World Health Organisation, 9, 1973.

TABLE 1
Patient Characteristics and Expression Patterns of KLK5 and KLK7.

| | | | | Intensity^{c} and | Intensity and |
|---|---|---|---|---|---|
| | | | Survival in | (Summary) of KLK5 | (Summary) of KLK7 |
| Number | Histology | Stage^{a}/Grade^{b} | Months | expression | expression |
| 1 | NOT^{d} | | | 2.3 | 1.2 |
| 2 | NOT | | | 1.8 | 0 |
| 3 | NOT | | | 0 | 0 |
| 4 | NOE | | | 0 | 2.4 |
| 5 | NOE | | | 1.8 | 1.8 |
| 6 | NOE | | | 2.3 (4/6) | 2 (4/6) |
| 7 | SER³adenoma | | | 2.9 | 1.3 |
| 8 | SER adenoma | | | 2 (2/2) | 2.4 (2/2) |
| 9 | SER Ca | IIb/2 | 20D^{e} | 3.6 | 14.8 |
| 10 | SER Ca | IIc/3 | 2D | 11.8 | 20.6 |
| 11 | SER Ca | IIIc/1 | 66 | 12.6 | 25.9 |
| 12 | SER Ca | IV/2-3 | 22D | 11 | 20.6 |
| 13 | SER Ca | IIIc/3 | 25D | 11.9 | 18.4 |
| 14 | SER Ca | IIIc/3 | 19D | 10.1 | 14.2 |
| 15 | SER Ca | IIIb/3 | 18D | 6.6 | 18.4 |
| 16 | SER Ca | III/1 | 162 | 1 | 10 |
| 17 | SER Ca Tissue | III/3 | 14 | 10 | 12 |
| 18 | SER Ca Tissue | III/2-3 | 16 | 9 | 11 |
| 19 | SER Ca Tissue | III/3 | 19 | 9 (11/11) | 4 (11/11) |
| 20 | JAM (SER) | Xenograft/3 | | 1 | 0 |
| 21 | CI-80-13S (SER) | IV/3 | | 12.1 | 5 |
| 22 | SKOV-3 (SER) | III/1 | | 0 | 0 |
| 23 | OVCAR-3 (SER) | III/NA | | 10.1 | 12.1 |
| 24 | PEO1 (SER) | III/3 | | 11 | 10.3 |
| 25 | PEO4 (SER) | Recurrent | | 10 | 12 |
| 26 | PEO14 (SER) | III/1 | | 9.9 | 10 |
| 27 | OAW42 (SER) | III/NA | | 12 (7/8) | 0 (5/8) |
| 28 | MUC adenoma | | | 0 | 5.1 |
| 29 | MUC Ca | I/NA | NA | 0 | 8.9 |
| 30 | MUC Ca | I/NA | NA | 0 | 2.5 |
| 31 | MUC Ca | II/NA | NA | 0 (0/4) | 1.6 (4/4) |
| 32 | END Ca | IIb/2 | 84 | 2.4 | 1.8 |
| 33 | END Ca | III/2-3 | 10D | 12.1 | 10.8 |
| 34 | END Ca | Ic/LMP | 48 | 8.1 | 24.5 |
| 35 | END Ca | Ia/NA | 69 | 0 | 6.6 |
| 36 | END Ca | IIIb/3 | 84 | 1 (4/5) | 7.6 (5/5) |
| 37 | CCC | Ia/2 | 124 | 0 | 1.8 |
| 38 | CCC | IIc/NA | 74 | 12.2 | 10.2 |
| 39 | CCC | IIIb/2 | 14D | 8 (2/3) | 18.6 (3/3) |
| 40 | GCT | I/NA | NA | 0 | 0 |
| 41 | GCT | I/NA | NA | 0 | 0 |
| 42 | GCT | I/NA | NA | 0 | 0 |
| 43 | GCT | Ia/NA | NA | 0 | 0 |
| 44 | GCT | Unstaged/NA | NA | 0 | 0 |
| 45 | GCT | Recurrent/NA | NA | 0 | 0 |

^{a}Federation of International Gynecology and Obstetrics (FIGO) stage system.
^{b}Grades: 1, well; 2, moderately; 3, poorly and 4, undifferentiated (30).
^{c}Intensity of KLK5 or KLK7 Southern blot bands determined by densitometric analysis (absorbance units).
^{d}NOT, normal ovarian tissues; NOE, normal ovarian epithelial cells; SER, serous; MUC, mucinous; END, endometrioid; CCC, clear cell carcinoma; GCT, granulosa cell tumor; and NA, not available.
^{e}D, died from this cancer; uninitialed numbers indicate still alive or died from other causes.

What is claimed is:

1. A method for detecting the presence or diagnosing the risk of a cancer or related condition in a patient, comprising detecting the presence of an aberrant KLK5 expression product or the aberrant expression of a KLK7 expression product in a biological sample obtained from the patient, wherein the aberrant expression product or the aberrant expression correlates with the presence or risk of the cancer or related condition.

2. The method of claim 1, wherein the cancer is regulatable by a hormone including, but not restricted to, testosterone, estrogen and progesterone.

3. The method of claim 1, wherein the cancer is a serous cancer,.

4. The method of claim 3, wherein the serous cancer is a serous carcinoma.

5. The method of claim 3, wherein the serous cancer is ovarian cancer.

6. The method of claim 1, wherein the aberrant KLK5 expression product is an aberrant KLK5 polynucleotide, which comprises a deletion of one or more nucleotides relative to normal KLK5.

7. The method of claim 6, wherein the aberrant KLK5 polynucleotide comprises a deletion corresponding to all or part of exon 1 of normal KLK5.

8. The method of claim 6, wherein the aberrant KLK5 polynucleotide comprises a deletion comprising all or part of the sequence set forth in SEQ ID NO: 5.

9. The method of claim 6, wherein the aberrant KLK5 polynucleotide comprises the sequence set forth in SEQ ID NO: 9.

10. The method of claim 1, wherein the aberrant expression of the KLK7 expression product is represented by a level or functional activity of the KLK7 expression product, which differs from a normal reference level and/or functional activity of that expression product.

11. The method of claim 10, wherein the KLK7 expression product is expressed at a higher level or functional activity than the normal reference level and/or functional activity.

12. The method of claim 10, wherein the KLK7 expression product is a KLK7 polynucleotide comprising all or part of KLK7 exon 2.

13. The method of claim 10, wherein the KLK7 expression product is a KLK7 polynucleotide comprising the sequence set forth in SEQ ID NO: 16.

14. The method of claim 10, wherein the KLK7 expression product is a K7 polypeptide comprising all or part of the amino acid sequence set forth in SEQ ID NO: 21.

15. The method of claim 10, wherein the KLK7 expression product is an aberrant K7 polypeptide comprising the sequence set forth in SEQ ID NO: 17.

16. An isolated polynucleotide comprising a nucleotide sequence which corresponds or is complementary to at least a portion of an aberrant KLK5 polynucleotide that correlates with the presence or risk of a cancer or related condition.

17. The polynucleotide of claim 16, wherein the aberrant KLK5 polynucleotide comprises a deletion corresponding to all or part of exon 1 of normal KLK5.

18. The polynucleotide of claim 16, wherein the aberrant KLK5 polynucleotide comprises a deletion comprising all or part of the sequence set forth in SEQ ID NO: 5.

19. The polynucleotide of claim 16, wherein the aberrant KLK5 polynucleotide comprises the sequence set forth in SEQ ID NO: 9.

20. A probe for interrogating nucleic acid for the presence of nucleic acid molecules that are associated with a cancer or a related condition, comprising a nucleotide sequence which corresponds or is complementary to a portion of an aberrant KLK5 polynucleotide.

21. An isolated polynucleotide comprising a nucleotide sequence which corresponds or is complementary to at least a portion of a KLK7 polynucleotide whose aberrant expression correlates with the presence or risk of a cancer or related condition, wherein the KLK7 polynucleotide comprises the sequence set forth in SEQ ID NO: 16.

22. A probe for interrogating nucleic acid for the presence of nucleic acid molecules that are associated with a cancer or a related condition, comprising a nucleotide sequence which corresponds or is complementary to a portion of a KLK7 polynucleotide whose aberrant expression correlates with the presence or risk of the cancer or related condition, wherein the KLK7 polynucleotide comprises the sequence set forth in SEQ ID NO: 16.

23. A vector comprising the polynucleotide of claim 16.

24. The vector of claim 23, wherein the polynucleotide is operably linked to a regulatory polynucleotide.

25. A vector comprising the polynucleotide of claim 22.

26. The vector of claim 28, wherein the polynucleotide is operably linked to a regulatory polynucleotide.

27. A cell line comprising at least a portion of an aberrant KLK5 polynucleotide that correlates with the presence or risk of a cancer or related condition.

28. A cell line comprising at least a portion of a KLK7 polynucleotide whose aberrant expression correlates with the presence or risk of a cancer or related condition, wherein the KLK7 polynucleotide comprises the sequence set forth in SEQ ID NO: 16.
